SEQUENCE LISTING (21 sequences)

SEQ ID NO: 1 - DNA, 17 bp, Human
cgtgcacgtcacgctag

SEQ ID NO: 2 - DNA, 21 bp, Human
gcacgtgcagtgcgatcttaa

SEQ ID NO: 3 - DNA, 23 bp, Human
gtgcgacgacatggactgcctgt

SEQ ID NO: 4 - DNA, 24 bp, Human
ggctgacaggttgatgttctgaag

SEQ ID NO: 5 - DNA, 21 bp, Human
caatccccatgacccccaatg

SEQ ID NO: 6 - DNA, 21 bp, Human
gatgggcttgagggaggagat

SEQ ID NO: 7 - DNA, 21 bp, Human
gtgggcgagatgacattgaag

SEQ ID NO: 8 - DNA, 20 bp, Human
cccgtggagacctggaagag

SEQ ID NO: 9 - DNA, 21 bp, Human
tttgtgctggaggctgtgctg

SEQ ID NO: 10 - DNA, 21 bp, Human
acctctgtgtgcccgtgggta

SEQ ID NO: 11 - DNA, 18 bp, Human
cgccctcccacctccatt

SEQ ID NO: 12 - DNA, 226 bp, Human
gtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcagggatggtcgagtactccctggaccttcagaacatcaacctgtcagcc

SEQ ID NO: 13 - DNA, 3693 bp, Human
gcagagccgaggcaggcgagagaccggcctcagccccgcgctccccgccgcgctggtctctcccgggcacttgccccgctgctcggtccccgcagcacccgcgcccccgccccggcccggagcgccgcgctcccgcccgccgcccgcccagagcctcgccgtccctccagcccgccgcctcctcctcctcccgccatggcccgggcccccgggcccgccgccccccgcccgccgccccccgcgccccgggggctcagctgatcctgaattggcggggggtcggctggcggggctgggggtccctctcgatcgccggcttcgggtggggacgcgttgtccccgcgtctctaacctggacgaccccccgcggccgggccacgtccatgccaagggctcccagctcagtgtggacatggctgagagcgcctccccgccctcctcatctgcagcagccccagccgctgagccaggagtcaccacggagcagcccggaccccggagccccccatcctccccgccaggcctggaggagcctctggatggagctgatcctcatgtcccacacccagacctggcgcctattgccttcttctgcctgcgacagaccaccagcccccggaactggtgcatcaagatggtgtgcaacccgtggtttgaatgtgtcagcatgctggtgatcctgctgaactgcgtgacacttggcatgtaccagccgtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcagggatggtcgagtactccctggaccttcagaacatcaacctgtcagccatccgcaccgtgcgcgtcctgaggcccctcaaagccatcaaccgcgtgcccaacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcaactttgacaacatcggttatgcttggattgtcatcttccaggtgatcactctggaaggctgggtggagatcatgtactacgtgatggatgctcactccttctacaacttcatctacttcatcctgcttatcatagtgggctccttcttcatgatcaacctgtgcctcgttgtcatagcgacccagttctcggagaccaagcaacgggagcaccggctgatgctggagcagcggcagcgctacctgtcctccagcacggtggccagctacgccgagcctggcgactgctacgaggagatcttccagtatgtctgccacatcctgcgcaaggccaagcgccgcgccctgggcctctaccaggccctgcagagccggcgccaggccctgggcccggaggccccggcccccgccaaacctgggccccacgccaaggagccccggcactaccagctgtgcccgcaacatagccccctggatgcgacgccccacaccctggtgcagcccatccccgccacgctggcttccgatcccgccagctgcccttgctgccagcatgaggacggccggcggccctcgggcctgggcagcaccgactcgggccaggagggctcgggctccgggagctccgctggtggcgaggacgaggcggatggggacggggcccggagcagcgaggacggagcctcctcagaactggggaaggaggaggaggaggaggagcaggcggatggggcggtctggctgtgcggggatgtgtggcgggagacgcgagccaagctgcgcggcatcgtggacagcaagtacttcaaccggggcatcatgatggccatcctggtcaacaccgtcagcatgggcatcgagcaccacgagcagccggaggagctgaccaacatcctggagatctgcaatgtggtcttcaccagcatgtttgccctggagatgatcctgaagctggctgcatttgggctcttcgactacctgcgtaacccctacaacatcttcgacagcatcattgtcatcatcaggcctcctactgctgcctcctacctgtaccctgggcctgccctgcgggaccgcagcatctgggagatcgtggggcaggcggacggtgggctgtcggtgctgcggaccttccggctgctgcgcgtgctgaaactggtgcgcttcatgcctgccctgcggcgccagctcgtggtgctcatgaagaccatggacaacgtggccaccttctgcatgctgctcatgctcttcatcttcatcttcagcatccttgggatgcatatttttggctgcaagttcagcctccgcacggacactggagacacggtgcccgacaggaagaacttcgactccctgctgtgggccatcgtcactgtgttccagatcctcacccaggaggactggaacgtcgttctctacaatggcatggcctccacttctccctgggcctccctctactttgtcgccctcatgaccttcggcaactatgtgctcttcaacctgctggtggccatcctggtggagggcttccaggcggagggtgacgccaatcgctcctactcggacgaggaccagagctcatccaacatagaagagtttgataagctccaggaaggcctggacagcagcggagatcccaagctctgcccaatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctgtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacaccctgtgcttccgcgtccgcaagatgatcgacgtctataagcccgactggtgcgaggtccgcgaagactggtctgtctacctcttctctcccgagaacaggttccgggtcctgtgtcag

SEQ ID NO: 14 - DNA, 3751 bp, Human
cgtccctccagcccgccgcctcctcctcctcccgccatggcccgggcccccgggcccgccgccccccgcccgccgccccccgcgccccgggggctcagctgatcctgaattggcggggggtcggctggcggggctgggggtccctctcgatcgccggcttcgggtggggacgcgttgtccccgcgtctctaacctggacgaccccccgcggccgggccacgtccatgccaagggctcccagctcagtgtggacatggctgagagcgcctccccgccctcctcatctgcagcagccccagccgctgagccaggagtcaccacggagcagcccggaccccggagccccccatcctccccgccaggcctggaggagcctctggatggagctgatcctcatgtcccacacccagacctggcgcctattgccttcttctgcctgcgacagaccaccagcccccggaactggtgcatcaagatggtgtgcaacccgtggtttgaatgtgtcagcatgctggtgatcctgctgaactgcgtgacacttggcatgtaccagccgtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcagggatggtcgagtactccctggaccttcagaacatcaacctgtcagccatccgcaccgtgcgcgtcctgaggcccctcaaagccatcaaccgcgtgcccagtatgcggatcctggtgaacctgctcctggacacactgcccatgctggggaatgtcctgctgctctgcttctttgtcttcttcatctttggcatcataggtgtgcagctctgggcgggcctgctgcgtaaccgctgcttcctggaggagaacttcaccatacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcaactttgacaacatcggttatgcttggattgtcatcttccaggtgatcactctggaaggctgggtggagatcatgtactacgtgatggatgctcactccttctacaacttcatctacttcatcctgcttatcatagtgggctccttcttcatgatcaacctgtgcctcgttgtcatagcgacccagttctcggagaccaagcaacgggagcaccggctgatgctggagcagcggcagcgctacctgtcctccagcacggtggccagctacgccgagcctggcgactgctacgaggagatcttccagtatgtctgccacatcctgcgcaaggccaagcgccgcgccctgggcctctaccaggccctgcagagccggcgccaggccctgggcccggaggccccggcccccgccaaacctgggccccacgccaaggagccccggcactaccagctgtgcccgcaacatagccccctggatgcgacgccccacaccctggtgcagcccatccccgccacgctggcttccgatcccgccagctgcccttgctgccagcatgaggacggccggcggccctcgggcctgggcagcaccgactcgggccaggagggctcgggctccgggagctccgctggtggcgaggacgaggcggatggggacggggcccggagcagcgaggacggagcctcctcagaactggggaaggaggaggaggaggaggagcaggcggatggggcggtctggctgtgcggggatgtgtggcgggagacgcgagccaagctgcgcggcatcgtggacagcaagtacttcaaccggggcatcatgatggccatcctggtcaacaccgtcagcatgggcatcgagcaccacgagcagccggaggagctgaccaacatcctggagatctgcaatgtggtcttcaccagcatgtttgccctggagatgatcctgaagctggctgcatttgggctcttcgactacctgcgtaacccctacaacatcttcgacagcatcattgtcatcatcagcatctgggagatcgtggggcaggcggacggtgggctgtcggtgctgcggaccttccggctgctgcgcgtgctgaaactggtgcgcttcatgcctgccctgcggcgccagctcgtggtgctcatgaagaccatggacaacgtggccaccttctgcatgctgctcatgctcttcatcttcatcttcagcatccttgggatgcatatttttggctgcaagttcagcctccgcacggacactggagacacggtgcccgacaggaagaacttcgactccctgctgtgggccatcgtcactgtgttccagatcctcacccaggaggactggaacgtcgttctctacaatggcatggcctccacttctccctgggcctccctctactttgtcgccctcatgaccttcggcaactatgtgctcttcaacctgctggtggccatcctggtggagggcttccaggcggagggtgacgccaatcgctcctactcggacgaggaccagagctcatccaacatagaagagtttgataagctccaggaaggcctggacagcagcggagatcccaagctctgcccaatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctgtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacaccctgtgcttccgcgtccgcaagatgatcgacgtctataagcccgactggtgcgaggtccgcgaagactggtctgtctacctcttctctcccgagaacaggttccgggtcctgtgtcagaccattattgcccacaaactcttcgactacgtcgtcctggccttcatctttctcaactgcatcaccatcgccctggagcggcctcagatcgaggccggcagcaccgaacgc

SEQ ID NO: 15 - DNA, 1205 bp, Human
caatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctgtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacaccctgtgcttccgcgtccgcaagatgatcgacgtctataagcccgactggtgcgaggtccgcgaagactggtctgtctacctcttctctcccgagaacaggttccgggtcctgtgtcagaccattattgcccacaaactcttcgactacgtcgtcctggccttcatctttctcaactgcatcaccatcgccctggagcggcctcagatcgaggccggcagcaccgaacgcatctttctcaccgtgtccaactacatcttcacggccatcttcgtgggcgagatgacattgaaggtagtctcgctgggcctgtacttcggcgagcaggcgtacctacgcagcagctggaacgtgctggatggctttcttgtcttcgtgtccatcatcgacatcgtggtgtccctggcctcagccgggggagccaagatcttgggggtcctccgagtcttgcggctcctgcgcaccctacgccccctgcgtgtcatcagccgggcgccgggcctgaagctggtggtggagacactcatctcctccctcaagcccatc

SEQ ID NO: 16 - DNA, 1385 bp, Human
gtgggcgagatgacattgaaggtagtctcgctgggcctgtacttcggcgagcaggcgtacctacgcagcagctggaacgtgctggatggctttcttgtcttcgtgtccatcatcgacatcgtggtgtccctggcctcagccgggggagccaagatcttgggggtcctccgagtcttgcggctcctgcgcaccctacgccccctgcgtgtcatcagccgggcgccgggcctgaagctggtggtggagacactcatctcctccctcaagcccatcggcaacatcgtgctcatctgctgtgccttcttcatcatctttggcatcctgggagtgcagctcttcaagggcaagttctaccactgtctgggcgtggacacccgcaacatcaccaaccgctcggactgcatggccgccaactaccgctgggtccatcacaaatacaacttcgacaacctgggccaggctctgatgtccctctttgtcctggcatccaaggatggttgggtgaacatcatgtacaatggactggatgctgttgctgtggaccagcagcctgtgaccaaccacaacccctggatgctgctgtacttcatctccttcctgctcatcgtcagcttctttgtgctcaacatgtttgtgggtgtcgtggtggagaacttccacaagtgccggcagcaccaggaggctgaagaggcacggcggcgtgaggagaagcggctgcggcgcctggagaagaagcgccggaaggcccagcggctgccctactatgccacctattgtcacacccggctgctcatccactccatgtgcaccagccactacctggacatcttcatcaccttcatcatctgcctcaacgtggtcaccatgtccctggagcactacaatcagcccacgtccctggagacagccctcaagtactgcaactatatgttcaccactgtctttgtgctggaggctgtgctgaagctggtggcatttggtctgaggcgcttcttcaaggaccgatggaaccagctggacctggccattgtgctactgtcagtcatgggcatcaccctggaggagatcgagatcaatgcggccctgcccatcaatcccaccatcatccgcatcatgagggttctgcgcattgcccgagtgctgaagctgttgaagatggccacaggaatgcgggccctgctggacacggtggtgcaagctttgccccaggtgggcaacctgggcctcctcttcatgctgctcttcttcatctatgctgctctcggggtggagctctttgggaagctggtctgcaacgacgagaacccgtgcgagggcatgagccggcatgccaccttcgagaacttcggcatggccttcctcacactcttccaggtctccacggg

SEQ ID NO: 17 - DNA, 1794 bp, Human
gaccgatggaaccagctggacctggccattgtgctactgtcagtcatgggcatcaccctggaggagatcgagatcaatgcggccctgcccatcaatcccaccatcatccgcatcatgagggttctgcgcattgcccgagtgctgaagctgttgaagatggccacaggaatgcgggccctgctggacacggtggtgcaagctttgccccaggtgggcaacctgggcctcctcttcatgctgctcttcttcatctatgctgctctcggggtggagctctttgggaagctggtctgcaacgacgagaacccgtgcgagggcatgagccggcatgccaccttcgagaacttcggcatggccttcctcacactcttccaggtctccacgggtgacaactggaacgggatcatgaaggacacgctgcgggactgcacccacgacgagcgcagctgcctgagcagcctgcagtttgtgtcgccgctgtacttcgtgagcttcgtgctcaccgcgcagttcgtgctcatcaacgtggtggtggctgtgctcatgaagcacctggacgacagcaacaaggaggcgcaggaggacgccgagatggatgccgagctcgagctggagatggcccatggcctgggccctggcccgaggctgcctaccggctccccgggcgcccctggccgagggccgggaggggcgggcggcgggggcgacaccgagggcggcttgtgccggcgctgctactcgcctgcccaggagaacctgtggctggacagcgtctctttaatcatcaaggactccttggagggggagctgaccatcatcgacaacctgtcgggctccatcttccaccactactcctcgcctgccggctgcaagaagtgtcaccacgacaagcaagaggtgcagctggctgagacggaggccttctccctgaactcagacaggtcctcgtccatcctgctgggtgacgacctgagtctcgaggaccccacagcctgcccacctggccgcaaagacagcaagggtgagctggacccacctgagcccatgcgtgtgggagacctgggcgaatgcttcttccccttgtcctctacggccgtctcgccggatccagagaacttcctgtgtgagatggaggagatcccattcaaccctgtccggtcctggctgaaacatgacagcagtcaagcacccccaagtcccttctccccggatgcctccagccctctcctgcccatgccagccgagttcttccaccctgcagtgtctgccagccagaaaggcccagaaaagggcactggcactggaaccctccccaagattgcgctgcagggctcctgggcatctctgcggtcaccaagggtcaactgtaccctcctccggcaggtaccgacacctcccaggccctagagcactggtctgtgggcaaggggcaggatctaagccaggcctggaagtccaagggactgggaggggaaggacccaaccaaaggccgagggcaccaccgtgcaagggggtttgggaacgctggggtgacgctgagactggagggggaggtggcactggggcggatggagtgggcggggctgggtcctggggacagcagagtgtggggaggaccccaaggcgggtctggaagaggcctgtgatccctagcttgaggggaggggaggagaggaggaggagtactggaggttttgcagggtggcggggtgctggcagtggggaggacaccctgggtgctctgggtgggtgtgagtgggggcttgattactaggaatggaggtgggagggcg

SEQ ID NO: 18 - DNA, 6745 bp, Human
gcagagccgaggcaggcgagagaccggcctcagccccgcgctccccgccgcgctggtctctcccgggcacttgccccgctgctcggtccccgcagcacccgcgcccccgccccggcccggagcgccgcgctcccgcccgccgcccgcccagagcctcgccgtccctccagcccgccgcctcctcctcctcccgccatggcccgggcccccgggcccgccgccccccgcccgccgccccccgcgccccgggggctcagctgatcctgaattggcggggggtcggctggcggggctgggggtccctctcgatcgccggcttcgggtggggacgcgttgtccccgcgtctctaacctggacgaccccccgcggccgggccacgtccatgccaagggctcccagctcagtgtggacatggctgagagcgcctccccgccctcctcatctgcagcagccccagccgctgagccaggagtcaccacggagcagcccggaccccggagccccccatcctccccgccaggcctggaggagcctctggatggagctgatcctcatgtcccacacccagacctggcgcctattgccttcttctgcctgcgacagaccaccagcccccggaactggtgcatcaagatggtgtgcaacccgtggtttgaatgtgtcagcatgctggtgatcctgctgaactgcgtgacacttggcatgtaccagccgtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcagggatggtcgagtactccctggaccttcagaacatcaacctgtcagccatccgcaccgtgcgcgtcctgaggcccctcaaagccatcaaccgcgtgcccagtatgcggatcctggtgaacctgctcctggacacactgcccatgctggggaatgtcctgctgctctgcttctttgtcttcttcatctttggcatcataggtgtgcagctctgggcgggcctgctgcgtaaccgctgcttcctggaggagaacttcaccatacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcaactttgacaacatcggttatgcttggattgtcatcttccaggtgatcactctggaaggctgggtggagatcatgtactacgtgatggatgctcactccttctacaacttcatctacttcatcctgcttatcatagtgggctccttcttcatgatcaacctgtgcctcgttgtcatagcgacccagttctcggagaccaagcaacgggagcaccggctgatgctggagcagcggcagcgctacctgtcctccagcacggtggccagctacgccgagcctggcgactgctacgaggagatcttccagtatgtctgccacatcctgcgcaaggccaagcgccgcgccctgggcctctaccaggccctgcagagccggcgccaggccctgggcccggaggccccggcccccgccaaacctgggccccacgccaaggagccccggcactaccagctgtgcccgcaacatagccccctggatgcgacgccccacaccctggtgcagcccatccccgccacgctggcttccgatcccgccagctgcccttgctgccagcatgaggacggccggcggccctcgggcctgggcagcaccgactcgggccaggagggctcgggctccgggagctccgctggtggcgaggacgaggcggatggggacggggcccggagcagcgaggacggagcctcctcagaactggggaaggaggaggaggaggaggagcaggcggatggggcggtctggctgtgcggggatgtgtggcgggagacgcgagccaagctgcgcggcatcgtggacagcaagtacttcaaccggggcatcatgatggccatcctggtcaacaccgtcagcatgggcatcgagcaccacgagcagccggaggagctgaccaacatcctggagatctgcaatgtggtcttcaccagcatgtttgccctggagatgatcctgaagctggctgcatttgggctcttcgactacctgcgtaacccctacaacatcttcgacagcatcattgtcatcatcagcatctgggagatcgtggggcaggcggacggtgggctgtcggtgctgcggaccttccggctgctgcgcgtgctgaaactggtgcgcttcatgcctgccctgcggcgccagctcgtggtgctcatgaagaccatggacaacgtggccaccttctgcatgctgctcatgctcttcatcttcatcttcagcatccttgggatgcatatttttggctgcaagttcagcctccgcacggacactggagacacggtgcccgacaggaagaacttcgactccctgctgtgggccatcgtcactgtgttccagatcctcacccaggaggactggaacgtcgttctctacaatggcatggcctccacttctccctgggcctccctctactttgtcgccctcatgaccttcggcaactatgtgctcttcaacctgctggtggccatcctggtggagggcttccaggcggagggtgacgccaatcgctcctactcggacgaggaccagagctcatccaacatagaagagtttgataagctccaggaaggcctggacagcagcggagatcccaagctctgcccaatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctgtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacaccctgtgcttccgcgtccgcaagatgatcgacgtctataagcccgactggtgcgaggtccgcgaagactggtctgtctacctcttctctcccgagaacaggttccgggtcctgtgtcagaccattattgcccacaaactcttcgactacgtcgtcctggccttcatctttctcaactgcatcaccatcgccctggagcggcctcagatcgaggccggcagcaccgaacgcatctttctcaccgtgtccaactacatcttcacggccatcttcgtgggcgagatgacattgaaggtagtctcgctgggcctgtacttcggcgagcaggcgtacctacgcagcagctggaacgtgctggatggctttcttgtcttcgtgtccatcatcgacatcgtggtgtccctggcctcagccgggggagccaagatcttgggggtcctccgagtcttgcggctcctgcgcaccctacgccccctgcgtgtcatcagccgggcgccgggcctgaagctggtggtggagacactcatctcctccctcaagcccatcggcaacatcgtgctcatctgctgtgccttcttcatcatctttggcatcctgggagtgcagctcttcaagggcaagttctaccactgtctgggcgtggacacccgcaacatcaccaaccgctcggactgcatggccgccaactaccgctgggtccatcacaaatacaacttcgacaacctgggccaggctctgatgtccctctttgtcctggcatccaaggatggttgggtgaacatcatgtacaatggactggatgctgttgctgtggaccagcagcctgtgaccaaccacaacccctggatgctgctgtacttcatctccttcctgctcatcgtcagcttctttgtgctcaacatgtttgtgggtgtcgtggtggagaacttccacaagtgccggcagcaccaggaggctgaagaggcacggcggcgtgaggagaagcggctgcggcgcctggagaagaagcgccggaaggcccagcggctgccctactatgccacctattgtcacacccggctgctcatccactccatgtgcaccagccactacctggacatcttcatcaccttcatcatctgcctcaacgtggtcaccatgtccctggagcactacaatcagcccacgtccctggagacagccctcaagtactgcaactatatgttcaccactgtctttgtgctggaggctgtgctgaagctggtggcatttggtctgaggcgcttcttcaaggaccgatggaaccagctggacctggccattgtgctactgtcagtcatgggcatcaccctggaggagatcgagatcaatgcggccctgcccatcaatcccaccatcatccgcatcatgagggttctgcgcattgcccgagtgctgaagctgttgaagatggccacaggaatgcgggccctgctggacacggtggtgcaagctttgccccaggtgggcaacctgggcctcctcttcatgctgctcttcttcatctatgctgctctcggggtggagctctttgggaagctggtctgcaacgacgagaacccgtgcgagggcatgagccggcatgccaccttcgagaacttcggcatggccttcctcacactcttccaggtctccacgggtgacaactggaacgggatcatgaaggacacgctgcgggactgcacccacgacgagcgcagctgcctgagcagcctgcagtttgtgtcgccgctgtacttcgtgagcttcgtgctcaccgcgcagttcgtgctcatcaacgtggtggtggctgtgctcatgaagcacctggacgacagcaacaaggaggcgcaggaggacgccgagatggatgccgagctcgagctggagatggcccatggcctgggccctggcccgaggctgcctaccggctccccgggcgcccctggccgagggccgggaggggcgggcggcgggggcgacaccgagggcggcttgtgccggcgctgctactcgcctgcccaggagaacctgtggctggacagcgtctctttaatcatcaaggactccttggagggggagctgaccatcatcgacaacctgtcgggctccatcttccaccactactcctcgcctgccggctgcaagaagtgtcaccacgacaagcaagaggtgcagctggctgagacggaggccttctccctgaactcagacaggtcctcgtccatcctgctgggtgacgacctgagtctcgaggaccccacagcctgcccacctggccgcaaagacagcaagggtgagctggacccacctgagcccatgcgtgtgggagacctgggcgaatgcttcttccccttgtcctctacggccgtctcgccggatccagagaacttcctgtgtgagatggaggagatcccattcaaccctgtccggtcctggctgaaacatgacagcagtcaagcacccccaagtcccttctccccggatgcctccagccctctcctgcccatgccagccgagttcttccaccctgcagtgtctgccagccagaaaggcccagaaaagggcactggcactggaaccctccccaagattgcgctgcagggctcctgggcatctctgcggtcaccaagggtcaactgtaccctcctccggcaggtaccgacacctcccaggccctagagcactggtctgtgggcaaggggcaggatctaagccaggcctggaagtccaagggactgggaggggaaggacccaaccaaaggccgagggcaccaccgtgcaagggggtttgggaacgctggggtgacgctgagactggagggggaggtggcactggggcggatggagtgggcggggctgggtcctggggacagcagagtgtggggaggaccccaaggcgggtctggaagaggcctgtgatccctagcttgaggggaggggaggagaggaggaggagtactggaggttttgcagggtggcggggtgctggcagtggggaggacaccctgggtgctctgggtgggtgtgagtgggggcttgattactaggaatggaggtgggagggcg

SEQ ID NO: 19 - protein, 1981 aa, Human [sequence omitted]

SEQ ID NO: 20 - DNA, 6799 bp, Human
gcagagccgaggcaggcgagagaccggcctcagccccgcgctccccgccgcgctggtctctcccgggcacttgccccgctgctcggtccccgcagcacccgcgcccccgccccggcccggagcgccgcgctcccgcccgccgcccgcccagagcctcgccgtccctccagcccgccgcctcctcctcctcccgccatggcccgggcccccgggcccgccgccccccgcccgccgccccccgcgccccgggggctcagctgatcctgaattggcggggggtcggctggcggggctgggggtccctctcgatcgccggcttcgggtggggacgcgttgtccccgcgtctctaacctggacgaccccccgcggccgggccacgtccatgccaagggctcccagctcagtgtggacatggctgagagcgcctccccgccctcctcatctgcagcagccccagccgctgagccaggagtcaccacggagcagcccggaccccggagccccccatcctccccgccaggcctggaggagcctctggatggagctgatcctcatgtcccacacccagacctggcgcctattgccttcttctgcctgcgacagaccaccagcccccggaactggtgcatcaagatggtgtgcaacccgtggtttgaatgtgtcagcatgctggtgatcctgctgaactgcgtgacacttggcatgtaccagccgtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcagggatggtcgagtactccctggaccttcagaacatcaacctgtcagccatccgcaccgtgcgcgtcctgaggcccctcaaagccatcaaccgcgtgcccagtatgcggatcctggtgaacctgctcctggacacactgcccatgctggggaatgtcctgctgctctgcttctttgtcttcttcatctttggcatcataggtgtgcagctctgggcgggcctgctgcgtaaccgctgcttcctggaggagaacttcaccatacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcaactttgacaacatcggttatgcttggattgtcatcttccaggtgatcactctggaaggctgggtggagatcatgtactacgtgatggatgctcactccttctacaacttcatctacttcatcctgcttatcatagtgggctccttcttcatgatcaacctgtgcctcgttgtcatagcgacccagttctcggagaccaagcaacgggagcaccggctgatgctggagcagcggcagcgctacctgtcctccagcacggtggccagctacgccgagcctggcgactgctacgaggagatcttccagtatgtctgccacatcctgcgcaaggccaagcgccgcgccctgggcctctaccaggccctgcagagccggcgccaggccctgggcccggaggccccggcccccgccaaacctgggccccacgccaaggagccccggcactaccagctgtgcccgcaacatagccccctggatgcgacgccccacaccctggtgcagcccatccccgccacgctggcttccgatcccgccagctgcccttgctgccagcatgaggacggccggcggccctcgggcctgggcagcaccgactcgggccaggagggctcgggctccgggagctccgctggtggcgaggacgaggcggatggggacggggcccggagcagcgaggacggagcctcctcagaactggggaaggaggaggaggaggaggagcaggcggatggggcggtctggctgtgcggggatgtgtggcgggagacgcgagccaagctgcgcggcatcgtggacagcaagtacttcaaccggggcatcatgatggccatcctggtcaacaccgtcagcatgggcatcgagcaccacgagcagccggaggagctgaccaacatcctggagatctgcaatgtggtcttcaccagcatgtttgccctggagatgatcctgaagctggctgcatttgggctcttcgactacctgcgtaacccctacaacatcttcgacagcatcattgtcatcatcaggcctcctactgctgcctcctacctgtaccctgggcctgccctgcgggaccgcagcatctgggagatcgtggggcaggcggacggtgggctgtcggtgctgcggaccttccggctgctgcgcgtgctgaaactggtgcgcttcatgcctgccctgcggcgccagctcgtggtgctcatgaagaccatggacaacgtggccaccttctgcatgctgctcatgctcttcatcttcatcttcagcatccttgggatgcatatttttggctgcaagttcagcctccgcacggacactggagacacggtgcccgacaggaagaacttcgactccctgctgtgggccatcgtcactgtgttccagatcctcacccaggaggactggaacgtcgttctctacaatggcatggcctccacttctccctgggcctccctctactttgtcgccctcatgaccttcggcaactatgtgctcttcaacctgctggtggccatcctggtggagggcttccaggcggagggtgacgccaatcgctcctactcggacgaggaccagagctcatccaacatagaagagtttgataagctccaggaaggcctggacagcagcggagatcccaagctctgcccaatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctgtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacaccctgtgcttccgcgtccgcaagatgatcgacgtctataagcccgactggtgcgaggtccgcgaagactggtctgtctacctcttctctcccgagaacaggttccgggtcctgtgtcagaccattattgcccacaaactcttcgactacgtcgtcctggccttcatctttctcaactgcatcaccatcgccctggagcggcctcagatcgaggccggcagcaccgaacgcatctttctcaccgtgtccaactacatcttcacggccatcttcgtgggcgagatgacattgaaggtagtctcgctgggcctgtacttcggcgagcaggcgtacctacgcagcagctggaacgtgctggatggctttcttgtcttcgtgtccatcatcgacatcgtggtgtccctggcctcagccgggggagccaagatcttgggggtcctccgagtcttgcggctcctgcgcaccctacgccccctgcgtgtcatcagccgggcgccgggcctgaagctggtggtggagacactcatctcctccctcaagcccatcggcaacatcgtgctcatctgctgtgccttcttcatcatctttggcatcctgggagtgcagctcttcaagggcaagttctaccactgtctgggcgtggacacccgcaacatcaccaaccgctcggactgcatggccgccaactaccgctgggtccatcacaaatacaacttcgacaacctgggccaggctctgatgtccctctttgtcctggcatccaaggatggttgggtgaacatcatgtacaatggactggatgctgttgctgtggaccagcagcctgtgaccaaccacaacccctggatgctgctgtacttcatctccttcctgctcatcgtcagcttctttgtgctcaacatgtttgtgggtgtcgtggtggagaacttccacaagtgccggcagcaccaggaggctgaagaggcacggcggcgtgaggagaagcggctgcggcgcctggagaagaagcgccggaaggcccagcggctgccctactatgccacctattgtcacacccggctgctcatccactccatgtgcaccagccactacctggacatcttcatcaccttcatcatctgcctcaacgtggtcaccatgtccctggagcactacaatcagcccacgtccctggagacagccctcaagtactgcaactatatgttcaccactgtctttgtgctggaggctgtgctgaagctggtggcatttggtctgaggcgcttcttcaaggaccgatggaaccagctggacctggccattgtgctactgtcagtcatgggcatcaccctggaggagatcgagatcaatgcggccctgcccatcaatcccaccatcatccgcatcatgagggttctgcgcattgcccgagtgctgaagctgttgaagatggccacaggaatgcgggccctgctggacacggtggtgcaagctttgccccaggtgggcaacctgggcctcctcttcatgctgctcttcttcatctatgctgctctcggggtggagctctttgggaagctggtctgcaacgacgagaacccgtgcgagggcatgagccggcatgccaccttcgagaacttcggcatggccttcctcacactcttccaggtctccacgggtgacaactggaacgggatcatgaaggacacgctgcgggactgcacccacgacgagcgcagctgcctgagcagcctgcagtttgtgtcgccgctgtacttcgtgagcttcgtgctcaccgcgcagttcgtgctcatcaacgtggtggtggctgtgctcatgaagcacctggacgacagcaacaaggaggcgcaggaggacgccgagatggatgccgagctcgagctggagatggcccatggcctgggccctggcccgaggctgcctaccggctccccgggcgcccctggccgagggccgggaggggcgggcggcgggggcgacaccgagggcggcttgtgccggcgctgctactcgcctgcccaggagaacctgtggctggacagcgtctctttaatcatcaaggactccttggagggggagctgaccatcatcgacaacctgtcgggctccatcttccaccactactcctcgcctgccggctgcaagaagtgtcaccacgacaagcaagaggtgcagctggctgagacggaggccttctccctgaactcagacaggtcctcgtccatcctgctgggtgacgacctgagtctcgaggaccccacagcctgcccacctggccgcaaagacagcaagggtgagctggacccacctgagcccatgcgtgtgggagacctgggcgaatgcttcttccccttgtcctctacggccgtctcgccggatccagagaacttcctgtgtgagatggaggagatcccattcaaccctgtccggtcctggctgaaacatgacagcagtcaagcacccccaagtcccttctccccggatgcctccagccctctcctgcccatgccagccgagttcttccaccctgcagtgtctgccagccagaaaggcccagaaaagggcactggcactggaaccctccccaagattgcgctgcagggctcctgggcatctctgcggtcaccaagggtcaactgtaccctcctccggcaggtaccgacacctcccaggccctagagcactggtctgtgggcaaggggcaggatctaagccaggcctggaagtccaagggactgggaggggaaggacccaaccaaaggccgagggcaccaccgtgcaagggggtttgggaacgctggggtgacgctgagactggagggggaggtggcactggggcggatggagtgggcggggctgggtcctggggacagcagagtgtggggaggaccccaaggcgggtctggaagaggcctgtgatccctagcttgaggggaggggaggagaggaggaggagtactggaggttttgcagggtggcggggtgctggcagtggggaggacaccctgggtgctctgggtgggtgtgagtgggggcttgattactaggaatggaggtgggagggcg

SEQ ID NO: 21 - protein, 1999 aa, Human
MAESASPPSSSAAAPAAEPGVTTEQPGPRSPPSSPPGLEEPLDGADPHVPHPDLAPIAFFCLRQTTSPRNWCIKMVCNPWFECVSMLVILLNCVTLGMYQPCDDMDCLSDRCKILQVFDDFIFIFFAMEMVLKMVALGIFGKKCYLGDTWNRLDFFIVMAGMVEYSLDLQNINLSAIRTVRVLRPLKAINRVPSMRILVNLLLDTLPMLGNVLLLCFFVFFIFGIIGVQLWAGLLRNRCFLEENFTIQGDVALPPYYQPEEDDEMPFICSLSGDNGIMGCHEIPPLKEQGRECCLSKDDVYDFGAGRQDLNASGLCVNWNRYYNVCRTGSANPHKGAINFDNIGYAWIVIFQVITLEGWVEIMYYVMDAHSFYNFIYFILLIIVGSFFMINLCLVVIATQFSETKQREHRLMLEQRQRYLSSSTVASYAEPGDCYEEIFQYVCHILRKAKRRALGLYQALQSRRQALGPEAPAPAKPGPHAKEPRHYQLCPQHSPLDATPHTLVQPIPATLASDPASCPCCQHEDGRRPSGLGSTDSGQEGSGSGSSAGGEDEADGDGARSSEDGASSELGKEEEEEEQADGAVWLCGDVWRETRAKLRGIVDSKYFNRGIMMAILVNTVSMGIEHHEQPEELTNILEICNVVFTSMFALEMILKLAAFGLFDYLRNPYNIFDSIIVIIRPPTAASYLYPGPALRDRSIWEIVGQADGGLSVLRTFRLLRVLKLVRFMPALRRQLVVLMKTMDNVATFCMLLMLFIFIFSILGMHIFGCKFSLRTDTGDTVPDRKNFDSLLWAIVTVFQILTQEDWNVVLYNGMASTSPWASLYFVALMTFGNYVLFNLLVAILVEGFQAEGDANRSYSDEDQSSSNIEEFDKLQEGLDSSGDPKLCPIPMTPNGHLDPSLPLGGHLGPAGAAGPAPRLSLQPDPMLVALGSRKSSVMSLGRMSYDQRSLSSSRSSYYGPWGRSAAWASRRSSWNSLKHKPPSAEHESLLSAERGGGARVCEVAADEGPPRAAPLHTPHAHHVHHGPHLAHRHRHHRRTLSLDNRDSVDLAELVPAVGAHPRAAWRAAGPAPGHEDCNGRMPSIAKDVFTKMGDRGDRGEDEEEIDYTLCFRVRKMIDVYKPDWCEVREDWSVYLFSPENRFRVLCQTIIAHKLFDYVVLAFIFLNCITIALERPQIEAGSTERIFLTVSNYIFTAIFVGEMTLKVVSLGLYFGEQAYLRSSWNVLDGFLVFVSIIDIVVSLASAGGAKILGVLRVLRLLRTLRPLRVISRAPGLKLVVETLISSLKPIGNIVLICCAFFIIFGILGVQLFKGKFYHCLGVDTRNITNRSDCMAANYRWVHHKYNFDNLGQALMSLFVLASKDGWVNIMYNGLDAVAVDQQPVTNHNPWMLLYFISFLLIVSFFVLNMFVGVVVENFHKCRQHQEAEEARRREEKRLRRLEKKRRKAQRLPYYATYCHTRLLIHSMCTSHYLDIFITFIICLNVVTMSLEHYNQPTSLETALKYCNYMFTTVFVLEAVLKLVAFGLRRFFKDRWNQLDLAIVLLSVMGITLEEIEINAALPINPTIIRIMRVLRIARVLKLLKMATGMRALLDTVVQALPQVGNLGLLFMLLFFIYAALGVELFGKLVCNDENPCEGMSRHATFENFGMAFLTLFQVSTGDNWNGIMKDTLRDCTHDERSCLSSLQFVSPLYFVSFVLTAQFVLINVVVAVLMKHLDDSNKEAQEDAEMDAELELEMAHGLGPGPRLPTGSPGAPGRGPGGAGGGGDTEGGLCRRCYSPAQENLWLDSVSLIIKDSLEGELTIIDNLSGSIFHHYSSPAGCKKCHHDKQEVQLAETEAFSLNSDRSSSILLGDDLSLEDPTACPPGRKDSKGELDPPEPMRVGDLGECFFPLSSTAVSPDPENFLCEMEEIPFNPVRSWLKHDSSQAPPSPFSPDASSPLLPMPAEFFHPAVSASQKGPEKGTGTGTLPKIALQGSWASLRSPRVNCTLLRQVPTPPRP

(19) United States
(12) Patent Application Publication
(10) Pub. No.: US 2008/0248040 A1
(43) Pub. Date: Oct. 9, 2008

(54) Nucleic Acid Molecules Encoding Novel Human Low-Voltage Activated Calcium Channel Proteins, Designed-Alpha 1I-1 and Alpha 1I-2, Encoded Proteins and Methods of Use Thereof

(76) Inventors: Menghang Xia, Blue Bell, PA (US); Mark E. Williams, Carlsbad, CA (US)

Correspondence Address: MERCK AND CO., INC, P O BOX 2000, RAHWAY, NJ, 07065-0907, US

(21) Appl. No.: 10/573,764

(22) PCT Filed: Sep. 28, 2004
(86) PCT No.: PCT/US2004/031820

§ 371 Date: Mar. 29, 2006

Related U.S. Application Data
(60) Provisional application No. 60/508,112, filed on Oct. 2, 2003.

(51) Int. Cl. A61K 39/395; C12N 15/11; C07K 14/00; C12N 15/00; A61K 31/70; G01N 33/569; C12P 21/04; A61P 43/00; C12Q 1/68; C07K 16/18; C12Q 1/02; C12N 5/06; G01N 33/00; G01N 33/53

(52) U.S. Cl. 424/139.1; 536/23.5; 530/350; 435/320.1; 435/325; 436/86; 435/7.8; 435/7.1; 514/44; 435/29; 435/7.21; 530/387.9; 435/366; 435/69.1; 435/6

(57) ABSTRACT

Disclosed are mammalian nucleic acid sequences encoding α1I subunit isoforms of a voltage-gated calcium channel. Specifically disclosed are novel variants of the α1I subunit designated herein as α1I-1 and α1I-2. In other aspects, the disclosure relates to expression vectors which encode the novel subunits of the invention, as well as cells containing such vectors. Antibodies specific for each of the variant subunits are also provided. The nucleic acid sequences of the invention find application, for example, in screening for compounds which modulate the activity of voltage-gated calcium channels and also in diagnostic methods for diagnosing various T-type channel mediated disorders, e.g., epilepsy, cancer, pain, sleep disorders and the autoimmune disease Lambert-Eaton Syndrome. Diagnosing defects in α1I subunit genes of a patient with a neuronal disease such as epilepsy are also included. An additional application of the nucleic acid sequences of the invention is in therapeutic methods of treatment for α1I subunit mediated disorders.

### BACKGROUND OF THE INVENTION

The present invention relates to novel nucleic acid molecules, encoded proteins, vectors, host cells transformed therewith, antibodies reactive with said proteins, as well as pharmaceutical compositions. Methods of using any of the foregoing, e.g., methods for screening for candidate agonists or antagonists utilizing the novel protein isoforms are also contemplated by the present invention.

Calcium is an essential signaling molecule for many normal physiological functions in the human body. These include all electrical signaling in the nervous system, as well as controlling heart and smooth muscle contraction, and hormone release. The entry of calcium into cells is regulated by a diverse set of proteins called calcium channels.

Calcium channels were discovered in 1958 by Fatt and Ginsborg when they explored the ionic basis of a Na⁺-independent action potential in crab muscle. The most unique and crucial role of Ca²⁺ channels is to translate the electrical signal on the surface membrane into a chemical signal within the cytoplasm, which, in general, increases the intracellular second messenger Ca²⁺, which, in turn, activates many crucial intracellular processes including contraction, secretion, neurotransmission and regulation of enzymatic activities and gene expression. Tsien et al., (1988), Trends Neurosci., vol. 11, pp. 431-438. As might be expected from their central role in signal transduction, Ca²⁺ channels are tightly regulated by a range of signal transduction pathways in addition to regulation by their intrinsic, voltage-dependent gating processes.

Continuing studies have revealed that there are multiple types of Ca²⁺ currents as defined by physiological and pharmacological criteria. See, e.g., Catterall, W. A., (2000) Annu. Rev. Cell Dev. Biol., 16:521-55; Llinas et al, (1992) Trends Neurosci, 15; 351-55; Hess, P. (1990) Ann. Rev. Neurosci. 56:337; Bean, B. P. (1989) Ann. Rev. Physiol. 51:367-384; and Tsien et al. (1988) Trends Neurosci. 11:431-38. In addition to exhibiting distinct kinetic properties, different Ca²⁺ channel types can be localized on different regions of a cell with complex morphology. Finally, Ca²⁺ channels in different tissues display different pharmacological profiles, suggesting the possibility of drugs selective for particular organs.

The calcium in nerve cells plays an important role in delivering signals between nerve cells. Calcium has many different delivery paths, however, when delivering peripheral stimuli, the voltage-activated calcium channel is crucial. Voltage activated channels play important roles including neuroexcitation, neurotransmitter and hormone secretion, and regulation of gene transcription through Ca-dependent transcription factors. Their functions depend in part on their cellular localization and their gating properties (characteristics of their opening, inactivation, deactivation, and recovery from inactivation). Five general classes of voltage activated calcium channels have been observed in various neuronal and non-neuronal tissues. The complement of calcium subunits and the subcellular localization of the expressed voltage activated calcium channels determine the functional cellular properties.

Native calcium channels have been classified by their electrophysiological and pharmacological properties as T, L, N, P and Q types (for views see McCleskey, E. W. et al. Curr Topics Membr (1991) 39:295-326, and Dunlap, K. et al. Trends Neurosci (1995) 18:89-98). Voltage-gated calcium channels can be divided into Low Voltage Activated calcium channel (LVA) that is activated at a lower voltage and High Voltage Activated (HVA) calcium channel that is activated at a higher voltage than the resting membrane potential. HVA channels are currently known to comprise at least three groups of channels, known as L-, N- and P/Q-type channels. These channels have been distinguished from one another electrophysiologically as well as biochemically on the basis of their pharmacology and ligand binding properties. The L, N, P and Q-type channels activate at more positive potentials (high voltage activated) and display diverse kinetics and voltage-dependent properties. A fourth type of high voltage-activated calcium channel (Q-type) has been described, although whether the Q- and P-type channels are distinct molecular entities is controversial (Sather, W. A et al. Neuron (1995) 11:291-303; Stea, A. et al. Proc Natl Acad Sci USA (1994) 91:10576-10580; Bourinet, E. et al. Nature Neuroscience (1999) 2:407-415).

To date, only one type of low-threshold calcium channel is known, the T-type calcium channel. These channels are so called because they carry a transient current with a low voltage of activation and rapid inactivation. (Ertel and Ertel (1997) Trends Pharmacol. Sci. 18:37-42.) In general, T-type calcium channels are involved in the generation of low threshold spikes to produce burst firing (Huguenard, 1996). The main factor which defines the different calcium currents is which al subtype is included in the channel complex. The subfamily of α_{1G}, α_{1H} and α_{1I} subunits display the low-voltage activation characteristic of T-type channels.

Structurally, most Ca²⁺ channels are complexes of a pore-forming α1 subunit of ca ˜190-250 kDa; a transmembrane, disulfide-linked complex of α2 and δ subunits; and an intracellular β subunit. See, e.g., De Waard et al. Structural and functional diversity of voltage-activated calcium channels. In Ion Channels, (ed. T. Narahashi) 41-87, (Plenum Press, New York, 1996); Catterall, W. A., (1991a), Cell, Vol. 64, pp. 871-874; and Catterall, W. A., (1991b), Science, vol. 253, pp. 1499-1500. The al subunit contains the binding sites for selective channel antagonists and is composed of four homologous repeats (I-IV) each comprising six transmembrane segments (S1-S6). Three genes have been identified for the α₁ subunits of LVA channels, reviewed in Hofmann et al., (1999), Rev. Physiol. Biochem. Phamacol. 139:33-87; Lacinova et al., (2000) Gen. Physiol. Biophys., 19: 121-36).

The surface of voltage gated calcium channels comprises molecular pores that “open” in response to the depolarization of the membrane voltage, which, in turn, allows for the selective influx of Ca²⁺ ions from an extracellular environment into the interior of a cell. The “opening” of the pores essentially requires a depolarization to a certain level of the potential difference between the inside of the cell bearing the channel and the extracellular medium bathing the cell. The rate of influx of Ca²⁺ into the cell depends on this potential difference. When the accumulating Ca²⁺ reaches a sufficient concentration, it can activate ion channels such as Ca²⁺ activated K⁺ channels that allow positive charge out the cell and thereby repolarize the membrane. It can be seen how calcium channels serve as elements that can sense, amplify, and terminate electrical signals.

T-type channels are located in cardiac & vascular smooth muscle; and in the nervous system. Perez-Reyes et al. discuss the molecular characterization of a neuronal low-voltage-activated T-type calcium channel (Nature 391, 896-900, 1998. Generally, T-type channels are thought to be involved in pacemaker activity, low-threshold calcium spikes, neuronal oscillations and resonance, and rebound burst firing. See F. R. Buhler, J. Hypertension supplement 15(5):s3-7, 1997; B. Cremers et al., J. Cardiovascular Pharmacology, vol. 29(5), pp. 692-6, 1997. The functional roles for T-type calcium channels in neurons include, inter alia, membrane depolarization, calcium entry and burst firing. (White et al. (1989) Proc. Natl. Acad. Sci. USA 86:6802-6806.) The LVA channels differ from HVA channels in a number of ways, i.e., length of I-II intracellular linker etc and the β subunit does not appear to be associated with α1 in the LVA class. As well, they lack the canonical sequence that is known to be crucial for beta subunit binding (Lambert et al., J. Neurosci., 17; 6621-6628, 1997; Leuranguer et al., Neuropharmacology, 37: 701-708, 1998.

The physiological roles and potential therapeutic areas pertaining to T-type channels are numerous. For example, changes to calcium influx into neuronal cells may be implicated in conditions such as epilepsy, stroke, brain trauma, Alzheimer's disease, multiinfarct dementia, other classes of dementia, Korsakoff's disease, neuropathy caused by a viral infection of the brain or spinal cord (e.g., human immunodeficiency viruses, etc.), amyotrophic lateral sclerosis, convulsions, seizures, Huntington's disease, amnesia, pain transmission, cardiac pacemaker activity or damage to the nervous system resulting from reduced oxygen supply, poison or other toxic substances (See e.g., Goldin et al., U.S. Pat. No. 5,312,928). Other pathological conditions associated with elevated intracellular free calcium levels include muscular dystrophy and hypertension (Steinhardt et al., U.S. Pat. No. 5,559,004).

Indeed, changes to calcium influx into cardiovascular cells may be implicated in conditions such as cardiac arrhythmia, angina pectoris, hypoxic damage to the cardiovascular system, ischemic damage to the cardiovascular system, myocardial infarction, and congestive heart failure (Goldin et al., supra). More, T-type calcium channels have been implicated in cellular growth and proliferation, particularly in the cardiovascular system (Katz, A. M, Eur. Heart J. Suppl., H18-H23, 999; Lijnen and Petrov, Exp. Clin. Pharmacol., 21: 253-259, 1999; Richard and Nargeot, Electrophsiol. Meet., 123-132, 1998; Wang et al., Am. J. Physiol. 265: C1239-C1246, 1993. Of equal import is the observation that there is limited knowledge in the art of the role of calcium channel types in cell growth control and abnormalities of calcium channels leading to cancer development.

As an example, the low threshold spikes and rebound burst firing characteristic of T-type calcium currents is prominent in neurons from inferior olive, thalamus, hippocampus, lateral habenular cells, dorsal horn neurons, sensory neurons (DRG, no dose), cholinergic forebrain neurons, hippocampal intraneurons, CA1, CA3 dentate gyrus pyramidal cells, basal forebrain neurons, amygdaloid neurons (Talley et al., J. Neurosci., 19: 1895-1911, 1999) and neurons in the thalamus. (Suzuki and Rogawski, Proc. Natl. Acad. Sci. USA 86:7228-7232, 1998). As well, T-type channels are prominent in the soma and dendrites of neurons that reveal robust Ca-dependent burst firing behaviors such as the thalamic relay neurons and cerebellar Purkinje cells (Huguenard, J. R., Annu. Rev. Physiol., 329-348, 1996. Consequently, improper functioning of these LVA channels has been implicated in arrhythmias, chronic peripheral pain, improper pain transmission in the central nervous system to name a few.

For example, the data show that T-type channels promote oscillatory behavior which has important consequences for epilepsy. The ability of a cell to fire low threshold spikes is critical in the genesis of oscillatory behavior and increased burst firing (groups of action potentials separated by about 50-100 ms). T-type calcium channels are believed to play a vital role in absence epilepsy, a type of generalized non-convulsive seizure. The evidence that voltage-gated calcium currents contribute to the epileptogenic discharge, including seizure maintenance and propagation includes 1) a specific enhancement of T-type currents in the reticular thalamic (nRT) neurons which are hypothesized to be involved in the genesis of epileptic seizures in a rat genetic model (GAERS) for absence epilepsy (Tsakiridou et al., J. Neurosci., 15: 3110-3117, 1995); 2) antiepileptics against absence petit mal epilepsy (ethosuximide and dimethadione) have been shown at physiologically relevant doses to partially depress T-type currents in thalamic (ventrobasal complex) neurons (Coulter et al., Ann. Neurol., 25:582-93, 1989; U.S. Pat. No. 6,358,706 and references cited therein); and 3) T-type calcium channels underlie the intrinsic bursting properties of particular neurons that are hypothesized to be involved in epilepsy (nRT, thalamic relay and hippocampal pyramidal cells) (Huguenard, supra). The rat α_{1G} is highly expressed in thalamocortical relay cells (TCs) which are capable of generating prominent Ca²⁺-dependent low-threshold spikes (Talley et al., J. Neurosci., 19: 1895-1911, 1999).

The T-type calcium channels have also been implicated in thalamic oscillations and cortical synchrony, and their involvement has been directly implicated in the generation of cortical spike waves that are thought to underlie absence epilepsy and the onset of sleep (McCormick and Bal, Annu. Rev. Neurosci., 20: 185-215, 1997). Oscillations of neural networks are critical in normal brain function such during sleep-wave cycles. It is widely recognized that the thalamus is intimately involved in cortical rhythmogenesis. Thalamic neurons most frequently exhibit tonic firing (regularly spaced spontaneous firing) in awake animals, whereas phasic burst firing is typical of slow-wave sleep and may account for the accompanying spindling in the cortical EEG. The shift to burst firing occurs as a result of activation of a low threshold Ca²⁺ spike which is stimulated by synaptically mediated inhibition (i.e., activated upon hyperpolarization of the RP). The reciprocal connections between pyramidal neurons in deeper layers of the neocortex, cortical relay neurons in the thalamus, and their respective inhibitory interneurons are believed to form the elementary pacemaking circuit. That anti-epileptic drugs cause a reduction of the low-threshold calcium current (LTCC or T-type Ca²⁺ current) in thalamic neurons is evident from the prior art. See Coulter et al. (1989) Ann. Neurol. 25:582-593.) For example, ethosuximide, an anti-epileptic drug has been shown to fully block T-type Ca²⁺ current in freshly dissected neurons from dorsal root ganglia (DRG neurons) of adult rats (Todorovic and Lingle, J. Neurophysiol. 79:240-252, 1998), and may have limited efficacy in the treatment of abnormal, chronic pain syndromes that follow peripheral nerve damage.

T-type channels have also been implicated as contributing to spontaneous fluctuations in intracellular calcium concentrations [Ca]i. Changes to calcium influx into cardiovascular cells, in turn, may be implicated in conditions such as cardiac arrhythmia, angina pectoris, hypoxic damage to the cardiovascular system, ischemic damage to the cardiovascular system, myocardial infarction, and congestive heart failure (Goldin et al., supra). Other pathological disease states associated with dysfunctional calcium channels, e.g., elevated intracellular free calcium levels include muscular dystrophy and hypertension (Steinhardt et al., U.S. Pat. No. 5,559,004). Consequently, T-type calcium channels are important in pacemaker activity and therefore heart rate in the heart, and in vesicle release from non-excitable cells (Ertel et al. In cardiovasc. Drugs Ther., 723-739, 1997). It is believed that therapeutic moieties capable of blocking the T-type channel in specific conformational states will find use in the treatment of tachycardia (by decreasing the heart rate) while having little effect on the inotropic properties of the normal heart. See Rousseau et al., J. Am. Coll. Cardiol., 28: 972-979, 1996. According to Sen and Smith, Circ. Res., 75: 149-55, 1994, in a particular cardiomyopathic disease (genetic Syrian hamster model), the disease status results from Ca-overload due to an increased expression of T-type calcium channels in ventricular myocytes. Likewise, researchers have shown that there are increased T-type currents in atrial myocytes from adult rats with growth hormone-secreting tumors. See also Xu and Best, Proc. Natl. Acad. Sci. U.S.A., 87: 4655-4659, 1990; U.S. Pat. No. 6,358,706 and references cited therein. Consequently, a specific T-type calcium channel blocker would find use as a cardioprotectant in these cases.

It is well documented that cortisol is the precursor for glucocorticoids and prolonged exposure to glucocorticoids causes breakdown of peripheral tissue protein, increased glucose production by the liver and mobilization of lipid from the fat depots. Furthermore, individuals suffering from anxiety and stress produce abnormally high levels of glucocorticoids. Consequently, drugs that would regulate these levels would aid in the treatment of stress disorders, e.g., antagonists to CRF. In this regard, the observations of Enyeart et al., Mol. Endocrinol., 7:1031-1040, 1993, that T-type channels in adrenal zona fasciculata cells of the adrenal cortex modulate cortisol secretion will greatly aid in the identification of such a therapeutic candidate.

T-type calcium channels may also be involved in release of nutrients from testis Sertoli cells. Sertoli cells are testicular cells that are thought to play a major role in sperm production. Sertoli cells secrete a number of proteins including transport proteins, hormones and growth factors, enzymes which regulate germinal cell development and other biological processes related to reproduction (Griswold, Int. Rev. Cytol., 133-156, 1988). They secrete the peptide hormone inhibin B, an important negative feedback signal to the anterior pituitary. They assist in spermiation (the final detachment of the mature spermatozoa from the Sertoli cell into the lumen) by releasing plasminogen activator which produces proteolytic enzymes. The data show that T-type calcium channels are expressed on immature rat Sertoli cells according to Lalevee et al., 1997. The intimate juxtaposition of the developing germ cells with the Sertoli cells suggests that the Sertoli cells may indeed pay a role in supporting and nurturing the gametes. While the role of T-type calcium channels is not well documented, it is believed that they may be important in the release of nutrients, inhibin B, and/or plasminogen activator and thus may impact sperm production. According to researchers, the inhibition of T-type calcium channels in sperm during gamete interaction inhibits zona pellucida-dependent Ca²⁺ elevations and inhibits acrosome reactions, thus directly linking sperm T-type calcium channels to fertilization. See Arnoult et al., 1996.

Likewise, tremor can be controlled through the basal ganglia and the thalamus, regions in which T type calcium channels are strongly expressed (Talley et al., supra). T-type calcium channels have been implicated in the pathophysiology of tremor since the anti-epileptic drug ethosuximide is used for treating tremor, in particular, tremor associated with Parkinson's disease, essential tremor, or cerebellar disease (U.S. Pat. No. 4,981,867; D. A. Prince).

T-type calcium channels also facilitate insulin secretion by enhancing the general excitability of these cells. Therefore, T-type calcium channels may be therapeutic targets in hypo- and hyperinsulinemia (A. Bhattacharjee et al., Endocrinology, vol. 138(9), pp. 3735-40, 1997). A direct link between T-type calcium channel activity and steroidogenesis has been suggested (M. F. Rossier et al., 1996).

Cellular calcium homeostasis plays an essential part in the physiology of nerve cells. The intracellular calcium concentration is about 0.1 uM compared with 1 mM outside the nerve cell. This steep concentration gradient (×10,000) is regulated primarily by voltage-gated calcium channels. Several pathologies of the central nervous system involve damage to or inappropriate function of voltage-gated calcium channels. In cerebral ischaemia (stroke) the channels of neurons are kept in the open state by prolonged membrane depolarization, producing a massive influx of calcium ions. This, in turn activates various calcium/calmodulin dependent cellular enzyme systems, e.g. kinases, proteases and phospholipases. Such prolonged activation leads to irreversible damage to nerve cells.

Certain diseases, such as Lambert-Eaton Syndrome, involve autoimmune interactions with calcium channels. The availability of the calcium channel subunits makes possible immunoassays for the diagnosis of such diseases. An understanding of them at the molecular level will lead to effective methods of treatment.

As well, there is a need for a better understanding of the structure and function of calcium channels, which, in turn would permit identification of substances that, in some manner, modulate the activity of calcium channels and that have potential for use in treating such disorders. That mutations of several channel proteins have been shown to be a causative factor in neurological disorders, is well known, thereby making the calcium channel subunits target for therapeutic interventions. See, e.g., Marais, supra and Burgess and Noebels, (1999) Epilepsy Res., 36:111-122.

An understanding of the pharmacology of compounds that interact with calcium channels in other organ systems, such as the central nervous system (“CNS”), will greatly aid in the rational design of compounds that specifically interact with the specific subtypes of human calcium channels to have desired therapeutic effects, such as in the treatment of neurodegenerative and cardiovascular disorders. Such an understanding together with the ability to rationally design therapeutically effective compounds have been hampered by an inability to independently determine the types of human calcium channels and the molecular nature of individual subtypes, particularly in the CNS, and by the unavailability of pure preparations of specific channel subtypes to use for evaluation of the specificity of calcium channel-effecting compounds. Thus, the identification of nucleic acid molecules encoding human calcium channel subunits coupled with the use of such molecules for expression of the encoded calcium channel subunits subsequent use in of the functional calcium channels would aid in screening and design of therapeutically effective compounds.

A number of compounds useful in treating various diseases in animals, including humans, are thought to exert their beneficial effects by modulating functions of voltage-gated calcium channels. Many of these compounds bind to calcium channels and block, or reduce the rate of influx of calcium into cells in response to depolarization of the inside and outside of the cells. An understanding of the pharmacology of compounds that interact with calcium channels, and the ability to rationally design compounds that will interact with calcium channels to have desired therapeutic effects, depends upon the understanding of the structure of calcium subunits and the genes that encode them. The identification and study of tissue specific subunits allows for the development of therapeutic compounds specific for pathologies of those tissues.

However, there is a paucity of understanding of the pharmacology of compounds which interact with calcium channels. This paucity of understanding, together with the limited knowledge in the art of the human calcium channel types, the molecular nature of the human calcium channel subtypes, and the limited availability of pure preparations of specific calcium channel subtypes to use for evaluating the efficacy of calcium channel-modulating compounds has hampered the rational testing and screening of compounds that interact with the specific subtypes of human calcium channels to have desired therapeutic effects.

While a number of pharmacological blockers have differential effects on T type calcium currents expressed in different cell types as noted supra, there are no known specific blockers of the T-type class of calcium channel. It is believed that the differential sensitivity of T-type currents to antagonists may be due to different subunit structure (Perez-Reyes, 1998) as well as cellular environments. T-type calcium channel alpha subunit genes, like the genes for HVA channels, reveal alternative splicing (Lee et al., 1999 Biophys J 76:A408). Extracellular and intracellular loops of individual T-type calcium channel clones also show marked diversity amongst themselves and even less homology to HVA channels.

Examples of conventional putative calcium channel blockers include dihydropyridines such as nifedipine, nitrendipine, nicardipine, nimodipine, niludipine, riodipine (ryosidine) felodipine, darodipine, isradipine, (+)Bay K 8644, (−)202-791, (+)H 160/S1, PN 200-110 and nisoldipine. Other examples of the calcium channel blocker include Kurtoxin, benzothiazepine, such as diltiazem (dilzem) and TA 3090 and phenylalkylamine, such as verapamil (isoptin), desmethoxyverapamil, methoxy verapamil (D-600, gallopamil or (−)D-888), prenylamine, fendiline, terodiline, caroverine, perhexyline.

In view of the above, pharmacological modulation of T-type calcium channels' function is very important and therapeutic moieties capable of modulating T-type currents will find tremendous use in the practice of medicine, i.e., calcium channel blockers for the treatment of epilepsy, hypertension, and angina pectoris etc. Unfortunately, as noted above, conventional medicine and its use of conventional calcium channels blockers for the treatment of a wide variety of calcium channels mediated diseases is not very effective. Importantly, such intervention is not yet available for calcium channels in electrically non-excitable cells. This deficiency likely reflects the fact that the mechanism by which calcium entry occurs has not been clearly identified.

Consequently, the discovery of the herein disclosed two isoforms of the human α_{1I} subunit will allow for the development of therapeutic compounds specific for pathologies mediated by the low voltage all subunit of the T-type calcium channel and/or characterized by aberrant voltage regulated calcium influx thereby satisfying a long-sought need for such therapies and tools. Sequence and structural homology exists between the novel all isoforms detailed herein. Significantly, the development of new therapeutic strategies against, and the creation of new analytical tools for a better understanding of diseases characterized by aberrant voltage regulated calcium influx are greatly desired.

Accordingly, in view of the importance of specific calcium channels in neuronal function, it would be useful to identify pharmacological agents that specifically inhibit or potentiate the class T calcium channel. The present invention meets these needs.

It is particularly desirable to provide tools and therapies that are highly specific to a target gene and protein, which at the same time, because of their specificity, do not substantially affect other proteins or body functions. The present invention meets these needs by detailing the discovery of human T-type calcium channel α_{1I} isoforms that are useful in diagnosis of disease states associated with the peripheral nervous system and for screening compounds that may be used in the treatment of mammals for various disease states mediated by T-type calcium channel currents and responsive to modulators of α_{1I} of the T-type channels.

### SUMMARY OF THE INVENTION

The present invention is based on the discovery of two mammalian nucleic acid sequences encoding alternatively spliced human T-type calcium channel α_{1I} subunits of a human T-type calcium channel. Specifically disclosed are two T-type calcium channel α_{1I} subunits designated herein as α_{1I-1} and α_{1I-2}, nucleic acid molecules encoding each of the disclosed variants together with uses thereof.

One of the two sequences, α_{1I-1} differs by a single nucleotide from the reference sequence, resulting in a change from isoleucine to valine, which is thus a single nucleotide polymorphic sequence, resulting from the phenomenon known as Single Nucleotide Polymorphism (SNP). The other nucleic acid molecule designated α_{1I-2}, is a naturally occurring sequence obtained by alternative splicing of the known α_{1I} genes, designated herein as the reference sequence—GeneBank #AF211189. Thus, the α_{1I-1} results from a single nucleotide polymorphism of the reference sequence while α_{1I-2} results from alternative splicing of the reference sequence.

The novel variant encoding sequence(s) of the invention are not merely truncated forms or fragments of the known gene, but rather novel sequences, which naturally occur within the body of individuals and may thus have physiological relevance. The inventors have discovered that one of the herein disclosed nucleotide sequences encoding one of the α_{1I} calcium channel subunit isoforms (variant #1, SEQ ID NO: 19 differs from the reference sequence in at least one (1) amino acid, e.g., at position 1005, specifically Isoleucine (I) at position 1005 of the reference sequence is valine in the novel isoform of the invention—vis-à-vis I 1005>V 1005. The other variant (isoform-#2) contains a 54 nucleotide insert (SEQ ID NO:20) encoding eighteen (18) amino acid residues relative to SEQ ID NOS:18 and 19 after amino acid 679 of SEQ ID NO:19.

The novel α_{1I} calcium channel subunit isoforms of the invention are meant to define distinct but closely related calcium channel proteins, i.e., those having an amino acid homology of approximately 99% with each other and sharing a significant, over 90% sequence homology with the reference α_{1I} subunit sequence. The novel isoforms are also thought to be functionally similar to the prior art α_{1I} subunit isoforms based upon the structural similarity between the isoforms of the invention and the reference protein.

In accordance with the above, there are provided isolated nucleic acid molecules, comprising a sequence of nucleotides that encodes a functional human T-type calcium channel α_{1I} subunit polypeptide or a molecule comprising a sequence having at least 90% identity to any one of SEQ ID NOS:18 or 20. Preferably, the isolated nucleic acid molecules comprise the nucleotide sequence as set forth in SEQ ID NOS:18 or 20, wherein SEQ ID NO:18 comprises a coding portion encoding variant #1 (SV#1 or α_{1I-1}), while SEQ ID NO:20 encodes splice variant #2 (SV#2 or α_{1I-2}).

An illustrative nucleic acid molecule containing a sequence that encodes the α_{1I-1} polypeptide has the nucleotide sequence of SEQ ID NO:18 of 5943 nucleotides. (SEQ ID NO:18 is 6745 nts long because it contains untranslated sequences. The coding sequence contained within SEQ ID 18 is 5943 nts). The encoded polypeptide has the amino acid sequence as set forth in SEQ ID NO:19. Thus, the α_{1I-1} nucleotide sequence described herein encodes a polypeptide of that is 1981 amino acids.

An illustrative nucleic acid molecule containing a sequence that encodes the α_{1I-2} polypeptide has the nucleotide sequence of SEQ ID NO: 20. The encoded polypeptide has the amino acid sequence as set forth in SEQ ID NO: 21. Thus, the α_{1I-2} nucleotide sequence described herein encodes a polypeptide of 1999 amino acids.

The prior art is replete with information detailing the therapeutic utilities in recombinant materials derived from the DNA of the numerous calcium channels including the various isoforms of the α_{1I} subunit.

Considering the high degree (>90%) of sequence homology in the primary sequence between the reference α_{1I} subunit isoform sequence and the novel isoform sequences disclosed herein, it is believed that compositions comprising the novel isoforms or a biologically fragment or derivative thereof may be administered to a subject to treat or prevent a pathological disorder characterized by a dysfunctional T-type calcium channel subunit mediated by an α_{1I} calcium channel isoform subunit. As such, the novel isoforms of the invention may find use, inter alia, in treating a number of α_{1I} subunit mediated pathologies including epilepsy, colorectal cancers, gastric cancers, acute myelogenous-leukemias as well as lung and breast cancers. See Gao et al., supra.

The invention further provides nucleic acid molecule(s) comprising a nucleotide sequence which is complementary to that of SEQ ID NO:18 or SEQ ID NO:20, or complementary to a sequence having at least 90% identity to said sequence or a fragment of said sequence. The complementary sequence may be a DNA sequence which hybridizes with any one of SEQ ID NOS:18 or 20 or hybridizes to a portion of that sequence having a length sufficient to inhibit the transcription of the complementary sequence. The complementary sequence may be a DNA sequence which can be transcribed into an mRNA being an antisense to the mRNA transcribed from any one of SEQ ID NOS:18 or 20 or into an mRNA which is an antisense to a fragment of the mRNA transcribed from any one of SEQ ID NOS:18 or 20 which has a length sufficient to hybridize with the mRNA transcribed from any one SEQ ID NOS:18 or 20, so as to inhibit its translation. The complementary sequence may also be the mRNA or the fragment of the mRNA itself.

The present invention further provides nucleic acid molecule comprising a nucleotide sequence which encode the amino acid sequences of SEQ ID NOS:19 or 21, including fragments and homologues of the amino acid sequences. Due to the degenerative nature of the genetic code, a plurality of alternative nucleic acid sequences beyond those depicted in any one of SEQ ID NOS:18 or 20, can code for the amino acid sequences of the invention. Consequently, those alternative nucleic acid sequences which code for the same amino acid sequences coded by the sequence of SEQ ID NO:18 or SEQ ID NO:20 are also included in the scope of the present invention.

Also provided, are expression vector constructs comprising the human T-type calcium channel α_{1I} subunit encoding nucleic acid molecules (SEQ ID NO:18 and SEQ ID NO:20) operably linked to a promoter. Host cells transformed or transfected with the expression vector are also within the scope of the invention.

The nucleic acid molecules detailed herein will find use in therapeutics or in diagnostic applications, e.g., as probes used for detecting genomic sequences comprising sequences substantially identical to those detailed herein or for detecting sequences encoding one of the novel isoforms detailed herein in a biological sample. Importantly, the presence of the α_{1I} encoding transcripts provided herein or the level of variant transcripts may be indicative of a multitude of diseases, disorders and various pathological conditions typically in connection with the diseases mediated by aberrant calcium currents specified above, as well as normal conditions. As well, the ratio of the level of the transcripts of any one of the novel isoforms of the invention may also be compared to that of the transcripts of the reference α_{1I} sequences from which it has been varied or compared to the level of other transcripts, and said ratio may be indicative of a multitude of diseases, disorders and various pathological and normal conditions as described above.

In another aspect, the invention provides a protein or polypeptide comprising an amino acid sequence encoded by any of the above nucleic acid sequences, termed herein “α_{1I} variant” or “protein isoform” or grammatical equivalents thereof. In one embodiment, the polypeptide corresponding to α_{1I-1} comprises the amino acid sequence of SEQ ID NO:19. In another embodiment the polypeptide corresponding to α_{1I}-2 (SV#2) comprises the amino acid sequence of SEQ ID NO:21. Fragments of the above amino acid sequences of sufficient length coded by the above fragments of the nucleic acid sequences, as well as homologues of the above amino acid sequences in which one or more of the amino acid residues has been substituted (by conservative or non-conservative substitution) added, deleted, or chemically modified are also within the scope of the invention.

The deletions, insertions and modifications should be in regions, or adjacent to regions, wherein the novel isoforms differs from the reference sequence, but maintains its ability to regulate voltage gated calcium influx. Applicants appreciate that a skilled artisan will be able to modify the novel isoforms or fragments thereof by addition, deletions or substitutions of amino acids ((derivative product/polypeptide). Consequently, homologues of the α_{1I} variants which are derivated from the reference α_{1I} isoforms e.g., α_{1I-1} (SEQ ID NO:19) or α_{1I-2} (SEQ ID NO:21) by changes (deletion, addition, substitution) are also a part of the present invention, wherein said derivatized protein is functionally equivalent to the novel isoforms detailed herein, i.e., ability to modulate voltage-gated calcium influx etc. In general, if the α_{1I} variants are distinguished from the reference α_{1I-1 or -2} sequence by some sort of physiological activity, then the homolog is distinguished from the reference α_{1I} sequence in essentially the same manner.

Another aspect of the invention features a dominant negative human T-type calcium channel corresponding to any one or both of the isoforms provided by the invention. The dominant negative polypeptide inhibits the function of the calcium channel.

Use of the foregoing compositions in the preparation of a medicament, and particularly in the preparation of a medicament for the treatment of α_{1I} mediated disorders including epilepsy, neuropathic pain, or a condition which results from excessive or insufficient voltage regulated calcium influx are also a part of the present invention.

The invention also provides anti-variant product antibodies, namely antibodies directed against the variant products of the invention, which specifically bind to said variant product(s). These antibodies are useful both for diagnostic and therapeutic purposes.

Consequently, an aspect of the invention features antibodies termed “distinguishing antibodies” which are directed solely to the amino acid sequences which distinguishes the α_{1I-1} variant from the other α_{1I-2} variant protein or from a reference α_{1I} amino acid sequence from which the variants have been varied either by alternative splicing or as a result of single nucleotide polymorphism. For example, these antibodies may be directed to alternative regions containing inserted new amino acid sequences. The regions may also be regions wherein a new sequence was obtained due to splicing. The distinguishing antibodies may be used for detection purposes, i.e. to detect individuals, tissue, conditions (both pathological or physiological) wherein at least one of the herein disclosed variant sequences or reference sequence are low or high (as compared to a normal control). The antibodies may also be used to distinguish conditions where the level, or ratio of the α_{1I} variants to a reference α₁ sequence or the ratio of one variant (SEQ ID NO:19) to the other variant (SEQ ID NO:21) or other variants have been varied. The distinguishing antibodies may also be used for therapeutical purposes, i.e., to neutralize only the α_{1I-1} subunit or the α_{1I-2} subunit or only the product of the reference α_{1I} sequence, as the case may be, without neutralizing the other.

“Variant” or “isoform” grammatical equivalents include not only the single nucleotide polymorphic sequence of SEQ ID NO:18 but also the splice variant of SEQ ID NO: 20. The present invention still further provides pharmaceutical compositions comprising, as an active ingredient, said nucleic acid molecules, said expression vectors, or said protein/polypeptide or anti-variant product antibodies will find use in the treatment of diseases and pathological conditions where a therapeutically beneficial effect may be achieved by neutralizing the variants of the invention (either at the transcript or product level) or decreasing the amount of the either of the variants of the invention or blocking its binding to its ligand (calcium etc) or the molecule it effects (G-protein), for example, by the neutralizing effect of the antibodies, or by decreasing the effect of the antisense mRNA in decreasing expression level of the α_{1I} subunit isoform of the invention. Typically, these diseases are of neurologic or psychiatric origin, e.g., neurodegenerative diseases, epilepsy, anxiety, schizophrenia, manic depression, depression, delirium, or for the treatment of cancer etc. In general, these are diseases wherein α_{1I} or other auxiliary subunit proteins of the calcium channel plays a role in the etiology of the disease, i.e. aberrant (excessive or insufficient voltage regulated calcium influx) cause or are a result of the disease.

Eukaryotic cells containing heterologous nucleic acids, such as SEQ ID NOS:18 or 20 encoding one or more calcium channel subunits, particularly human calcium channel subunits, i.e., SEQ ID NOS:19 or 21 or variants thereof or containing RNA transcripts of DNA clones encoding one or more of the subunits are also detailed herein.

A single α_{1I} subunit can form a functional channel. The requisite combination of subunits for formation of active channels in selected cells, however, can be determined empirically using the methods herein. For example, if a selected α_{1I}, subtype or variant does not form an active channel in a selected cell line, an additional subunit or subunits can be added until an active channel is formed. Other subunits can be added to assess the effects of such addition. In preferred embodiments, the cells contain DNA or RNA encoding an α_{1I} subunit, preferably an α_{1I} subunit of an animal, preferably of a mammalian calcium channel. Embodiments in which the cells contain nucleic acid encoding an α_{1I} subtype are of particular interest herein.

Thus, in one aspect, the invention provides one the first opportunity to use each of the major neuronal calcium channel subunit types together in a recombinant system for testing neuronal calcium channels. Consequently, an aspect of the invention provides for the use of a recombinant cell system as described elsewhere to develop functional calcium flux assays. Such assays can be used to screen for compounds which modulate the activity, as agonists or antagonists, of neuronal voltage-gated calcium channels. Several examples of such recombinant systems and screening assays have been described (e.g., Spreyer et al. U.S. Pat. No. 5,643,750; Jay et al., U.S. Pat. No. 5,726,035; and Jay et al. U.S. Pat. No. 5,386,025.) The disclosure of these patents are incorporated herein by reference. Potential agonists and antagonists of T-type calcium channels can be obtained from sources such as pre-existing therapeutic compounds, plant extracts, synthetic chemical libraries, and recombinant molecular libraries. The effectiveness of agonists and antagonists of calcium channels is expected to be of highest physiological relevance when each of the major subunit types α1, β, α2δ, and γ are present together in a test system. This is due to subunit interactions affecting the accessibility of target sites. As discussed above, additional molecules that contribute to the function or regulation of the calcium channel can also be included in the recombinant cell system in order to generate calcium channels that more accurately reflect the biological properties of calcium channels in vivo.

In other embodiments, the recombinant cells contain DNA or RNA encoding additional heterologous subunits, including the α_{1I} of the invention. The cells may further include nucleic acid encoding any other or additional subunits as noted above top study for example the various interactions of a functional calcium channel. In such embodiments, eukaryotic cells stably or transiently transfected with any combination of one, two, three or four of the subunit-encoding DNA clones, such as DNA encoding any of α, α₁A, β, α₂δ, and γ are provided. The eukaryotic cells provided herein contain heterologous nucleic acid that encodes an α₁ subunit and optionally a heterologous α₂δ, and or a γ subunit.

In preferred embodiments, the cells express such heterologous calcium channel subunits and include one or more of the subunits in membrane-spanning heterologous calcium channels. In more preferred embodiments, the eukaryotic cells express functional, heterologous calcium channels that are capable of gating the passage of calcium channel-selective ions and/or binding compounds that, at physiological concentrations, modulate the activity of the heterologous calcium channel. In certain embodiments, the heterologous calcium channels include at least one heterologous calcium channel subunit. In most preferred embodiments, the calcium channels that are expressed on the surface of the eukaryotic cells are composed substantially or entirely of subunits encoded by the heterologous DNA or RNA. In preferred embodiments, the heterologous calcium channels of such cells are distinguishable from any endogenous calcium channels of the host cell.

As such, the recombinant cells of the invention provide a means to obtain homogeneous populations of calcium channels. Typically, the cells contain the selected calcium channel as the only heterologous ion channel expressed by the cell. Preferably, the α₁ of the calcium channel is one of the disclosed subunits of the invention comprising the amino acid sequences as set forth in one of SEQ ID NOS:19 or 21.

In certain embodiments the recombinant eukaryotic cells that contain the heterologous DNA encoding the calcium channel subunits are produced by transfection with DNA encoding one or more of the subunits or are injected with RNA transcripts of DNA encoding one or more of the calcium channel subunits. The DNA may be introduced as a linear DNA fragment or may be included in an expression vector for stable or transient expression of the subunit-encoding DNA. Vectors containing DNA encoding human calcium channel subunits of the invention are also provided.

Eukaryotic cells expressing heterologous calcium channels may be used in assays for calcium channel function or, in the case of cells transformed with fewer subunit-encoding nucleic acids than necessary to constitute a functional recombinant human calcium channel, such cells may be used to assess the effects of additional subunits on calcium channel activity. The additional subunits can be provided by subsequently transfecting such a cell with one or more DNA clones or RNA transcripts encoding human calcium channel subunits.

The recombinant eukaryotic cells that express membrane spanning heterologous calcium channels may be used in methods for identifying compounds that modulate calcium channel activity. In particular, the cells are used in assays that identify agonists and antagonists of calcium channel activity in humans and/or assessing the contribution of the various calcium channel subunits to the transport and regulation of transport of calcium ions. Because the cells constitute homogeneous populations of calcium channels, they provide a means to identify agonists or antagonists of calcium channel activity that are specific for each such population.

The recombinant cells of the invention may be used to assess T-type channel function and tissue distribution and to identify compounds that modulate the activity of T-type channels. Because T-type channels are operative in neurons in the thalamus, hypothalamus, and brain stem, and may be involved in autonomic nervous functions, in regulation of cardiovascular activities such as heart rate, arterial and venous smooth muscle innervation and tone, pulmonary rate and other fundamental processes, assays designed to assess such activities and assays to identify modulators of these activities provides a means to understand fundamental physiological processes and also a means to identify new drug candidates for an array of disorders.

Assays that use the eukaryotic cells (“recombinant cells”) for identifying compounds that modulate calcium channel activity are also encompassed by the invention.

In practicing these assays the eukaryotic cell that expresses a heterologous calcium channel is in a solution containing a test compound and a calcium channel selective ion, the cell membrane is depolarized, and current flowing into the cell is detected. If the test compound is one that modulates calcium channel activity, the current that is detected is different from that produced by depolarizing the same or a substantially identical cell in the presence of the same calcium channel-selective ion but in the absence of the compound (control cell). Preferably, prior to the depolarization step, the cell is maintained at a holding potential which substantially inactivates calcium channels which are endogenous to the cell. As well, in certain preferred embodiments, the cells are mammalian cells, most preferably HEK cells, or amphibian oocytes.

In accordance with the above, the invention provides for a method for screening test compounds for modulating calcium channel activity, comprising: a) providing: i) the test compound; ii) a calcium channel selective ion; iii) a control cell; and iv) a host cell comprising a cell membrane and expressing heterologous nucleic acid sequences encoding: a functional calcium channel α₁ subunit; preferably one having the amino acid sequence as set forth in one of SEQ ID NOS:19 or 21 or a biologically equivalent/active fragment thereof; b) contacting the host cell with the test compound and with the molecule to produce a treated host cell; c) depolarizing the cell membrane of the treated host cell under conditions such that the molecule enters the cell through a functional calcium channel; and d) detecting a difference between current flowing into the treated host cell and current flowing into a control cell, thereby identifying the test compound as a compound capable of modulating calcium channel activity. In one preferred embodiment, the method further comprises, prior to the depolarizing, maintaining the treated host cell at a holding potential that substantially inactivates endogenous calcium channels. In another preferred embodiment, the method further comprises, prior to or simultaneously with the step of contacting the host cell with the test compound, contacting the host cell with a calcium channel agonist, wherein the test compound is tested for activity as an antagonist.

Cells that express T-channels or LVA channels may also be used in assays that screen for compounds that have activity as modulators, particularly antagonists, of the activity of these channels.

Thus, in accordance with the above, the invention provides a method for screening for compounds which modulate the activity of T-type voltage-gated calcium channels. The method involves providing a cell transformed with a DNA expression vector comprising a mammalian cDNA sequence encoding a T-type-specific α_{1I} subunit of a voltage-gated calcium channel, the cell comprising additional calcium channel subunits necessary and sufficient for assembly of a functional voltage-gated calcium channel. The cell is contacted with a test compound and agonistic or antagonistic action of the test compound on the reconstituted calcium channels is determined. Without intending to limit the type or source of host cell, in yet another preferred embodiment, the host cell is eukaryotic. In a more preferred embodiment, the eukaryotic host cell is selected from the group consisting of cancer cells and amphibian oocytes.

In alternative embodiments transcription based assays for identifying compounds that modulate the activity of calcium channels (see, U.S. Pat. Nos. 5,436,128 and 5,401,629), in particular calcium channels that contain an α_{1I} subunit, preferably those of the invention are also provided. The method proposes using cells that express calcium channels, particularly calcium channels containing an α_{1I} subunit, and more preferably an α_{1I} subunit encoded by heterologous DNA, and also contain nucleic acid encoding a reporter gene construct containing a reporter gene in operative linkage with one or more transcriptional control elements that is regulated by a calcium channel. The assays are effected by comparing the difference in the amount of transcription of a the reporter gene in the cells provided herein in the presence of the compound with the amount of transcription in the absence of the compound, or with the amount of transcription in the absence of the heterologous calcium channel, whereby compounds that modulate the activity of the heterologous calcium channel in the cell are identified. The reporter gene is any such gene known to those of skill in the art, including, but not limited to the gene encoding bacterial chloramphenicol acetyltransferase, the gene encoding firefly luciferase, the gene encoding bacterial luciferase, the gene encoding fl-galactosidase or the gene encoding alkaline phosphatase, and the transcriptional control element is any such element known to those of skill in the art, including, but not limited to serum responsive elements, cyclic adenosine monophosphate responsive elements, the c-fos gene promoter, the vasoactive intestinal peptide gene promoter, the somatostatin gene promoter, the proenkephalin promoter, the phosphoenolpyruvate carboxykinase gene promoter or the nerve growth factor-1 A gene promoter and elements responsive to intracellular calcium ion levels. In certain embodiments the heterologous nucleic acid encodes one or more of the herein disclosed novel α_{1I} subunit of SEQ ID NOS:18 or 20. Variants or biologically active fragments thereof are also included and encompassed by the assays disclosed herein.

Other reporter based assays may include the use of a dye which coordinate Ca²⁺. The method provides (i) incubating recombinant cells of the invention (those expressing a function calcium channel α_{1I} subunit) with (1) a dye which has acid groups which can coordinate Ca²⁺ and which undergoes a spectral shift when coordinated to Ca²⁺ and (2) a compound with unknown effect; (ii) stimulating Ca²⁺ influx into the cell; and (iii) monitoring the spectral characteristics of the dye in the recombinant cells. These spectral characteristics will change as calcium is bound to the dye. Because calcium will bind to (be coordinated by) the dye in proportion to the concentration of calcium in the activated cell, the change in spectral characteristics of the dye will be a measure of the calcium concentration within the cell. If the compound is a T-type channel selective inhibitor then the absorbance or fluorescent emission of the uncoordinated dye (A) will be different than the absorbance or fluorescent emission of the Ca²⁺-coordinated dye (A2) because the inhibitor will have suppressed calcium entry into the cell. In preferred embodiments, the DNA is one of SEQ ID NOS:18 or 20.

Other assays formats for identifying calcium channel modulators, in particular T-type calcium channels are also provided.

An exemplary assay proposes measuring receptor activity in response to a test compound using the recombinant cells provided herein (see, U.S. Pat. No. 5,670,113).

Because T-type channels appear to be associated with a variety of key functions, cells that express T-channels and assays using such cells will have utility in the identification of compounds for treatment of a variety of disorders, disease and conditions. Compounds identified thereby will be candidates for use in the treatment of disorders and conditions associated with T-channel activity. Such activities include, but are not limited to, those involving role in muscle excitability, secretion and pacemaker activity, Ca²⁺ dependent burst firing, neuronal oscillations, and potentiation of synaptic signals, for improving arterial compliance in systolic hypertension, or improving vascular tone, such as by decreasing vascular welling, in peripheral circulatory disease, and others. Other disorders include, but are not limited to hypertension, cardiovascular disorders, including but not limited to: myocardial infarct, cardiac arrhythmia, heart failure and angina pectoris; neurological disorders, such as schizophrenia, epilepsy and depression, peripheral muscle disorders, respiratory disorders and endocrine disorders.

In particular, cells that express LVA channels, such as the α_{1I} subunits preferably those encoding SEQ ID NOS:19 or 21, are useful for identifying compounds that are candidates for treatment of disorders associated with conduction tissues, such as atrial pacemaker cells, Purkinje fibers, and also coronary smooth muscles.

Such disorders include, but are not limited to, compounds useful for treatment of cardiovascular, such as angina, vascular, such as hypertension, and urologic, hepatic, reproductive, adjunctive therapies for reestablishing normal heart rate and cardiac output following traumatic injury, heart attack and other cardiac injuries; treatments of myocardial infarct (MI), post-Ml and in an acute setting. Other compounds that interact with LVA, particularly T-type, calcium channels, may be effective for increasing cardiac contractile force, such as measured by left ventricular end diastolic pressure, and without changing blood pressure or heart rate. Alternatively, other compounds may be effective to decrease formation of scar tissue, such as that measured by collagen deposition or septal thickness, and without cardiodepressant effects. The herein disclosed assays may be used to identify compounds useful in regulating vascular smooth muscle tone, either vasodilating or vasoconstricting in: (a) treatments for reestablishing blood pressure control, e.g., following traumatic injury, surgery or cardiopulmonary bypass, and in prophylactic treatments designed to minimize cardiovascular effects of anesthetic drugs; (b) treatments for improving vascular reflexes and blood pressure control by the autonomic nervous system; for identifying compounds useful in treating urological disorders: (a) treating and restoring renal function following surgery, traumatic injury, uremia and adverse drug reactions; (b) treating bladder dysfunctions; and (c) uremic neuronal toxicity and hypotension in patients on hemodialysis; reproductive disorders, for identifying compounds useful in treating: (a) disorders of sexual function including impotence; (b) alcoholic impotence (under autonomic control that may be subject to T-channel controls); hepatic disorders for identifying compounds useful in treating and reducing neuronal toxicity and autonomic nervous system damage resulting from acute over-consumption of alcohol; neurologic disorders for identifying compounds useful in treating: (a) epilepsy and diencephalic epilepsy; (b) Parkinson's disease; (c) aberrant temperature control, such as, abnormalities of shivering and sweat gland secretion and peripheral vascular blood supply; (d) aberrant pituitary and hypothalamic functions including abnormal secretion of noradrenalin, dopamine and other hormones; for respiratory such as in treating abnormal respiration, e.g., post-surgical complications of anesthetics; and endocrine disorders, for identifying compounds useful in treating aberrant secretion of hormones including e.g., possible treatments for overproduction of insulin, thyroxin, adrenalin, and other hormonal imbalances.

Also provided herein is a method of generating antibodies directed against at least a portion of a calcium channel protein, comprising: a) providing: i) at least a portion of an amino acid sequence selected from the group consisting of SEQ ID NO:19 and variants thereof, and SEQ ID NO:21 and variants thereof,; and ii) a host; b) immunizing the host with at least a portion of the amino acid sequence so as to generate an antibody; and c) collecting the antibody from the host. In one preferred embodiment, the method further comprises step d) purifying the antibody. Without limiting the type of host cell, in another preferred embodiment, the host is a mammal. In yet another preferred embodiment, the mammal is a mouse.

The immunoglobulins that are produced using the calcium channel subunits or purified calcium channels as immunogens have, among other properties, the ability to specifically and preferentially bind to and/or cause the immunoprecipitation of a human calcium channel or a subunit thereof which may be present in a biological sample or a solution derived from such a biological sample. Such antibodies may also be used to selectively isolate cells that express calcium channels that contain the subunit for which the antibodies are specific.

Also provided is a method of detecting expression of at least a portion of a calcium channel protein, comprising: a) providing: i) a sample suspected of expressing the calcium channel protein; and ii) an antibody raised according to the above-described method (i.e., by a) providing: i) at least a portion of an amino acid sequence selected from the group consisting of SEQ ID NO:19 and variants thereof, and SEQ ID NO:21 and variants thereof; and ii) a host; b) immunizing the host with at least a portion of the amino acid sequence so as to generate an antibody; and c) collecting the antibody from the host); b) combining the sample and the antibody under conditions such that the antibody binds to the calcium channel protein; and c) detecting the binding between the antibody and the calcium channel protein in the sample.

Methods for modulating the activity of ion channels by contacting the calcium channels with an effective amount of the above-described antibodies are also provided.

Consequently, in another aspect, assays for identifying compounds that modulate the activity of LVA calcium channels, particularly T-type channels are provided as well as compounds identified by the methods.

Methods for treating subjects suffering from or at risk of being afflicted with a pathology/disease characterized by aberrant voltage regulated calcium influx are also embraced by the invention. The disease status can be characterized as aberrant—excessive or insufficient voltage regulated calcium influx relative to normal.

Thus, an aspect of the invention features a method for selectively treating a subject having a condition characterized by aberrant brain neuronal calcium current. The method features administering to a subject in need of such treatment a pharmacological agent which is selective for a human T-type calcium channel α2δ subunit isoform, e.g., SEQ ID NO:19 or 21, in an amount effective to normalize the aberrant neuronal calcium current. By “aberrant” is meant a level of calcium current (calcium influx) which is outside of a normal range as understood in the medical arts. Normalize means that the calcium current is brought within the normal range.

Yet another aspect of the invention features methods for (i) detecting the level of the transcript (mRNA) of said α_{1I-1} or α_{1I-2} variants products (SEQ ID NO:18 or SEQ ID NO:20, including fragments thereof) in a body fluid sample, or in a specific tissue sample, for example by use of probes comprising all or parts of the nucleotide sequences disclosed herein; (ii) detecting levels of expression of said variant product in tissue, e.g. by the use of antibodies capable of specifically reacting with the gene products of the nucleotide sequences of the invention or biologically equivalent fragments thereof. Detection of the level of the expression of a variant product(s) of the invention in particular as compared to that of the reference sequence from which it was varied or compared to other variant sequences all varied from the same reference sequence may be indicative of a plurality of physiological or pathological conditions. Quantifying normal levels of the target gene or its encoded gene product are well known to a skilled artisan.

The method, according to this latter aspect, for detecting a nucleic acid sequence which encodes at least one of the human T-type calcium channel α_{1I} subunit isoforms in a biological sample, comprises the steps of:

(a) providing a probe comprising at least one of the nucleic acid sequences defined above;

(b) contacting the biological sample with said probe under conditions allowing hybridization of nucleic acid sequences thereby enabling formation of hybridization complexes;

(c) detecting hybridization complexes, wherein the presence of the complex indicates the presence of nucleic acid sequence encoding the α_{1I} subunit isoform in the biological sample.

The method as described above is qualitative, i.e. indicates whether the transcript is present in or absent from the sample. The method can also be quantitative, by determining the level of hybridization complexes and then calibrating said levels to determining levels of transcripts of the desired variant in the sample. Both qualitative and quantitative determination methods can be used for diagnostic, prognostic and therapy planning purposes.

The nucleic acid sequence used in the above method may be a DNA sequence, an RNA sequence, etc; it may be a coding or a sequence or a sequence complementary thereto (for respective detection of RNA transcripts or coding-DNA sequences). By quantization of the level of hybridization complexes and calibrating the quantified results it is possible also to detect the level of the transcript in the sample.

Methods for detecting mutations in the region coding for an α_{1I} variant product are also provided, which may be methods carried-out in a binary fashion, namely merely detecting whether there is any mismatches between the normal variant nucleic acid sequence of the invention and the one present in the sample, or carried-out by specifically detecting the nature and location of the mutation.

The present invention also concerns a method for detecting a human T-type calcium channel α_{1I} subunit isoform of the invention in a biological sample, comprising the steps of:

(a) contacting said biological sample with a detectable antibody of the invention, thereby forming an antibody-antigen complex; and

(b) detecting said antibody-antigen complex

wherein the presence of said antibody-antigen complex correlates with the presence of the α_{1I} isoform in the biological sample.

As indicated above, the method can be quantitized to determine the level or the amount of the α_{1I} subunit isoform in the sample, alone or in comparison to the level of the reference α_{1I} subunit amino acid sequence from which it was varied, and qualitative and quantitative results may be used for diagnostic, prognostic and therapy planning purposes.

Another aspect of the invention details a method for inhibiting human T-type calcium channel α_{1I} subunit isoform activity in a mammalian cell. The method proposes contacting the mammalian cell with an amount of a human T-type calcium channel α_{1I} subunit isoform inhibitor sufficient to inhibit calcium influx in the mammalian cell. Preferably the inhibitor is selected from the group consisting of a peptide or an antibody which selectively binds the respective α_{1I} subunit isoform, an antisense nucleic acid which binds a nucleic acid encoding the subject isoform, and a dominant negative human T-type calcium channel α_{1I} subunit isoform.

Yet another aspect of the invention details a method for treating a subject suspected of suffering from an α_{1I} mediated disorder-epilepsy, stroke, pain (e.g., neuropathic pain). The method proposes administering to a subject in need of such treatment an inhibitor of the human T-type calcium channel α_{1I} subunit polypeptide isoform in an amount effective to inhibit voltage regulated calcium influx. In another embodiment of the foregoing methods, the inhibitor is administered prophylactically to a subject at risk of having a stroke.

Utilizing a similar method, methods of increasing human T-type calcium channel α_{1I} subunit isoform expression in a cell are also provided. In certain embodiments, the cell may further be contacted with one or more human auxiliary T-type calcium channel non-α_{1I} subunits, such as a δ or γ subunit, or nucleic acid molecules encoding such non-α_{1I} subunits.

The invention further features a method for identifying a candidate pharmacological agent useful in the treatment of diseases associated with increased or decreased voltage regulated calcium influx mediated by a human T-type calcium channel α_{1I} subunit isoform of the invention. A cell comprising a human T-type calcium channel isoform, preferably one of the isoforms disclosed herein, vis-à-vis SEQ ID NOS:19 or 21 is loaded with a calcium-sensitive compound which is detectable in the presence of calcium. The cell is contacted with a candidate pharmacological agent under conditions which, in the absence of the candidate pharmacological agent, cause a first amount of voltage regulated calcium influx into the cell. A test amount of voltage regulated calcium influx then is determined. For example, in a preferred embodiment, fluorescence of a calcium-sensitive compound then is detected as a measure of the voltage regulated calcium influx. If the test amount of voltage regulated calcium influx is less than the first amount, then the candidate pharmacological agent is a lead compound for a pharmacological agent which reduces voltage regulated calcium influx. If the test amount of voltage regulated calcium influx is greater than the first amount, then the candidate pharmacological agent is a lead compound for a pharmacological agent which increases voltage regulated calcium influx.

In yet another aspect, the invention features an agent which selectively binds the human T-type calcium channel α_{1I} subunit isoforms or a nucleic acid that encodes the subject isoform. By “selectively binds” it is meant that the agent binds the human T-type calcium channel α_{1I} subunit isoform or nucleic acid encoding said isoform, or any fragment thereof to a greater extent than the agent binds other human α_{1I} subunit isoforms, and preferably does not bind other α_{1I} subunit isoforms. The agent may be a monoclonal antibody, a polyclonal antibody, or an antibody fragment selected from the group consisting of a Fab fragment, a F(ab)₂ fragment and a fragment including a CDR3 region. In another embodiment, the agent is an antisense nucleic acid which selectively binds to a nucleic acid encoding at least one the α_{1I} subunit isoforms disclosed herein It is preferred that the foregoing agents are inhibitors (antagonists) or agonists of the calcium channel activity of the subject α_{1I} subunit isoform. In other embodiments one may use the nucleotide sequence of SEQ ID NO:20 or the amino acid sequence of SEQ ID NO:21.

In another aspect of the invention, methods for identifying compounds which selectively or preferentially bind a human T-type calcium channel α_{1I} subunit isoform of the invention are provided. In one embodiment, the method proposes providing (i) a test cell preparation which expresses the subject α_{1I} subunit isoform of the invention, e.g., α_{1I} subunit, and providing a control cell preparation wherein said cell expresses a human T-type calcium channel subunit polypeptide other than the isoform in the test preparation, e.g., α1B or C isoform, (ii) contacting each preparation with a compound, and (iii) the determining binding of the compound to the test cell and said control cell preparation, wherein if the compound that binds the test cell preparation but does not bind the control cell preparation is considered a candidate compound which selectively binds the subject α_{1I-1} subunit isoform of the invention. Likewise, if the test compound binds the test cell preparation in an amount greater than the control cell preparation, it is a compound which preferentially binds the subject α_{1I-1} subunit isoform of the invention, e.g., SEQ ID NO:19.

By yet another aspect the invention also provides a method for identifying candidate compounds capable of binding to a human T-type calcium channel α_{1I} subunit isoform and modulating its activity (being either agonists or antagonists). The method comprises:

(i) providing a protein or polypeptide comprising an amino acid sequence substantially as depicted in any one of SEQ ID NO.:19 and SEQ ID NO.:21, or a fragment of such a sequence;

(ii) contacting a candidate compound with said amino acid sequence;

(iii) measuring the physiological effect of said candidate compound on the activity of the amino acid sequences and selecting those compounds which show a significant effect on said physiological activity.

Likewise, another aspect of the invention discloses a method for following the progress of a therapeutic regiment designed to alleviate a condition characterized by abnormal or aberrant expression of a gene product expressed from the isolated nucleic acid molecule having a sequence of nucleotides as set forth in SEQ ID NO:18 or 20 comprising:

(a) assaying a sample from a subject to determine level of a parameter selected from the group consisting of (i) a polypeptide encoded by the nucleotide sequence of SEQ ID NOS:18 or 20 and (ii) a polynucleotide encoding the amino acid sequence of SEQ ID NOS:19 or 21, at a first time point;

(b) assaying the level of the parameter selected in (a) at a second time point and

(c) comparing said level at said second time point to the level determined in (a) as a determination of effect of said therapeutic regime.

In another aspect, a method is provided for the treatment, inhibition or prevention of insufficient pain perception in a subject in need thereof. The method includes administering to the subject a pain sensitizing amount of an enhancer of T-type Ca²⁺ channel activity or a pharmaceutically acceptable salt or prodrug thereof to treat, inhibit or prevent insufficient pain perception in the subject.

Other embodiments provide methods for the potentiation and/or inhibition of a T-type Ca²⁺ channel in a subject in need of such potentiation/inhibition or in a tissue comprising a T-type Ca²⁺ channel. The method comprises administering to the subject or tissue either an amount of a reducing agent or of an oxidizing agent or a pharmaceutically acceptable salt or prodrug thereof effective either to potentiate or inhibit the T-type Ca²⁺ channel's activity as desired. In a further aspect of the invention a method of screening candidate pain perception modifying agents is provided. Such method comprises administering a candidate to an expression system comprising a T-type Ca²⁺ channel or its active fragment, derivative, analog or mimic and determining whether the channel, fragment, derivative, analog or mimic's activity is thereby modified.

Compounds identified by any of the herein disclosed methods are also within the scope of the invention.

Also included are methods for diagnosing LVA calcium channel-mediated, particularly T-type channel-mediated, disorders. Methods of diagnosis will involve detection of aberrant channel expression or function, such altered amino acid sequences, altered pharmacological profiles and altered electrophysiological profiles compared to normal or wild-type channels. Such methods typically can employ antibodies specific for the altered channel or nucleic acid probes to detect altered genes or transcripts.

These include, for example, a method of diagnosing Lambert-Eaton Syndrome and a method for diagnosing a defect in an α_{1I} subunit gene of a patient with a neuronal disease such as epilepsy. Therapeutic methods based on the disclosure are also described.

Methods of diagnosing Lambert-Eaton Syndrome may rely on the immunoreactivity towards the α_{1I} isoform protein of a patient suffering symptoms of Lambert-Eaton Syndrome, which can be determined to specifically diagnose the patient's autoimmune disorder. α_{1I} protein can be obtained by the methods described above. Purified α_{1I} protein can then be combined with antibody-containing serum from the patient to determine if the patient possesses autoimmunity towards α_{1I} protein.

In another aspect, the present invention relates to diagnostic screening techniques useful for the identification of mutations within the α_{1I} encoding (Caᵥ3.3) gene that is involved in neuronal disorders. Initial identification of mutations responsible for such conditions can be made, for example, by producing cDNA from the mRNA of an individual suffering from a neuronal disorder (e.g., epilepsy). The sequence of nucleotides in the cDNA is then determined by conventional techniques. This determined sequence is then compared to the wild-type sequence disclosed available in the public database. Differences between the determined cDNA sequence, and that disclosed in the public database, GeneBank Accession #AF211189, are candidate deleterious mutations. Following identification and characterization, oligonucleotides can be designed for the detection of specific mutants. Alternatively, the species of α_{1I} encoding mRNA of a patient can be examined with the technique of semi-quantitative PCR on total mRNA from the patient by sequentially using oligonucleotides specific for the different exons of the α_{1I} encoding gene.

Alternatively, the α_{1I} gene can be isolated from the genome of a patient and directly examined for mutations by such techniques as restriction mapping or sequencing.

To determine whether such mutations are responsible for the diseased phenotype, experiments can be designed in which the defective gene carrying the identified mutation is introduced into a cell system expressing a complement of components sufficient for the production of functional neuronal voltage-gated calcium channels. The ability of the mutant α_{1I} to function in the calcium can be assessed using conventional techniques, such as the ones described above.

Another aspect of the present invention includes the complementation of a defective mutant gene (e.g., identified as described above) in an affected individual by the introduction of a genetic construct carrying DNA encoding functional α_{1I}. The introduction of such a complementary copy of the α_{1I} coding sequence can be accomplished through the use of any of the conventional techniques which are known to be effective.

Among the several aspects of the invention, therefore, is provided a method for the treatment, inhibition, or prevention of an α_{1I} calcium channel mediated disorder in a subject in need thereof. The method comprises administering to the subject an amount of an inhibitor of T-type Ca²⁺ channel activity or a pharmaceutically acceptable salt or prodrug thereof effective to treat, inhibit or prevent unwanted pain perception in the subject. In other aspects, the invention relates to expression vectors which encode neuronal-specific subunits, as well as cells containing such vectors.

An additional aspect of the invention is drawn to methods for producing transgenic non-human animals, wherein the animal expresses a reduced level of calcium channel α_{1I} subunit relative to a corresponding wild-type animal, comprising: a) providing: i) an embryonic stem cell comprising wild-type all-subunit genes; ii) a blastocyst of a non-human animal; iii) a pseudopregnant non-human animal; and iv) an oligonucleotide sequence comprising at least a portion of a non-human nucleotide sequence homologous to a nucleic acid sequence selected from the group consisting of (1) SEQ ID NO:18, complements thereof and variants thereof, and (2) SEQ ID NO:20, complements thereof and variants thereof; b) introducing the oligonucleotide sequence into the embryonic stem cell under conditions such that the oligonucleotide sequence is homologously recombined into at least one of the wild-type α_{1I} subunit genes in the genome of the embryonic stem cell to produce a treated embryonic stem cell; c) injecting the treated embryonic stem cell into the blastocyst to produce an injected blastocyst; d) introducing the injected blastocyst into the pseudopregnant non-human animal; and e) permitting the pseudopregnant animal to deliver progeny comprising the homologously recombined oligonucleotide, wherein the progeny express a reduced level of calcium channel α_{1I} subunit relative to a corresponding wild-type animal.

In one preferred embodiment, the transgenic non-human animal is selected from the order Rodentia. In a more preferred embodiment, the transgenic non-human animal is a mouse.

The invention also provides methods for producing transgenic non-human animals, wherein the animal expresses reduced activity of calcium channel α_{1I} subunit relative to a corresponding wild-type animal, comprising: a) providing: i) an embryonic stem cell comprising wild-type calcium channel α_{1I} subunit genes; ii) a blastocyst of a non-human animal; iii) a pseudopregnant non-human animal; and iv) an oligonucleotide sequence comprising at least a portion of a non-human nucleotide sequence homologous to a nucleic acid sequence selected from the group consisting of (1) SEQ ID NO:18, complements thereof and variants thereof, and (2) SEQ ID NO.:20, complement thereof and variants thereof, complements thereof and variants thereof, wherein at least portion of the nucleotide sequence comprises one or more mutations selected from the group consisting of deletion, insertion and point mutation(s); b) introducing the oligonucleotide sequence into the embryonic stem cell under conditions such that the oligonucleotide sequence is homologously recombined into at least one of the wild-type calcium channel α_{1I}-subunit genes in the genome of the embryonic stem cell to produce a treated embryonic stem cell; c) injecting the treated embryonic stem cell into the blastocyst to produce an injected blastocyst; d) introducing the injected blastocyst into the pseudopregnant non-human animal; and e) permitting the pseudopregnant animal to deliver progeny comprising the homologously recombined oligonucleotide, wherein the progeny express reduced activity of α_{1I} non-human animal is selected from the order Rodentia. In a more preferred embodiment, the transgenic non-human animal is a mouse.

In accordance with the above, the invention provides a method for identifying a therapeutic compound, comprising: a) providing: i) a transgenic non-human animal produced by the method described in either of the previous two paragraphs; and ii) a composition comprising the compound; and b) administering the compound to the transgenic non-human animal to produce a treated animal.

While it is not intended that the transgenic animal be limited to a particular type, in one preferred embodiment, the transgenic animal has an α_{1I} mediated disorder. In another preferred embodiment, the transgenic animal has a neurological disease selected from the group consisting of epilepsy, stroke, brain trauma, Alzheimer's disease, multiinfarct dementia, amyotrophic lateral sclerosis, convulsions, seizures, Huntington's disease, and amnesia. In yet another preferred embodiment, the transgenic animal has a cardiovascular disease selected from the group consisting of cardiac arrhythmia, angina pectoris, hypoxic damage to the cardiovascular system, ischemic damage to the cardiovascular system, myocardial infarction, and congestive heart failure. In a further preferred embodiment, the transgenic animal has Lambert-Eton myasthenic syndrome.

The invention further comprises pharmaceutical compositions to carry out the above described methods.

Other aspects of the present invention are presented in the accompanying claims and in the following description and drawings. These aspects are presented under separate section headings. However, it is to be understood that the teachings under each section are not necessarily limited to that particular section heading.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1. Depicts the results of a Western blot for α_{1I} peptide-antibody (#732) complex in an α_{1I} stable cell line.

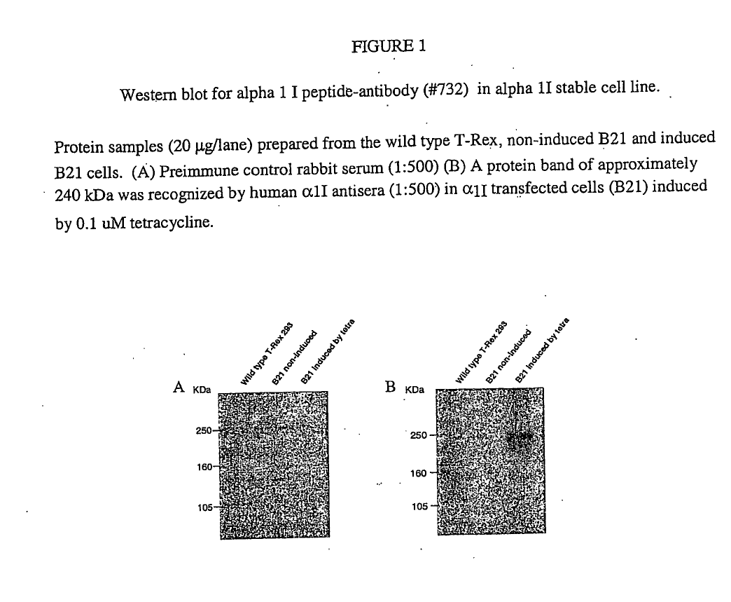

FIG. 2. Time course studies of calcium influx through the α_{1I} channel in B21 cells.

FIG. 3. Calcium influx in wild type cells and B21 cells

FIG. 4. Electrophysiological characterization of the α_{1I} channel activation and inactivation properties when expressed the T-Rex cell line.

FIG. 5. Depicts the alignment of the amino acid sequence of SEQ ID NO:19 and SEQ ID NO:21.

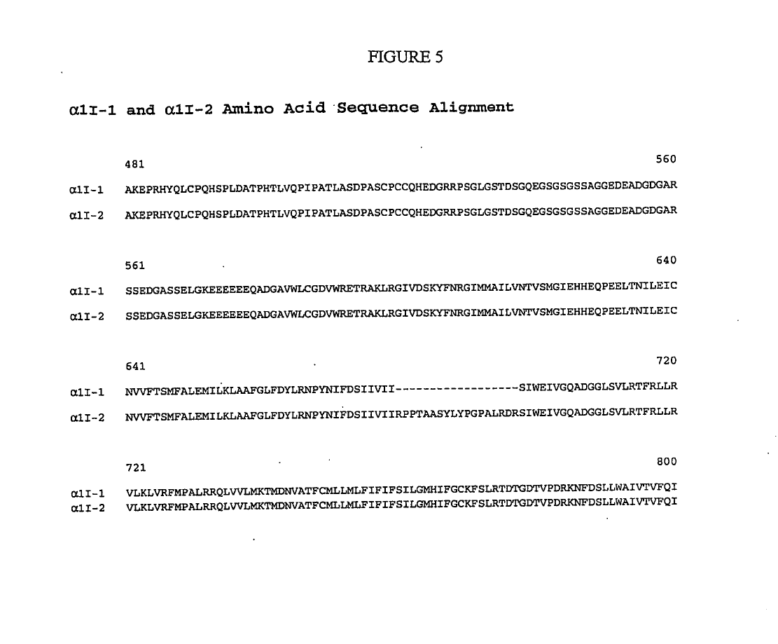

FIG. 6. Depicts the alignment of the nucleotide sequences encompassed by SEQ ID NO:18 and SEQ ID NO:20.

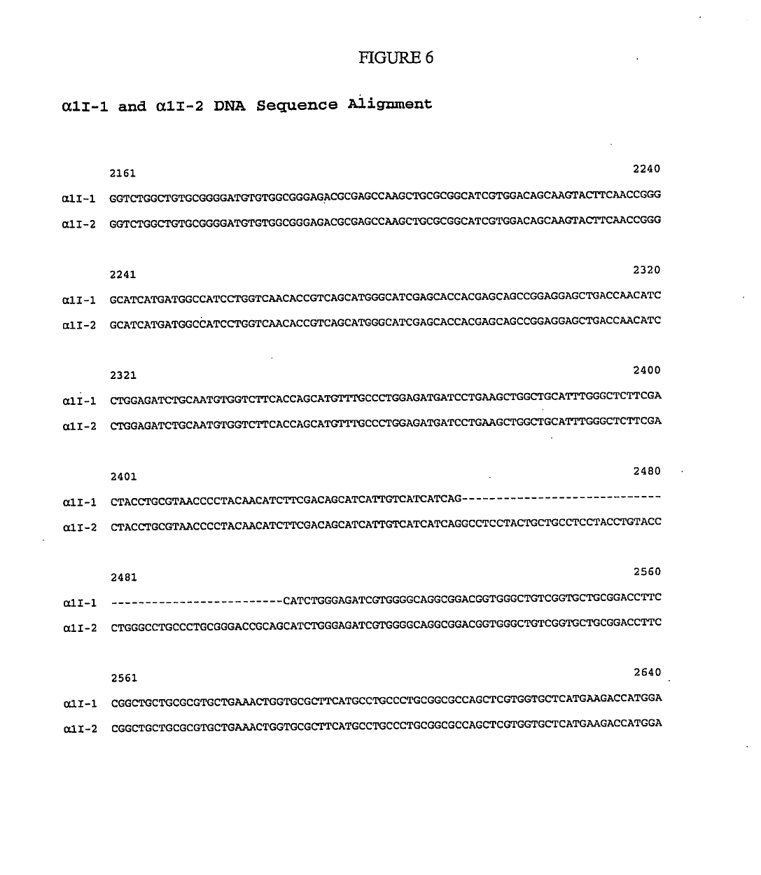

### DETAILED DESCRIPTION OF THE INVENTION

Before the present proteins, nucleotide sequences, and methods are described, it is to be understood that the present invention is not limited to the particular methodologies, protocols, cell lines, vectors, and reagents described, as these may vary. It is also understood that the terminology used herein is for the purpose of describing particular embodiments only, and is not to limit the scope of the present invention.

The singular forms “a,” “an,” and “the” include plural reference unless the context clearly dictates otherwise.

All technical and scientific terms used herein have the same meanings as commonly understood by one of ordinary skill in the art to which this invention pertains. The practice of the present invention will employ, unless otherwise indicated, conventional techniques of protein chemistry and biochemistry, molecular biology, microbiology and recombinant DNA technology, which are within the skill of the art. Such techniques are explained fully in the literature.

Although any machines, materials, and methods similar or equivalent to those described herein can be used to practice or test the present invention, the preferred machines, materials, and methods are now described. All patents, patent applications, and publications mentioned herein, whether supra or infra, are each incorporated by reference in its entirety. It is noted that the discussion detailed herein pertaining to the α_{1I-1} isoform, SEQ ID NOS:18 and 19 applies equally to the α_{1I-2} isoform, SEQ ID NOS.20 and 21, disclosed herein. For example, just as the human T-type calcium channel α_{1I-1} subunit isoform encoding nucleic acid is “isolated”, so is the human T-type calcium channel α_{1I-2} subunit isoform encoding nucleic acid molecule etc.

### GLOSSARY

In the following description and claims use will be made, at times, with a variety of terms, and the meaning of such terms as they should be construed in accordance with the invention is as follows:

In the following commentary, reference to “nucleotide sequence of the present invention” and “amino acid sequence of the present invention” refer respectively to any one or more nucleotide sequences presented or discussed herein and to any one or more of the amino acid sequences presented or discussed herein. Also, and as used herein, “amino acid” refers to peptide or protein sequence and may refer to portions thereof. In addition, the term “amino acid sequence of the present invention” is synonymous with the phrase “polypeptide of the present invention”. Also the term “nucleotide sequence of the present invention” is synonymous with the phrase “poly-nucleotide sequence of the present invention”.

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention protein.

In defining nucleic acid sequences, all subject nucleic acid sequences capable of encoding “substantially similar amino acid sequences” are considered substantially similar or are considered as comprising substantially identical sequences of nucleotides to the reference nucleic acid sequence, i.e., α_{1I-1} isoform encoding sequence—SEQ ID NO:18.

Nucleotide sequence “similarity” is a measure of the degree to which two polynucleotide sequences have identical nucleotide bases at corresponding positions in their sequence when optimally aligned (with appropriate nucleotide insertions or deletions). Sequence similarity or percent similarity can be determined, for example, by comparing sequence information using sequence analysis software such as the GAP computer program, version 6.0, available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math. 2:482, 1981).

The phrases “percent identity” and “% identity” refers to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. “Identity” can be readily calculated by known methods. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman (1981) Advances in Appl. Math. 2:482-489, for peptide analysis. Programs for determining nucleotide sequence identity are available in the Wisconsin Sequence Analysis Package, Version 8 (Genetics Computer Group, Madison, Wis.) for example, the BLAST, BESTFIT, FASTA, and GAP programs, which also rely on the Smith and Waterman algorithm. These programs are readily utilized with the default parameters recommended by the manufacturer and described in the Wisconsin Sequence Analysis Package referred to above. Other programs for calculating identity or similarity between sequences are known in the art.

The well known Smith Waterman algorithm may also be used to determine identity.

Parameters for polypeptide sequence comparison include the following:

1) Algorithm: Needleman and Wunsch, J. Mol. Biol. 48: 443-453 (1970)

Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following:

1) Algorithm: Needleman and Wunsch, J. Mol. Biol. 48: 443-453 (1970)

Comparison matrix: matches=+10, mismatch=0

Gap Penalty: 50

Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 50, 60, 70, 80, 85, 90, 95, 97 or 100% identity to the reference sequence of SEQ ID NO:18, wherein the polynucleotide sequence may be identical to the reference sequence of SEQ ID NO:18 or may include up to a certain integer number of nucleotide alterations as compared to the reference sequence, wherein the alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein the number of nucleotide alterations is determined by multiplying the total number of nucleotides in SEQ ID NO:18 by the integer defining the percent identity divided by 100 and then subtracting that product from the total number of nucleotides in SEQ ID NO:18, or:

Nn Xn−(Xn Y),

wherein Nn is the number of nucleotide alterations, Xn is the total number of nucleotides in SEQ ID NO:18, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xn and Y is rounded down to the nearest integer prior to subtracting it from Xn. Alterations of a polynucleotide sequence encoding the polypeptide of SEQ ID NO:19 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 50, 60, 70, 80, 85, 90, 95, 97 or 100% identity to a polypeptide reference sequence of SEQ ID NO:19, wherein the polypeptide sequence may be identical to the reference sequence of SEQ ID NO:19 or may include up to a certain integer number of amino acid alterations as compared to the reference sequence, wherein the alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein the number of amino acid alterations is determined by multiplying the total number of amino acids in SEQ ID NO:19 by the integer defining the percent identity divided by 100 and then subtracting that product from the total number of amino acids in SEQ ID NO:19, or:

Na=Xa−(Xa Y),

herein Na is the number of amino acid alterations, Xa is the total number of amino acids in SEQ ID NO:19, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xa and Y is rounded down to the nearest integer prior to subtracting it from Xa.

For example, a designated amino acid percent identity of 70% refers to sequences or subsequences that have at least about 70% amino acid identity when aligned for maximum correspondence over a comparison window as measured using one of the sequence comparison algorithms disclosed herein and well known to a skilled artisan or by manual alignment and visual inspection. It is recognized, however, that proteins (and DNA or mRNA encoding such proteins) containing less than the above-described level of homology arising as variants or that are modified by conservative amino acid substitutions (or substitution of degenerate codons) are contemplated to be within the scope of the present invention.

“Optimal alignment”—is defined as an alignment giving the highest percent identity score. Such alignment can be performed using a variety of commercially available sequence analysis programs, such as the local alignment program LALIGN using a ktup of 1, default parameters and the default PAM. A preferred alignment is the one performed using the CLUSTAL-W program from MacVector™, operated with an open gap penalty of 10.0, an extended gap penalty of 0.1, and a BLOSUM similarity matrix. If a gap needs to be inserted into a first sequence to optimally align it with a second sequence, the percent identity is calculated using only the residues that are paired with a corresponding amino acid residue (i.e., the calculation does not consider residues in the second sequences that are in the “gap” of the first sequence). In case of alignments of known gene sequences with that of the new variant, the optimal alignment invariably included aligning the identical parts of both sequences together, then keeping apart and unaligned the sections of the sequences that differ one from the other.

“Having at least 90% identity”—with respect to two amino acid or nucleic acid sequence sequences, refers to the percentage of residues that are identical in the two sequences when the sequences are optimally aligned. Thus, 90% amino acid sequence identity means that 90% of the amino acids in two or more optimally aligned polypeptide sequences are identical, however this definition explicitly excludes sequences which are 100% identical with the original sequence from which the variant of the invention was varied.

“Homologues of variants”—amino acid sequences of variants in which one or more amino acids has been added, deleted or replaced. The addition, deletion or replacement should be in regions or adjacent to regions where the α_{1I} and α_{1I-2} variants differs from the original α_{1I} sequence.

“Conservative substitution”—refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. [Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gln.

T-type-selective inhibitors are defined herein as compounds which inhibit the Ca²⁺ VG current but which do not effect the non-voltage gated current. Such compounds are preferably screened using the assays described herein.

As used herein “human T-type calcium channel α_{1I} isoform activity” or human T-type calcium channel α_{1I-1} or α_{1I-2} subunit activity” or grammatical equivalents thereof refers to an ability of a molecule to modulate voltage regulated, i.e., T-type calcium influx. A molecule which inhibits human T-type calcium channel α_{1I} subunit activity (an antagonist) is one which inhibits voltage regulated calcium influx via this calcium channel and a molecule which increases human T-type calcium channel α_{1I} subunit activity (an agonist) is one which increases voltage regulated calcium influx via this calcium channel. Changes in human T-type calcium channel α_{1I} isoform subunit activity can be measured by changes in voltage regulated calcium influx by in vitro assays such as those disclosed herein, including patch-clamp assays and assays employing calcium sensitive fluorescent compounds such as fura-2.

“Agonist” refers to a molecule which, when bound to α_{1I-1} or α_{1I-2}, or is within proximity of α_{1I-1} or α_{1I-2}, modulates the activity α_{1I} or α_{1I-2} by increasing or prolonging the duration of the effect of α_{1I-1} or α_{1I-2}. Agonists can include proteins, nucleic acid molecules, carbohydrates, organic compounds, inorganic compounds, or any other molecules which modulate the effect of α_{1I-1} or α_{1I-2}.

“Antagonist” refers to a molecule which, when bound to α_{1I-1} or α_{1I-2} or within close proximity, decreases the amount or the duration of the biological or immunological activity of α_{1I-1} or α_{1I-2}. Antagonists may include proteins, nucleic acid molecules, carbohydrates, antibodies, organic compounds, inorganic compounds, or any other molecules which exert an effect on α_{1I-1} or α_{1I}-2 activity.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

“Antibody”—refers to IgG, IgM, IgD, IgA, and IgG antibody. The definition includes polyclonal antibodies or monoclonal antibodies. This term refers to whole antibodies or fragments of the antibodies comprising the antigen-binding domain of the anti-variant product antibodies, e.g. antibodies without the Fc portion, single chain antibodies, fragments consisting of essentially only the variable, antigen-binding domain of the antibody, etc. As such, “Antibody” can be an intact molecule or fragments thereof, such as Fab, F(ab)₂, and Fv fragments, which are capable of binding an epitopic determinant. The antibody can be polyclonal, monoclonal, or recombinantly produced.

“Treating a disease”—refers to administering a therapeutic substance effective to ameliorate symptoms associated with a disease, to lessen the severity or cure the disease, or to prevent the disease from occurring.

“Detection”—refers to a method of detection of a disease, disorder, pathological or normal condition. This term may refer to detection of a predisposition to a disease as well as for establishing the prognosis of the patient by determining the severity of the disease.

“Original α_{1I} sequence” or “reference sequence” is used interchangeably to refer to the amino acid or nucleic acid sequence from which the α_{1I} variants(s)/isoforms of the invention have been varied as a result of alternative slicing. The original sequence is the sequence of the human T-type calcium channel and the sequence is published as GenBank Accession #AF211189.

“Amplification” relates to the production of additional copies of a nucleic acid sequence. Amplification can be carried out using polymerase chain reaction (PCR) technologies or other methods well known in the art.

The term “antisense” or “antisense oligonucleotides” refers to short synthetic nucleotide sequences formulated to be complementary to a portion of a specific gene or mRNA. They function by hybridizing to complementary sequences, resulting in the selective arrest of expression of the complementary gene or mRNA. In addition to the use of antisense oligonucleotides as therapeutic agents due to their ability to block expression of a specific target protein, they also provide a useful tool for exploring regulation of the expression of a gene of interest i and in tissue culture (see Rothenberg, M., et al., Natl. Cancer Inst., 81:1539-1544, 1989).

The term “expression” as used herein intends both transcriptional and translational processes, i.e., the production of messenger RNA and/or the production of protein therefrom.

The term “modulate” refers to a change in the activity of the variants detailed herein. For example, modulation may cause an increase or a decrease in protein activity, binding characteristics, or any other biological, functional, or immunological properties of α_{1I-1} and/or α_{1I-2}. The ability to modulate the activity of α_{1I-1} or α_{1I-2} can be exploited in assays to screen for organic, inorganic, or biological compounds which affect the above properties of α_{1I-1} and/or α_{1I-2}.

A “reporter gene” is a gene that, upon expression, confers a phenotype on a cell expressing the reporter gene, such that the cell can be identified under appropriate conditions. For example, the reporter gene may produce a polypeptide product that can be easily detected or measured in a routine assay. Suitable reporter genes known in the art which confer this characteristic include those that encode chloramphenicol acetyl transferase (CAT activity), β-galactosidase, luciferase, alkaline phosphatase, human growth hormone, fluorescent proteins, such as green fluorescent protein (GFP), and others. Indeed, any gene that encodes a protein or enzyme that can readily be measured, for example, by an immunoassay such as an enzyme-linked immunosorbent assay (ELISA) or by the enzymatic conversion of a substrate into a detectable product, and that is substantially not expressed in the host cells (specific expression with no background) can be used as a reporter gene to test for promoter activity. Other reporter genes for use herein include genes that allow selection of cells based on their ability to thrive in the presence or absence of a chemical or other agent that inhibits an essential cell function. Suitable markers, therefore, include genes coding for proteins which confer drug resistance or sensitivity thereto, or change the antigenic characteristics of those cells expressing the reporter gene when the cells are grown in an appropriate selective medium. For example, reporter genes include: cytotoxic and drug resistance markers, whereby cells are selected by their ability to grow on media containing one or more of the cytotoxins or drugs; auxotrophic markers by which cells are selected by their ability to grow on defined media with or without particular nutrients or supplements; and metabolic markers by which cells are selected for, e.g., their ability to grow on defined media containing the appropriate sugar as the sole carbon source. These and other reporter genes are well known in the art.

A “change in the level of reporter gene product” is shown by comparing expression levels of the reporter gene product in a cell exposed to a candidate compound relative to the levels of reporter gene product expressed in a cell that is not exposed to the test compound and/or to a cell that is exposed to a control compound. The change in level can be determined quantitatively for example, by measurement using a spectrophotometer, spectrofluorometer, luminometer, and the like, and will generally represent a statistically significant increase or decrease in the level from background. However, such a change may also be noted without quantitative measurement simply by, e.g., visualization, such as when the reporter gene is one that confers the ability on cells to form colored colonies on chromogenic substrates.

“Subject” means mammals and non-mammals. Mammals means any member of the Mammalia class including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

The term “substantially purified,” when referring to a polypeptide, indicates that the polypeptide is present in the substantial absence of other similar biological macromolecules.

The term “transformed” refers to any known method for the insertion of foreign DNA or RNA sequences into a host prokaryotic cell. The term “transfected” refers to any known method for the insertion of foreign DNA or RNA sequences into a host eukaryotic cell. Such transformed or transfected cells include stably transformed or transfected cells in which the inserted DNA is rendered capable of replication in the host cell. They also include transiently expressing cells which express the inserted DNA or RNA for limited periods of time. The transformation or transfection procedure depends on the host cell being transformed. It can include packaging the polynucleotide in a virus as well as direct uptake of the polynucleotide, such as, for example, lipofection or microinjection. Transformation and transfection can result in incorporation of the inserted DNA into the genome of the host cell or the maintenance of the inserted DNA within the host cell in plasmid form. Methods of transformation are well known in the art and include, but are not limited to, viral infection, electroporation, lipofection, and calcium phosphate mediated direct uptake.

“Treating” or “treatment” of a disease state includes: 1) preventing the disease state, i.e. causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) or relieving the disease state, i.e., causing temporary or permanent regression of the disease state or its clinical symptoms.

A “variant” of α_{1I-1} and/or α_{1I-2} polypeptides refers to an amino acid sequence that is altered by one or more amino acid residues. The variant may have “conservative” changes, wherein a substituted amino acid has similar structural or chemical properties (e.g., replacement of leucine with isoleucine.) More rarely, a variant may have “nonconservative” changes (e.g., replacement of glycine with tryptophan.) Analogous minor variations may also include amino acid deletion or insertions, or both. Guidance in determining which amino acid variations may be substituted, inserted, or deleted without abolishing biological function may be found using programs well known in the art, for example, LASERGENE software (DNASTAR).

### The Invention

The present invention is based on the discovery of two novel human T-type calcium channel α_{1I} subunit variants (α_{1I-1} and α_{1I-2}), the polynucleotides encoding each of (α_{1I-1} and α_{1I-2}, as well as therapeutics relating thereto. The invention also embraces isolated functionally equivalent variants, useful analogs and fragments of the foregoing nucleic acid molecules and polypeptides; complements of the foregoing nucleic acid molecules; and molecules which selectively bind the foregoing nucleic acid molecules, and the use of the above for screening compounds useful in the treatment or prevention of a dysfunctional calcium channel, e.g., dysfunctional T-type voltage regulated calcium influx mediated by a (α_{1I})calcium subunit.

The novel variants of the invention may have the same physiological activity as the reference α_{1I} from which they are varied (although perhaps at a different level) considering that they each share a considerable degree of primary sequence identity with the reference sequence. However, the novel isoforms may differ from the reference sequence by their agonist-dependent or agonist independent activity. In addition, either one or both of the novel isoforms of the invention may differ from the reference sequence in their affinity to its native ligand/binding partner (calcium) or affinity to various agonist and antagonists coupling efficiency to G-protein which they can activate, or alternatively may have no activity at all and this may lead to various diseases or pathological conditions.

Both in the case the α_{1I} subunit isoform of the invention has the same activity as the reference sequence as well as the case it has the opposite activity from that of the reference sequence, it may differ from the reference sequence in its stability, its clearance rate, its rate of degradation, its tissue and cellular distribution, its ligand specification, its temporal expression pattern, its pattern and mechanism of up and down regulation and in other biological properties not necessarily connected to activity.

In addition to therapeutic purposes, the novel α_{1I} subunit isoforms may be very useful for detection purposes, i.e. its presence or level may be indicative of a disease, disorder, pathological or normal condition involving an α_{1I} isoform. Representative diseases include disorders of neurologic or psychiatric origin such as, for example, neurodegenerative disease, epilepsy, anxiety, schizophrenia, manic depression, depression, delirium, the treatment of cancer. Alternatively, the ratio between the level of either or both of the α_{1I} isoforms and the level of the reference peptide from which it has been varied, or the level of one α_{1I} isoform to the other may be indicative to such a disease, disorder, pathological or normal condition, for example, any one of the diseases specified above.

For representative purposes, it is possible to establish differential expression of the novel isoforms of the invention in various tissues as compared to the reference α_{1I} subunit sequence. The α_{1I} isoforms may be expressed mainly in one tissue, while the reference α_{1I} subunit sequence from which it has been varied may be expressed mainly in another tissue. Understanding of the distribution of the novel isoforms in various tissues may be helpful, for example, in understanding the physiological function of the genes as well as helping in targeting pharmaceuticals or developing pharmaceuticals. In addition, deviation from normal distribution may be indicative of any of the above diseases.

The study of the α_{1I} isoforms may also be helpful to distinguish various stages in the life cycles of the cells which may also be helpful for development of pharmaceuticals for various pathological conditions in which cell cycles is abnormal. Such abnormal can lead either to various developmental problems concerning the nervous system or alternatively can lead to various types of cancer, or to diseases involving the nervous system, both neuronal and psychiatric origin or any one of the diseases specified above.

The detection may be by determination of the presence or the level of expression of the α_{1I} isoforms within a specific cell population, and comparing said presence or level between various cell types in a tissue, between different tissues and between individuals.

The α_{1I} isoform molecules of the present invention were isolated as detailed in Example 1.

### I. Nucleic Acid Molecules; Vectors; and Recombinant Host Cells of the Invention.

(a) In a first aspect, the invention provides isolated nucleic acid molecules that encode a functional auxiliary subunit of a human T-type voltage-gated calcium channel. Each of the novel isoform sequences codes for a novel, naturally occurring, variant of the native and known calcium channel α_{1I} subunit, the sequence of which is disclosed in GenBank accession #AF211189. It should be emphasized that the novel isoform sequences are naturally occurring sequences resulting from either the alternative splicing of the native α_{1I} subunit gene and/or as a single nucleotide polymorphism of the reverence gene and not merely truncated, or fragmented forms of the gene. Specifically, the invention discloses nucleic acid molecules encoding novel human isoforms of the T-type calcium channel, referred to herein as the human T-type calcium channel α_{1I-1} subunit (α_{1I-1} isoform) and the human T-type calcium channel α_{1I-2} subunit (α_{1I}-2 isoform).

As used herein “human T-type calcium channel α_{1I} subunit encoding nucleic acid molecule” or “variant encoding nucleic acids” “isoform nucleic acid molecules” refers not only to an isolated nucleic acid molecule which codes for a human T-type calcium channel α_{1I-1} subunit isoform comprising the nucleotide sequence shown in SEQ ID NO:18 in the case of the α_{1I-1} or SEQ ID NO:20 in the case of α_{1I-2}, but also nucleotide sequences having at least 90% identity to said sequences (SEQ ID NOS:18 or 20) and fragments of the above sequences, which encode a biologically active T-type calcium channel subunit polypeptide isoform. In this context, “biologically active,” refers to a protein having structural, regulatory, or biochemical functions attending a naturally occurring molecule or an isoform thereof. Likewise, “immunologically active” refers to the capability of the natural, recombinant, or synthetic α_{1I} subunit isoform, e.g., SEQ ID NO:19, or of any oligopeptide thereof, to induce a specific immune response in appropriate animals or cells and to bind with specific antibodies.

As well, the term “human T-type calcium channel α_{1I} subunit nucleic acid molecules” or grammatically equivalent terms thereof also includes those nucleic acid molecules which code for a human T-type calcium channel α_{1I} subunit polypeptide isoform comprising the amino acid sequences as set forth in one of SEQ ID NOS:19 and 21. More, the nucleic acid molecules comprising nucleotide sequences which differ from the sequence of SEQ ID NOS:18 or 20 in codon sequence due to the degeneracy of the genetic code are also within the scope of the invention. The nucleic acid molecules of the invention also include biologically active fragments of the foregoing nucleic acid molecules, provided that the fragment encodes the amino acid sequence of SEQ ID NOS:19 or 21 or biologically active fragments thereof, meaning, for example, those fragments which may bind an antibody that also recognizes one of SEQ ID NO:19 or 21. The term “isolated”, as used herein in reference to a nucleic acid molecule, means a nucleic acid sequence: (i) amplified in vitro by, for example, polymerase chain reaction (PCR); (ii) synthesized by, for example, chemical synthesis; (iii) recombinantly produced by cloning; or (iv) purified, as by cleavage and electrophoretic or chromatographic separation from natural contaminants, e.g., free of other nucleic acid molecules that do not encode the subject polypeptide.

The term “nucleic acid sequence” “nucleic acid molecule” or grammatical equivalents thereof is intended for ribonucleic acid (RNA) or deoxyribonucleic acid (DNA) of genomic or synthetic origin which may be single stranded or double stranded and may represent the sense of the antisense strand, a peptide nucleic acid (PNA), or any DNA-like or RNA-like material. DNA can be either complementary DNA (cDNA) or genomic DNA.

“Peptide nucleic acid” (PNA), as used herein, refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA and RNA and stop transcript elongation, and may be pegylated to extend their lifespan in the cell. See, e.g., Nielsen, P. E. et al. (1993) Anticancer Drug Des. 8:53-63. In this context, “fragments” refer to those nucleic acid molecules which, when translated, would produce polypeptides retaining some functional characteristic, e.g., antigenicity, or structural domain, e.g., ion channel domain, calcium influx activity etc. characteristic of the full-length polypeptide.

“Complementary” and “complementarity” refer to the natural binding of polynucleotides to other polynucleotides by base pairing. For example, the sequence “5′A-C-G-T 3′” will bind to the complementary sequence “3′T-G-C-A 5′”. Complementarity between two single stranded molecules may be “partial,” such that only some of the nucleic acid molecules bind, or it may be “complete,” such that total complementarity exists between the single stranded molecules.

Also encompassed by the invention are polynucleotides that are capable of hybridizing to the claimed polynucleotide sequences, and, in particular, to those shown in one of SEQ ID NOS:18 and 20, under various conditions of stringency. (See, e.g., Wahl, G. M. and S. L. Berger (1987) Methods Enzymol. 152:399-401; Kimmel, A. R. (1987) Methods Enzymol. 152:507-511.)

“Hybridization” refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art. A hybridization complex may be formed in solution (conditions calculated by performing, e.g., Cot or Rot) or formed between one nucleic acid sequence present in solution and another nucleic acid sequence immobilized on a solid support (e.g., paper, membranes, filters, chips, pins, glass slides, or any other appropriate substrate to which cells or their nucleic acid molecules have been fixed.) For example, stringent salt concentration will ordinarily be less that about 750 mM NaCl and 75 mM trisodium citrate, preferably less than about 500 mM NaCl and 50 mM trisodium citrate, and more preferably less than about 250 mM NaCl and 25 mM trisodium citrate. Stringent temperature conditions will ordinarily include temperatures of at least about 30° C., preferably at least about 37° C., and more preferably 42° C. Varying additional parameters such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York.

As known to those of skill in the art, the stability of hybrids is reflected in the melting temperature (Tm) of the hybrids. Tm can be approximated by the formula:

81.5° C.−16.6(log 10[Na+])+0.41(% G+C)−600/1,

where 1 is the length of the hybrids in nucleotides. Tm decreases approximately 1°-1.5° C. with every 1% decrease in sequence homology. In general, the stability of a hybrid is a function of sodium ion concentration and temperature.

Typically, the hybridization reaction is performed under conditions of lower stringency, followed by washes of varying, but higher, stringency. Reference to hybridization stringency relates to such washing conditions. Low stringency hybridization can be obtained in the absence of organic solvent, e.g., formamide, while high stringency hybridization can be obtained in the presence of at least about 35% formamide, and more preferably at least about 50% formamide. There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of alleles of human T-type calcium channel α_{1I-1} subunit nucleic acid molecules of the invention. The skilled artisan also is familiar with the methodology for screening cells and libraries for expression of such molecules which then are routinely isolated, followed by isolation of the pertinent nucleic acid molecule and sequencing.

As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C. The skilled artisan will recognize how to adjust the temperature, ionic strength, etc. as necessary to accommodate factors such as probe length and the like. (See Sambrook et al., Molecular Cloning: A Laboratory Manual, New York: Cold Spring Harbor Press, 1989).

“High stringency conditions”, as defined herein, may be identified by those that: (1) employ low ionic strength and high temperature for washing, for example 0.15 M sodium chloride/0.15 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 Jig/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The phrase “low stringency hybridization” refers to conditions equivalent to hybridization in 10% formamide, 5× Denhart's solution, 6×SSPE, 0.2% SDS at 42° C., followed by washing in 1×SSPE, 0.2% SDS, at 50° C.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash step will ordinarily include temperature of at least about 25° C., more preferably of at least about 42° C., and most preferably of at least about 68° C.

More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15 M sodium chloride/0.015 M sodium citrate, pH7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1. SDS at temperatures up to 65° C.

In practice, the term “substantially the same sequence” means that DNA or RNA encoding two proteins hybridize under moderately stringent conditions and encode proteins that have the same sequence of amino acids or have changes in sequence that do not alter their structure or function.

Thus, an aspect of the invention embraces those nucleic acid molecules comprising coding sequences which are substantially the same as those set forth in SEQ ID NO:18 and which hybridize to a nucleic acid molecule comprising SEQ ID NO:18 or 20 under moderately stringent conditions.

A “coding sequence” is a polynucleotide sequence that is transcribed into mRNA and translated into a polypeptide. The boundaries of the coding sequence are determined by a translation start codon at the 5′-terminus and a translation stop codon at the 3′-terminus. A coding sequence can include, but is not limited to, mRNA, cDNA, synthetic DNA, and recombinant polynucleotide sequences. Also included is genomic DNA where the coding sequence is interrupted by introns.

Preferred nucleic acid molecules encoding the invention polypeptide(s) hybridize under moderately stringent, preferably high stringency, conditions to substantially the entire sequence, or substantial portions (i.e., typically at least 15-30 nucleotides) of the nucleic acid sequence set forth in SEQ ID NOS:18 or 20.

In screening for human T-type calcium channel α_{1I-1} subunit nucleic acid molecules, a Southern blot may be performed using the foregoing stringent conditions, together with a radioactive probe. After washing the membrane to which the DNA is finally transferred, the membrane can be placed against X-ray film to detect the radioactive signal.

The human T-type calcium channel α_{1I-1} subunit nucleic acid molecule of the invention also include degenerate nucleic acid molecules which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating human T-type calcium channel α_{1I-1} subunit polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acid molecules that differ from the biologically isolated nucleic acid molecules in codon sequence due to the degeneracy of the genetic code.

The present invention also encompasses nucleic acid molecules which differ from the nucleic acid molecule shown in SEQ ID NO:18, but which have the same phenotype. “Phenotypically similar nucleic acid molecules” are also referred to as “functionally equivalent” nucleic acid molecules.

As used herein, the phrase “functionally equivalent nucleic acid molecules” “altered nucleic acid molecules” or grammatical equivalents thereof encompasses nucleic acid molecules characterized by slight and non-consequential sequence variations that will function in substantially the same manner to produce the same protein product(s) as the nucleic acid molecules disclosed herein. These changes include those recognized by those of skill in the art as those that do not substantially alter the tertiary structure of the protein. Thus “altered” nucleic acid sequences encoding an α_{1I} subunit isoform, as described herein, include those sequences with deletions, insertions, or substitutions of different nucleotides, resulting in a polynucleotide encoding the same α_{1I-1} subunit isoform of SEQ ID NO:19 or a polypeptide with at least one functional characteristic of α_{1I-1} subunit isoform from which it is derived.

Also provided are fragments of SEQ ID NO:18. The fragments will find use as probes in Southern blot assays to identify such nucleic acid molecules, or can be used in amplification assays such as those employing PCR. For example, smaller fragments are those comprising 12, 13, 14, 15, 16, 17, 18, 20, 22, 25, 30, 40, 50, or 75 nucleotides, and every integer therebetween and are useful e.g. as primers for nucleic acid amplification procedures—use for PCR etc. Likewise, as well known to one skilled in the art, larger probes comprising 200, 250, 300, 400 or more nucleotides are preferred for certain uses including Southern blots. Fragments of the invention also can be used to produce fusion proteins for generating antibodies, use in immunoassays, or as “immunologically active fragment” thereof. “Immunologically active fragment(s)” of “immunogens” refer to those fragments which are capable of eliciting an immune response to produce antibodies immunospecific for the novel isoforms/protein(s) of the invention.

A representative example includes those fragments that are capable of raising, for example, α_{1I-1} subunit specific antibodies in a target immune system (e.g., murine or rabbit) or of competing with native α_{1I} subunit for binding to α_{1I-1} subunit-specific antibodies, and is thus useful in immunoassays for the presence of α_{1I-1} subunit peptide in a biological sample. Such immunologically active fragments typically have a minimum size of 8 to 11 consecutive amino acids. As well, it is preferable that the immunologically active fragment(s) be identical to a portion of the amino acid sequence of the human α_{1I-1} subunit protein disclosed herein or a portion of the amino acid sequence from which it is derived. Immunological methods for detecting and measuring the expression of α_{1I-1} subunit peptides using either specific polyclonal or monoclonal antibodies are known in the art. Exemplary techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on α_{1I-1} subunit peptides is preferred, but a competitive binding assay may be employed. These and other assays are well known in the art. (See, e.g., Hampton, R. et al. (1990) Serological Methods, a Laboratory Manual, APS Press, St Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.).

The foregoing nucleic acid fragments of the invention further include those sequences that can be used as antisense molecules to inhibit the expression of human T-type calcium channel α_{1I-1} subunit nucleic acid molecules and polypeptides, particularly for therapeutic purposes as noted below. The foregoing fragments do not necessarily produce biologically active fragments of the isoform nucleic acid molecules since these fragments do not, if translated, produce polypeptides retaining some functional characteristic, e.g., antigenicity, or structural domain, e.g., ion channel domain, calcium influx activity etc. characteristic of the full-length polypeptide.

An alternative embodiment features a human T-type calcium channel α_{1I-1} subunit isoform encoding nucleic acid, operably linked to a gene expression sequence which directs the expression of the human T-type calcium channel α_{1I-1} subunit within a eukaryotic or prokaryotic cell. The term “gene expression sequence” refers to any regulatory nucleotide sequence, such as a promoter sequence or promoter-enhancer combination, which facilitates the efficient transcription and translation of the subject α_{1I} subunit isoform nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney murine leukemia virus and other retroviruses, and the thymidine kinase promoter of herpes simplex virus. Other constitutive promoters are known to those of ordinary skill in the art. The promoters useful as gene expression sequences of the invention also include inducible promoters—these are expressed in the presence of an inducing agent. For example, the metallothionein promoter is induced to promote transcription and translation in the presence of certain metal ions. Other inducible promoters are known to those of ordinary skill in the art.

Generally, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined human T-type calcium channel α_{1I-1} subunit nucleic acid. The gene expression sequences may further include enhancer sequences or upstream activator sequences as desired.

The subject α_{1I-1} subunit isoform nucleic acid sequence and the gene expression sequence are “operably linked” when they are covalently linked in such a way as to place the transcription and/or translation of the subject α_{1I} subunit isoform coding sequence under the influence or control of the gene expression sequence. In the event it is desired that a particular α_{1I} subunit isoform sequence be translated into a functional protein, two DNA sequences are said to be operably linked if induction of a promoter in the 5′ gene expression sequence results in the transcription of the target protein, vis-à-vis the desired a I subunit isoform, e.g., human T-type calcium channel α_{1I-1} subunit and if the nature of the linkage between the two DNA sequences does not (1) result in the introduction of a frame-shift mutation, (2) interfere with the ability of the promoter region to direct the transcription of the desired α_{1I} subunit isoform sequence, or (3) interfere with the ability of the corresponding RNA transcript to be translated into a protein. Consequently, a gene expression sequence is operably linked to an α_{1I} subunit isoform nucleic acid molecule if the gene expression sequence is capable of effecting transcription of the gene product encoded by the desired isoform nucleic acid sequence such that the resulting transcript might be translated into the desired protein or polypeptide.

The α_{1I} isoform encoding nucleic acid molecules of the invention including any inhibitors of said nucleotide sequences or encoded proteins can be delivered to the eukaryotic or prokaryotic cell alone or in association with a vector. The term “vector, in its broadest sense refers to any vehicle capable of facilitating: (1) delivery of a nucleic acid molecule of the invention, to a target cell or (2) uptake of an α_{1I} isoform nucleic acid or isoform polypeptide by a target cell. Preferably, the vectors transport the subject α_{1I} subunit isoform nucleic acid or polypeptide isoform into the target cell with reduced degradation relative to the extent of degradation that would result in the absence of the vector. Optionally, a “targeting ligand” can be attached to the vector to selectively deliver the vector to a cell which expresses on its surface the cognate receptor (e.g. a receptor, an antigen recognized by an antibody) for the targeting ligand. In this manner, the vector (containing the desired α_{1I} subunit isoform nucleic acid or its encoded gene product can be selectively delivered to a specific cell.

In general, vectors useful in the practice of the invention can be divided into two classes: biological vectors and chemical/physical vectors. For example, biological vectors are more useful for delivery/uptake of the desired human T-type calcium channel α_{1I} isoform nucleic acid molecules to/by a target cell, whereas chemical/physical vectors are more useful for delivery/uptake of a human T-type calcium channel α_{1I} isoform subunit nucleic acid molecules or human T-type calcium channel α_{1I-1} subunit proteins to/by a target cell.

Exemplary biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be attached to the nucleic acid sequences of the invention. Viral vectors are a preferred type of biological vector and include, but are not limited to, nucleic acid sequences from the following viruses: retroviruses, such as Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes virus; vaccinia virus; and polio virus. One can readily employ other vectors not named but known in the art.

Preferred viral vectors are based on non-cytopathic eukaryotic viruses in which non-essential genes have been replaced with the gene of interest, e.g., α_{1I-1} subunit encoding nucleic acid molecule. Non-cytopathic viruses include retroviruses, the life cycle of which involves reverse transcription of genomic viral RNA into DNA with subsequent proviral integration into host cellular DNA. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are detailed in Kriegler, M., “Gene Transfer and Expression, A Laboratory Manual,” W. H. Freeman C. O., New York (1990) and Murry, E. J. Ed. “Methods in Molecular Biology,” vol. 7, Humana Press, Inc., Clifton, N.J. (1991).

Another preferred virus for certain applications is the adeno-associated virus, a double-stranded DNA virus. The adeno-associated virus can be engineered to be replication-deficient and is capable of infecting a wide range of cell types and species. Other advantages include features such as heat and lipid solvent stability; high transduction frequencies in cells of diverse lineages, including hemopoietic cells; and lack of superinfection inhibition thus allowing multiple series of transductions. As well, it has been reported that the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, thus implying that the adeno-associated virus genomic integration is a relatively stable event. The adeno-associated virus can also function in an extrachromosomal fashion.

Expression vectors comprising all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding the desired all polypeptide isoform or fragment or variant thereof. That heterologous DNA (RNA) is placed under operable control of transcriptional elements to permit the expression of the heterologous DNA in the host cell.

Conspicuously comprehended are is a non-human cell line a recombinant non-human cell line which has been engineered to express a heterologous protein, the cell line comprising at least one host cell transformed or transfected with a heterologous nucleic acid molecule of the invention, e.g., SEQ ID NOS:18 or 20 under conditions favoring expression of a polypeptide of the invention.

Systems suitable for mRNA expression in mammalian cells include pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. In addition, the pCEP4 vector (Invitrogen), which contains an Epstein Barr virus (EBV) origin of replication, facilitating the maintenance of plasmid as a multicopy extrachromosomal element is very suitable for expression in primate or canine cell lines. The pEF-BOS plasmid containing the promoter of polypeptide Elongation Factor 1α, which stimulates efficiently transcription in vitro is also suitable for mRNA expression. See., e.g., Mishizuma and Nagata (Nuc. Acids Res. 18:5322, 1990), wherein the plasmid is described. See also, by and its use in transfection experiments is disclosed by, for example, Demoulin (Mol. Cell. Biol. 16:4710-4716, 1996), which details its use in transfection experiments. Other preferred expression vector include an adenovirus, described by Stratford-Perricaudet, which is defective for E1 and E3 proteins (J. Clin. Invest. 90:626-630, 1992).

In addition to biological vectors, one skilled in the art may also make use of chemical/physical vector to deliver the desired α_{1I-1} subunit isoform encoding nucleic acid molecule or polypeptide(s) to a target cell and facilitate uptake thereby. The term “Chemical/physical vector” refers to a natural or synthetic molecule, other than those derived from bacteriological or viral sources, capable of delivering an isolated α_{1I} subunit isoform nucleic acid or polypeptide to a cell.

An exemplary vector that falls within this class is a colloidal dispersion system. Colloidal dispersion systems include lipid-based systems including oil-in-water emulsions, micelles, mixed micelles, and liposomes. Preferably, the colloidal system is a liposome, which are artificial membrane vesicles which are useful as a delivery vector in vivo or in vitro. Fraley, et al., Trends Biochem. Sci., v. 6, p. 77, 1981 have shown that large unilamellar vesicles (LUV), which range in size from 0.2-4.0μ can encapsulate large macromolecules. RNA, DNA, and intact virions can be encapsulated within the aqueous interior and deliver biologically active forms of said cargo to cells. However, for a liposome to be an efficient nucleic acid transfer vector, one or more of the following characteristics should be present: (1) encapsulation of the nucleic acid of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information. Reportedly, liposomes may be targeted to a particular tissue by coupling the liposome to a specific ligand such as a monoclonal antibody, sugar, glycolipid, or protein. Ligands useful for targeting a liposome to a particular cell depends on the particular cell or tissue type. As well, in cases where the vector encapsulates a nucleic acid, the vector may be coupled to a nuclear targeting peptide, which will facilitate delivery of the desired α_{1I} subunit isoform nucleic acid to the nucleus of the host cell. Liposomes are commercially available from Gibco BRL, for example, as LIPOFECTIN™ and LIPOFECTACE™, which are formed of cationic lipids such as N-[1-(2,3 dioleyloxy)-propyl]-N,N,N-trimethylammonium chloride (DOTMA) and dimethyl dioctadecylammonium bromide (DDAB). One skilled in the art is well aware of methods for making liposomes and are described in many publications. For a review of Liposomes in general, refer to Gregoriadis, G. in Trends in Biotechnology, V. 3, p. 235-241 (1985).

Other exemplary compositions that can be used to facilitate uptake by a target cell of an α_{1I} subunit isoform nucleic acid molecules include calcium phosphate and other chemical mediators of intracellular transport, microinjection compositions, electroporation and homologous recombination compositions (e.g., for integrating the desired α_{1I} subunit isoform nucleic acid into a preselected location within a target cell chromosome).

Consequently, one skilled in the art will readily recognize that the invention also embraces the use of herein disclosed cDNA sequences in expression vectors, as well as to transfect host cells and cell lines, be these prokaryotic (e.g., E. coli), or eukaryotic (e.g., COS cells, yeast expression systems and recombinant baculovirus expression in insect cells). Especially useful are mammalian cells such as human, pig, goat, primate, etc. They may be of a wide variety of tissue types, and include primary cells and cell lines. Specifically, neuronal cells including PC 12 cells, Xenopus oocytes, bone marrow stem cells and embryonic stem cells.

Yet another aspect of the invention concerns expression kits, which enable the artisan to prepare a desired expression vector or vectors. Such expression kits include at least separate portions of the coding sequences of the herein disclosed nucleic acid molecules. Other components may be added, as desired.

(b) Peptide Nucleic Acids or “PNAs”: In yet another embodiment, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

Consequently, PNAs of the novel human T-type calcium channel α_{1I} subunit isoform encoding nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of the nucleic acid molecules of the invention can also be used in the analysis of single base pair mutations in a gene, (e.g., by PNA-directed PCR clamping); as “artificial restriction enzymes” when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of the invention can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the nucleic acid molecules of the invention can be generated which may combine the advantageous properties of PNA and DNA.

Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17):3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides of the invention can be modified with hybridization-triggered cleavage agents (See, e.g., Krol et. al. (1988) Bio-Techniques 6:958-976) or intercalating agents. (See, e.g., Zon (1988) Pharm. Res. 5:539-549). To this end, the oligonucleotides may be conjugated to another molecule, (e.g., a peptide, hybridization triggered cross-linking agent, transport agent, or hybridization-triggered cleavage agent).

### II. Substantially Pure α_{1I-1} and α_{1I-2} Polypeptides

Polypeptides—The present invention further provides substantially pure human T-type calcium channel α_{1I} isoforms designated α_{1I-1} and α_{1I-2} subunit polypeptides, preferably comprising the amino acid sequence of SEQ ID NO:19 and 21 respectively, each of which, in turn, is encoded by the novel isolated nucleic acid molecules described above —SEQ ID NO:18 and SEQ ID NO:20. “α_{1I-1} or α_{1I-2} variant(s)” collectively referred to as the “α_{1I} isoforms” or “variants”—is an amino acid sequence encoded by the α_{1I} variant nucleic acid sequences, each of which is a translation product of naturally occurring mRNA sequence obtained as a result of alternative splicing. The amino acid sequence may be a peptide, a protein, as well as peptides or proteins having chemically modified amino acids such as a glycopeptide or glycoprotein. The term also includes homologues of said sequences in which one or more amino acids has been added, deleted, substituted or chemically modified as well as fragments of this sequence having at least 90 amino acids or of being sufficient length so as to form a biologically active calcium channel. “Polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

Significantly, the novel proteins of the invention include “altered” or “variant” proteins which may contain deletions, insertions, or substitutions of amino acid residues which produce a silent change and result in a functionally equivalent or biologically equivalent isoform of α_{1I} subunit, e.g., functionally equivalent to the protein of SEQ ID NO:19 or fragments thereof. Deliberate amino acid substitutions or conservative amino acid substitutions may be made on the basis of similarity in polarity, charge, solubility, hydrophobicity, hydrophilicity, and/or the amphipathic nature of the residues, as long as the biological or immunological activity of a human T-type calcium channel subunit is retained. Altered isoforms of the invention can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the human T-type calcium channel α_{1I-1} subunit polypeptides include conservative amino acid substitutions of SEQ ID NO:19. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Thus, for example, example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

Consequently, the novel α_{1I} subunit polypeptide isoforms include any human T-type calcium channel clone having the amino acid sequence as set forth on in SEQ ID NO:19 or SEQ ID NO:21 or a biologically active fragment thereof or one having an amino acid sequence that is substantially similar to one of SEQ ID NOS:19 or 21. Importantly, while most of the foregoing discussion details the α_{1I-1} subunit (SEQ ID NO:19), it is understood that the same discussion applies equally to the α_{1I-2} subunit (SEQ ID NO:21).

As used herein, an “altered” human T-type calcium channel α_{1I} isoform is a polypeptide which contains one or more modifications to the primary amino acid sequence of a human T-type calcium channel α_{1I} subunit polypeptide isoform of the invention, e.g., SEQ ID NO:19. In this context, “altered” isoforms of the invention refer to those polypeptides that are biologically equivalent (active) to the polypeptides of SEQ ID NO:19, for example, meaning that the altered protein retains some functional characteristics of the full-length polypeptide, e.g., antigenicity, or structural domain vis-a-vis ion channel domain, its ability to regulate voltage gated calcium influx etc. Whether a particular variant protein is biologically equivalent to a target protein can be assessed based upon some physiologically measurable effect on target cells, molecules or tissues. Biological activity can also be demonstrated in a number of assays, e.g., patch clamp etc well known to one skilled in the art. For example, a biologically equivalent variant or altered α_{1I-1} subunit polypeptide retains the ability to bind ligand and/or transduce a voltage gated calcium current as does the protein of SEQ ID NO:19. Other functionally equivalent variants will be known to one of ordinary skill in the art, as will methods for preparing such variants.

The aforementioned modifications which result in an altered human T-type calcium channel α_{1I} isoform can be made to the novel variant proteins of the invention for a variety of reasons, including 1) to reduce or eliminate an activity of a human T-type calcium channel subunit, e.g., α_{1I-1} subunit polypeptide, such as voltage gated calcium influx; 2) to enhance a property of a human T-type calcium channel α_{1I-1} subunit polypeptide, such as protein stability in an expression system or the stability of protein-protein binding; 3) to provide a novel activity or property to a human T-type calcium channel α_{1I-1} subunit polypeptide, such as addition of an antigenic epitope/antigenic peptide or addition of a detectable moiety; or 4) to establish that an amino acid substitution does or does not affect voltage gated calcium influx. Such modifications are typically made to the nucleic acid which encodes the target human T-type calcium channel α_{1I} subunit polypeptide, e.g., SEQ ID NOS:19 or 21 and can include deletions, point mutations, truncations, amino acid substitutions and additions of amino acids or non-amino acid moieties. In the alternative, the proposed modifications can be made directly to the target polypeptide, such as by cleavage, addition of a linker molecule, addition of a detectable moiety, such as biotin, addition of a fatty acid, and the like. Modifications further embrace fusion proteins comprising all or part of the amino acid of SEQ ID NO:19, for example.

One of skill in the art is deemed to be familiar with methods for predicting the effect on protein conformation of a change in protein sequence, and can thus “design” an “altered” isoform embraced by the invention according to known methods. See, e.g., Dahiyat and Mayo, Science 278:82-87, 1997, for one such exemplary method whereby proteins can be designed de novo. The method can be applied to a known protein to vary only a portion of the polypeptide sequence. By applying the computational methods of Dahiyat and Mayo, specific variants of a cancer associated antigen polypeptide have been proposed and tested to determine whether the variant retains a desired conformation.

Preferred altered isoforms the novel α_{1I-1} subunit polypeptide which is modified specifically to alter a feature of the polypeptide unrelated to its physiological activity. For example, cysteine residues can be substituted or deleted to prevent unwanted disulfide linkages. Similarly, certain amino acids can be changed to enhance expression of the protein by eliminating proteolysis by proteases in an expression system (e.g., dibasic amino acid residues in yeast expression systems in which KEX2 protease activity is present).

Mutations of a nucleic acid which encode an altered isoform of the invention preferably preserve the amino acid reading frame of the coding sequence, and preferably do not create regions in the nucleic acid which are likely to hybridize to form secondary structures, such as hairpins or loops, which can be deleterious to expression of the variant polypeptide.

Mutations can be made by specifically selecting an amino acid substitution, or by random mutagenesis of a selected site in a nucleic acid which encodes the target polypeptide. Altered polypeptides are then expressed and tested for one or more activities to determine which mutation provides the target protein with a desired property. Additional mutations can be made to the altered isoform or the corresponding reference variant of the invention e.g., altered α_{1I-1} subunit or to the corresponding protein of SEQ ID NO:19, which are silent as to the amino acid sequence of the polypeptide, but which provide preferred codons for translation in a particular host. The preferred codons for translation of a nucleic acid in, e.g., E. coli, are known to those of ordinary skill in the art. Still other mutations can be made to the noncoding sequences of a human T-type calcium channel α_{1I-1} subunit gene or cDNA clone to enhance expression of the polypeptide.

The activity of an altered isoform of the invention can be tested by cloning the gene encoding the altered human T-type calcium channel e.g., α_{1I-1} isoform subunit polypeptide into a bacterial or mammalian expression vector, introducing the vector into an appropriate host cell, expressing the desired target polypeptide, and testing it for a functional capability characteristic of the corresponding novel isoform of the invention, e.g., the α_{1I-1} subunit polypeptide. For example, the altered α_{1I-1} subunit polypeptide isoform can be tested for ability to provide voltage regulated calcium influx. Preparation of other altered polypeptides may favor testing of other activities, as will be known to one of ordinary skill in the art. See also the method detailed in Lin et al., Neuron 18:153-166, 1997, and U.S. Pat. No. 5,429,921, to Harpold et al., entitled “Assays for agonists and antagonists of recombinant human calcium channels”. Altered α_{1I} subunit polypeptides, derived from either one of the novel isoforms disclosed herein (SEQ ID NOS:19 or 21) will find use, inter alia, in assays for identifying compounds that bind and/or regulate the calcium influx function of the corresponding α_{1I} isoform of the invention, e.g., SEQ ID NO:19. Such variants will also be useful for determining the portions of the corresponding channel α_{1I} isoform, e.g., SEQ ID NO: 9 that is required for calcium influx activity. Non-functional variants, on the other hand, will find use, for example, as antagonists of calcium channel function and as negative controls in experiments designed to test subunit activity etc.

In a preferred embodiment, the biologically active variants of the invention include proteins having an amino acid sequence sharing at least about 70% amino acid sequence identity with the novel isoforms of the invention, preferably at least about 75%, more preferably at least about 80%, still more preferably at least about 85%, even more preferably at least about 90%, and most preferably at least about 95%. Amino acid substitutions are preferably substitutions of single amino-acid residues.

“Identity” or “homology” with respect to the variant polypeptides, detailed above, is defined herein as the percentage of amino acid residues in the candidate sequence (variant) that are identical with the residues in the reference protein, e.g., SEQ ID NO:19, after aligning the sequences and introducing gaps, if necessary, to achieve the maximum percent homology, and not considering any conservative substitutions as part of the sequence identity. No N- nor C-terminal extensions, deletions nor insertions shall be construed as reducing identity or homology.

Parameters for polypeptide sequence comparison include the following:

1) Algorithm: Needleman and Wunsch, J. Mol. Biol. 48: 443-453 (1970) Comparison matrix: BLOSSUM62 from Hentikoff and Hentikoff, Proc. Natl. Acad. Sci. USA. 89:10915-10919 (1992)

Gap Penalty: 12

Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Polypeptide embodiments further include an isolated polypeptide comprising a polypeptide having at least a 50, 60, 70, 80, 85, 90, or 95% identity to a reference polypeptide, wherein the variant polypeptide sequence may be identical to the reference sequence of SEQ ID NO:19 or may include up to a certain integer number of amino acid alterations as compared to the reference sequence, wherein the alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein the number of amino acid alterations is determined by multiplying the total number of amino acids in the reference sequence, e.g., SEQ ID NO:19 by the integer defining the percent identity divided by 100 and then subtracting that product from the total number of amino acids in SEQ ID NO:19, or:

Na=Xa−(Xa Y), wherein Na is the number of amino acid alterations, Xa is the total number of amino acids in SEQ ID NO:19, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xa and Y is rounded down to the nearest integer prior to subtracting it from Xa.

A variety of methodologies well-known to the skilled practitioner can be utilized to obtain isolated human T-type calcium channel α_{1I} subunit molecules detailed herein, e.g., α_{1I-1} subunit. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating human T-type calcium channel α_{1I-1} subunit polypeptides. These include, but are not limited to, immunochromatography, HPLC, size-exclusion chromatography, ion-exchange chromatography and immune-affinity chromatography.

A fragment or a biologically active or equivalent fragment of the novel isoform proteins of the invention are also embraced by the invention. Such a “fragment” is meant to refer to a protein which contains a portion of the complete amino acid sequence of the reference protein, e.g., SEQ ID NO:19. Such molecules are expected to have, inter alia, similar biological functions/properties equivalent to the polypeptide from which the fragment is derived.

Preferred fragments are those which retain a distinct functional capability of the corresponding reference isoforms of the invention from which the fragment is derived. Exemplary functions include voltage regulated calcium influx. Other functional capabilities which can be retained in a fragment of either of the novel isoforms of the invention include the ability to interact with antibodies as well as with other polypeptides (such as other subunits of the human T-type calcium channel) and binding partners. Those skilled in the art are well versed in methods for selecting fragments which retain a functional capability of the reference variant. Functional capability of the target fragment can be confirmed by synthesizing the fragment and testing of the capability according to standard methods. For example, when testing the fragment for its ability to voltage regulate calcium influx, a skilled artisan may transform and express the target fragment in a cell in which calcium influx can be measured. Such methods, are standard in the art.

The variant polypeptides of the invention polypeptide, functional equivalents thereof, and biologically active fragments, can also be produced by chemical synthesis. For example, synthetic polypeptides can be produced using Applied Biosystems, Inc. Model 430A or 431A automatic peptide synthesizer (Foster City, Calif.) employing the chemistry provided by the manufacturer.

The present invention also provides compositions containing an acceptable carrier and any of an isolated, purified novel isoforms of the invention, an active fragment thereof, or a purified, mature protein and active fragments thereof, alone or in combination with each other. These polypeptides or proteins can be recombinantly derived, chemically synthesized or purified from native sources.

Antibodies and Uses Therefor—The variant polypeptides of the invention or a immunologically active fragment/antigenic peptides thereof may be used as immunogens to generate an antibody or a functionally active antibody fragment specific for one of the variant polypeptides disclosed herein. (See, for example, Antibodies: A Laboratory Manual ed. by Harlow and Lane (Cold Spring Harbor Press: 1988)). The subject antibodies, in turn, can be used for producing hybridoma(s), and identifying pharmaceutical compositions, and for studying DNA/protein interaction.

The antigenic peptide fragment of the herein disclosed human T-type calcium channel α_{1I} isoforms, e.g., an α_{1I-1} subunit comprises at least 8 amino acid residues of the amino acid sequence shown in SEQ ID NO:19 and encompasses an epitope of the subject α_{1I-1} such that an antibody raised against the peptide forms a specific immune complex with the subject α_{1I-1} from which it was derived. Preferably, the antigenic peptide comprises at least 10 amino acid residues, more preferably at least 15 amino acid residues, even more preferably at least 20 amino acid residues, and most preferably at least 30 amino acid residues. Methods of determining the antigenic determinant of an antibody or peptide fragment thereof are known and may be employed to obtain synthetic antigenic peptides useful in accordance with the present invention. See, for example, U.S. Pat. No. 5,194,392 to Geyson et al., including the references cited therein.

Consequently, an embodiment of the invention contemplates an intact, fully human anti-human T-type calcium channel α_{1I-1} subunit monoclonal antibody in an isolated form or in a pharmaceutical preparation. Methods for developing a monoclonal antibody that interacts with and inhibits the activity of the subject human T-type calcium channel α_{1I} subunit are well known. A representative method of developing such an antibody is provided herein, it being understood that this method is provided for illustrative purposes only.

The term “antibody” is used in the broadest sense and specifically covers single monoclonal antibodies (including agonist and antagonist antibodies) and antibody compositions with polyepitopic specificity. In general, the term refers to immunoglobulin molecules and antigenically or immunologically active fragments of immunoglobulin molecules, i.e., molecules that contain an antigen binding site which specifically binds (immunoreacts with) an antigen, such as for example, the α_{1I-1} subunit polypeptide. It is understood that the term “antibody” includes not only intact immunoglobulin molecules but also the well-known active fragments F(ab′)2, and Fab. F(ab′)2, and Fab fragments which lack the Fc fragment of intact antibody, clear more rapidly from the circulation, and may have less non-specific tissue binding of an intact antibody (Wahl et al., J. Nucl. Med. 24:316-325 (1983)).

Methods of making antibodies are well known. For example, polyclonal anti-α_{1I-1} subunit antibodies can be prepared by immunizing a suitable subject with an α_{1I-1} subunit immunogen. Various hosts including goats, rabbits, rats, mice, humans, and others may be immunized by injection with α_{1I-1} subunit or an immunologically active fragment(s) thereof for producing the antibodies. Alternatively, short stretches of α_{1I-1} subunit amino acids may be fused with those of another protein to form chimeric entities, and antibodies to the chimeric entity may then produced. At an appropriate time after immunization, e.g., when the anti-α_{1I-1} subunit antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. A common technique entails fusing an immortal cell line (typically a myeloma) to lymphocytes (typically splenocytes) from a mammal immunized with an α_{1I-1} subunit immunogen as described above, followed by screening the culture supernatants of the resulting hybridoma cells to identify a hybridoma producing a monoclonal antibody that binds a α_{1I-1} subunit.

Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynabacterium parvum are especially preferable.

An appropriate immunogenic preparation can contain, for example, recombinantly expressed α_{1I-1} subunit protein or a chemically synthesized α_{1I-1} subunit protein. The preparation can further include an adjuvant, such as Freund's complete or incomplete adjuvant, or similar immunostimulatory agent. Immunization of a suitable subject with an immunogenic α_{1I-1} subunit preparation induces a polyclonal anti-α_{1I-1} subunit antibody response.

The prior art is replete with methods of preparing human monoclonal antibodies. Representative methods are disclosed in U.S. Pat. No. 5,567,610, issued to Borrebaeck et al., U.S. Pat. No. 5,565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, new York, 1987), and Boerner et al., J. Immunol., 147: 86-95 (1991). As well, such antibodies may also be prepared by immunizing transgenic animals that are capable of producing human antibodies (e.g., Jakobovits et al., Proc. Nat'l. Acad. Sci. USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

One well versed in the art of immunology is well aware that only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). For example, it is well accepted that the pFc′ and Fc regions of an antibody are not involved in antigen binding but instead play a significant role in the complement cascade. Significantly, an antibody from which the pfc′ region has been enzymatically cleaved, or which has been produced without the pFc′ region, designated an F(ab′)₂ fragment, retains both of the antigen binding sites of an intact antibody. Likewise, an antibody from which the Fc region has been enzymatically cleaved, or which has been produced without the Fc region, designated an Fab fragment, retains one of the antigen binding sites of an intact antibody molecule. Fab fragments are composed of a covalently bound antibody light chain and a portion of the antibody heavy chain denoted Fd, which is the major determinant of antibody specificity (a single Fd fragment may be associated with up to ten different light chains without altering antibody specificity) and Fd fragments retain epitope-binding ability in isolation.

As well, it is well accepted that within the antigen-binding portion of an antibody, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three (3) complementarity determining regions (CDR1 through CDR3). The CDRs, and in particular the CDR3 regions, and more particularly the heavy chain CDR3, are largely responsible for antibody specificity.

Conventional immunoassays may be employed to identify antibodies having the desired specificity. Such protocols include but are not limited to those described in U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, N.Y. (1988), each incorporated by reference herein. Likewise, the sequences of the antigen-binding sites of an anti-α_{1I-1} subunit monoclonal antibodies once identified by any one or more of the above referenced assays can be determined using amino acid sequencing techniques that are routine in the art. The same can be done with the relevant FR and CDR regions.

A skilled artisan well versed in immunology is also aware that one may increase the specificity/affinity/avidity of an antibody for its specific antigen by “humanizing” the subject antibody. Techniques to humanize antibodies are also well known to one skilled in the art of immunology. The technique is particularly useful when non-human animal (e.g., murine) antibodies which inhibit a target human T-type calcium channel subunit, e.g., the α_{1I-1} subunit of the invention are identified. It is also known that non-CDR regions of a mammalian antibody may be replaced with corresponding regions of non-specific or hetero-specific antibodies while retaining the epitope specificity of the original antibody is well known. This method proposes covalently joining non-human CDRs (e.g., murine) to human FR and/or Fc/pFc′ regions to produce a functional antibody. The resulting “humanized” antibodies can be used in the treatment of a human subject in the methods according to the invention. The proposed chimeric and humanized monoclonal antibodies can also be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060. An example of a method for humanizing a murine antibody is provided in U.S. Pat. Nos. 5,530,101 and 5,777,085 to Queen et al.—PDL. See also PCT International Publication No. WO 92/04381 which teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Reportedly, the above described humanized antibodies are capable of provoking an immune response against the non-human sequences (HAMA response). Consequently, such antibodies are not preferred for use in immuno-compromised humans and are particularly not preferred for extended use. However, the HAMA response may be lessened or abolished by techniques well known to one skilled in the art of immunology. Indeed, the prior art is replete with means of lessening or abolishing the HAMA response. Preferably, the chimeric antibodies of the invention are fully human monoclonal antibodies, where some or all of the FR regions of the antibody are replaced by other homologous human FR regions. A particular utility of the fully human chimeric antibodies is that they will not evoke an immune response against the antibody itself.

In accordance with the above, an embodiment of the invention provides F(ab′)₂, and Fab fragments of an anti-α_{1I-1} subunit monoclonal antibody; chimeric antibodies in which the Fc and/or FR and/or CDR1 and/or CDR2 and/or light chain CDR3 regions of the anti-α_{1I-1} subunit antibody have been replaced by homologous human or non-human sequences; chimeric F(ab′)₂ fragment antibodies in which the FR and/or CDR1 and/or CDR2 and/or light chain CDR3 regions of an anti-α_{1I-1} subunit antibody have been replaced by homologous human or non-human sequences; and chimeric Fab fragment antibodies in which the FR and/or CDR1 and/or CDR2 and/or light chain CDR3 regions have been replaced by homologous human or non-human sequences. It is understood that a skilled artisan may alter an anti-α_{1I-1} subunit antibody by CDR grafting or produce chimeric antibodies or antibody fragments containing, all or part thereof, of the disclosed heavy and light chain V-region CDR amino acid sequences (Jones et al., Nature 321:522, 1986; Verhoeyen et al., Science 39:1534, 1988 and Tempest et al., Bio/Technology 9:266, 1991), without destroying the specificity of the antibodies for either one of the human T-type calcium channel isoforms of the invention. Such CDR grafted or chimeric antibodies or antibody fragments can be very effective in inhibiting human T-type calcium channel α_{1I-1} subunit activity in animals (e.g. primates) and humans.

Fab fragments, including chimeric Fab fragments, are preferable in those methods where the antibodies of the invention are administered directly to a local tissue environment. For example, the Fab fragments are preferred when the antibody of the invention is administered directly to the brain. Fabs offer several advantages over F(ab′)₂ and whole immunoglobulin molecules for this therapeutic modality. That Fab molecules of the invention are preferable for the above purposes becomes evident when one considers that since Fabs have only one binding site for their cognate antigen, the formation of immune complexes is precluded in contrast to the generation of such complexes when using bivalent F(ab′)₂ s and whole immunoglobulin molecules upon their encounter with the target antigen. Of equal import is the observation that immune complex deposition in tissues can produce adverse inflammatory reactions. As well, the absence of a Fc region in Fabs prevents an adverse inflammatory reactions that generally attends antibodies with an Fc, whose activation, in turn, may activate the complement cascade. Also, it is believed that tissue penetration and bioavailability of the small Fab molecule is likely to be much better than that of the larger whole antibody. More, it is cheaper and easier to produce Fabs, e.g., in bacteria, such as E. coli, whereas whole immunoglobulin antibody molecules require mammalian cells for their production in useful amounts. Indeed, production of Fabs in E. coli makes it possible to produce these antibody fragments in large fermenters which are less expensive than cell culture-derived products. For inoculation or prophylactic use, the antibodies of the present invention should preferably be intact antibody molecules including the Fc region. Such intact antibodies generally exhibit longer half-lives than smaller fragment antibodies (e.g. Fab) and are more suitable for intravenous, intraperitoneal, intramuscular, intracavity, subcutaneous, or transdermal administration.

In addition to the above, smaller antibody fragments and small binding peptides having binding specificity for a human T-type calcium channel α_{1I} isoform of the invention will find use as inhibitors of human T-type calcium channel α_{1I} activity and are thus also embraced by the invention. For example, single-chain antibodies can be constructed in accordance with the methods described in U.S. Pat. No. 4,946,778 to Ladner et al. Such single-chain antibodies include the variable regions of the light and heavy chains joined by a flexible linker moiety. Methods for obtaining a single domain antibody (“Fd”) which comprises an isolated VH single domain are detailed in Ward et al., Nature 341:644-646 (1989)).

Methods for assessing the affinity of antibodies for an α_{1I} subunit protein isoform, e.g., α_{1I-1} subunit are also well known. For example, Scatchard analysis in conjunction with radioimmunoassay techniques may be used to assess the affinity of antibodies of the invention. Affinity is expressed as an association constant, Kₐ, which is defined as the molar concentration of α_{1I-1} subunit-antibody complex divided by the molar concentrations of free antigen and free antibody under equilibrium conditions. The Kₐ determined for a preparation of polyclonal antibodies, which are heterogeneous in their affinities for multiple α_{1I-1} subunit epitopes, represents the average affinity, or avidity, of the antibodies for α_{1I-1} subunit. The Kₐ determined for a preparation of monoclonal antibodies, which are monospecific for a particular α_{1I-1} subunit epitope, represents a true measure of affinity. High-affinity antibody preparations with Kₐ ranging from about 10⁹ to 10¹² l/mole are preferred for use in immunoassays in which the α_{1I-1} subunit-antibody complex must withstand rigorous manipulations. Low-affinity antibody preparations with Kₐ ranging from about 10⁶ to 10⁷ l/mole are preferred for use in immunopurification and similar procedures which ultimately require dissociation of α_{1I-1} subunit, preferably in active form, from the antibody (Catty, D. (1988) Antibodies, Volume I: A Practical Approach, IRL Press, Washington D.C.; Liddell, J. E. and Cryer, A. (1991) A Practical Guide to Monoclonal Antibodies, Johne Wiley & Sons, New York N.Y.).

Immunological procedures useful for in vitro detection of an α_{1I} isoform, e.g., α_{1I-1} (SEQ ID NO:19) in a sample are well known. Indeed, numerous protocols for competitive binding or immunoradiometric assays using either polyclonal or monoclonal antibodies with established specificities are known to one skilled in the art. Such immunoassays include, for example, ELISA, Pandex microfluorimetric assay, agglutination assays, flow cytometry, serum diagnostic assays and immunohistochemical staining procedures, which are well known in the art. Typically, the immunoassay involves the measurement of complex formation between the target α_{1I} isoform subunit and its specific antibody. A two-site, monoclonal-based immunoassay utilizing monoclonal antibodies reactive to two non-interfering α_{1I-1} subunit epitopes is preferred, but a competitive binding assay may also be employed. The antibodies may be made detectable by various means well known in the art. For example, a detectable marker can be directly or indirectly attached to the antibody. Useful markers include, for example, radionuclides, enzymes, fluorogens, chromogens and chemiluminescent labels.

III. Proposed uses for the human T-type calcium channel α_{1I-1} subunit isoform polypeptide isoforms and polynucleotide encoding same.

The invention as described herein is useful for a variety of therapeutic and non-therapeutic purposes. For example, the invention permits isolation of an isoform of a human T-type calcium channel α_{1I} subunit polypeptide molecule, e.g., α_{1I-1} subunit polypeptide containing the amino acid sequence of SEQ ID NO:19, by for example expression of a recombinant nucleic acid to produce large quantities of the desired polypeptide which may be isolated using standard protocols. Likewise, polynucleotides comprising a nucleotide sequence depicted in SEQ ID NO:18 or a sequence substantially similar to SEQ ID NO:18 may also be isolated using the polynucleotides disclosed herein.

A wide variety of labels and conjugation techniques are known by those skilled in the art and may be used in various nucleic acid and amino acid assays. Means for producing labeled hybridization or PCR probes for detecting sequences related to polynucleotides encoding, for example, the α_{1I-1} subunit polypeptide (SEQ ID NO:19) include oligolabeling, nick translation, end-labeling, or PCR amplification using a labeled nucleotide. Alternatively, the sequences encoding the α_{1I-1} subunit polypeptide (SEQ ID NO:19), or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

As such, portions or fragments of the cDNA sequences identified herein (and the corresponding complete gene sequences) can be used in numerous ways as polynucleotide reagents. For example, these sequences can be used to: (i) detect α_{1I-1} subunit encoding nucleotide sequences in a sample; and (ii) map their respective genes on a chromosome, and, thus, locate gene regions associated with genetic disease. Gene products can likewise be used as herein described.

The binding or interaction of a candidate compound with a receptor or fragments thereof e.g., α_{1I-1} can also be measured directly by using radioactively labeled compound of interest or by the second messenger effect resulting from the interaction or binding of the candidate compound. (See, e.g., Lazereno and Birdsall (1993) Br. J. Pharmacol. 109:1120-1127.) Modulation in receptor signaling can be measured using a detectable assay, e.g., the FLIPR assay. (See, e.g., Coward, P. (1999) Anal. Biochem. 270:242-248; Sittampalam, supra; and Gonzalez and Negulescu, supra.) Activation of certain receptors, in particular, GPCRs, can be measured by an ³⁵S-GTPγS binding assay. (See, e.g., Lazareno (1999) Methods Mol. Biol. 106:231-245.) Alternatively, the candidate compounds can be subjected to competition screening assays, in which a known ligand, preferably labeled with an analytically detectable reagent, most preferably radioactivity, is introduced with the drug to be tested and the capacity of the compound to inhibit or enhance the binding of the labeled ligand is measured. Compounds are screened for their increased affinity and selectivity for the specific receptor or fragments thereof. Transcription based assays that identify signals that modulate the activity of cell surface proteins, e.g., receptors, ion channels, etc., may be used to screen candidate compounds for their ability to stimulate reporter gene product expression and their potential to stimulate the expression of a human T-type calcium channel α_{1I} isoform, e.g., α_{1I-1}. Transcription-based assays are well known in the art. (See, e.g., Zlokarnik, et al. (1998) Science 279:84-88; Siverman, supra; and Gonzalez and Negulescu, (1998) Curr. Opin. Biotechnol. 9:624-631.) These transcription based assays assess the intracellular transduction of an extracellular signal using recombinant cells that are modified by introduction of a reporter gene under the control of a regulatable promoter.

Determining the ability of an α_{1I} isomer of the invention,—the α_{1I-1} subunit polypeptide (SEQ ID NO:19) to bind to or interact with an α_{1I-1} target molecule (binding partner/binding peptide) can be accomplished, for example, by coupling the α_{1I-1} subunit with a radioisotope or enzymatic label such that binding of the α_{1I-1} subunit to an α_{1I-1} target molecule can be determined by detecting the labeled α_{1I-1} subunit in a complex. See, e.g., Wainscott et al. (1993) Mol. Pharmacol. 43:419-426; and Loric, et al. (1992) FEBS Lett. 312:203-207.) For example, the α_{1I-1} subunit, can be labeled with 125I, 35S, 14C, or 3H, either directly or indirectly, and the radioisotope detected by direct counting of radioemmission or by scintillation counting. Alternatively, α_{1I-1} subunit molecules can be enzymatically labeled with, for example, horseradish peroxidase, alkaline phosphatase, or luciferase, and the enzymatic label detected by determination of conversion of an appropriate substrate to product.

A two-hybrid system-based approach can also be employed for compound screening, small molecule identification, and drug discovery. The underlying premise of the two-hybrid system, originally described in yeast by Fields and Song (1989) Nature 340:245-246, provides a connection between a productive protein-protein or protein-compound interaction pair of interest and a measurable phenotypic change in yeast. A reporter cassette containing an up-stream activation sequence which is recognized by a DNA binding domain, is operationally linked to a reporter gene, which when expressed under the correct conditions will generate a phenotypic change. The original two-hybrid system has recently been modified for applicability in high-throughput compound screening. (See, e.g., Ho et al. (1996) Nature 382:822-826; Licitra and Liu (1996) Proc. Natl. Acad. Sci. USA 93:12817-12821; and Young et al. (1998) Nature Biotech. 16:946-950.)

The invention also features binding peptides (agents) that bind to the α_{1I} isoforms disclosed herein. These peptides may be derived from a variety of sources. For example, binding peptides may easily be synthesized or produced by recombinant means by those of skill in the art. Using routine procedures known to those of ordinary skill in the art, one can determine whether a peptide which binds to a human T-type calcium channel α_{1I-1} subunit isoform is useful according to the invention by determining whether the peptide inhibits the activity of human T-type calcium channel α_{1I-1} subunit in a voltage gated calcium influx assay, as discussed above.

The binding peptides according to the invention may also be an antibody or a functionally active antibody fragment. Antibodies are well known to those of ordinary skill in the science of immunology. Such fragments are also well known in the art and are regularly employed both in vitro and in vivo. In particular, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983)).

The invention also embraces agents which bind selectively to the one of the herein disclosed human T-type calcium channel α_{1I} subunit polypeptide isoform, e.g., SEQ ID NO:19 or SEQ ID NO:21 and agents which bind preferentially to either one or both of the isoforms disclosed herein. Agents which bind to the isoforms of SEQ ID NOS:19 or 21 and fragments of the aforementioned polypeptides and nucleic acid molecules are also included within the scope of the invention.

Selective binding means that the agent binds to, for example, a the human T-type calcium channel α_{1I-1} subunit but not to non-α_{1I-1} or α_{1I-2} (i.e., those subunits which do not have or encode SEQ ID NOS:19 or 21). Preferential binding means that the agent binds more to the human T-type calcium channel α_{1I-1} (SEQ ID NO:19) or α_{1I-2} subunit (SEQ ID NO:21) than to human T-type calcium channel non-α_{1I} subunit, e.g., the agent binds with greater affinity or avidity to the human T-type calcium channel α_{1I-1} subunit isoforms disclosed herein. The agents can inhibit or increase activity of the respective isoform polypeptide—(antagonists and agonists, respectively).

In order to determine whether an α_{1I} isoform specific binding peptide binds to, e.g., the α_{1I-1} subunit any known binding assay may be employed. For example, the peptide may be immobilized on a surface and then contacted with a labeled α_{1I-1} subunit. The amount of α_{1I-1} subunit which interacts with the binding peptide or the amount which does not bind to the α_{1I-1} subunit binding peptide may then be quantitated to determine whether the binding peptide binds to the subject α_{1I-1} subunit. Further, the binding of the α_{1I-1} subunit and a non-α_{1I-1} subunit can also be compared to determine if the binding peptide binds selectively or preferentially.

Consequently, in screening for modulators of the α_{1I-1} subunit (SEQ ID NO:19) it is preferred that compounds (e.g. synthetic combinatorial libraries, natural products, peptide libraries, etc.) are tested for modulation of α_{1I-1} subunit activity at a variety of voltages which cause partial or complete membrane depolarization, or hyperpolarization. These assays are conducted according to standard procedures of testing calcium channel function (e.g. patch clamping, fluorescent Ca²⁺ influx assays) which require no more than routine experimentation. Using such methods, modulators of all subunit activity which are active at particular voltages (e.g. complete membrane depolarization) can be identified. Such compounds are useful for selectively modulating calcium channel activity in conditions which may display voltage dependence.

Other assays for identifying compounds that modulate ion channel activity are practiced by measuring the ion channel activity when a cell expressing the ion channel of interest, or fragments thereof, is exposed to a solution containing the test compound and a ion channel selective ion and comparing the measured ion channel activity to the native ion channel activity of the same cell or a substantially identical control cell in a solution not containing the test compound. Methods for practicing such assays are known to those of skill in the art. (See, e.g., Mishina et al. (1985) Nature 313:364-369; and Noda, et al. Nature 322:836-828.)

In addition, an aspect of the invention is drawn to the use of the novel nucleic acid molecules to map the location of the gene on a chromosome. This process is called chromosome mapping. Probes derived from the novel polynucleotides sequences disclosed herein can be used herein to identify genomic or cDNA library clones possessing an identical or substantially similar nucleic acid sequence. A suitable oligonucleotide or set of oligonucleotides, which is capable of hybridizing under stringent hybridization conditions to the desired sequence, a variant or fragment thereof, or an anti-sense complement of such an oligonucleotide or set of oligonucleotides, can be synthesized by means well known in the art (see, for example, Synthesis and Application of DNA and RNA, S. A. Narang, ed., 1987, Academic Press, San Diego, Calif.) and employed as a probe to identify and isolate the desired sequence, variant or fragment thereof by techniques known in the art. Techniques of nucleic acid hybridization and clone identification are disclosed by Sambrook, et al., Molecular Cloning, A Laboratory Manual, 2d ed., Cold Spring Harbor Laboratory Press, Plainview, N.Y. (1989), and by Hames, B. D., et al., in Nucleic Acid Hybridization, A Practical Approach, IRL Press, Washington, D.C. (1985). Means for producing specific hybridization probes for detecting identical or related sequences are well known. mRNA probes may also be used, whose method of preparation is well known to a person well versed in the art of cell biology. Vectors for the production of mRNA probes are commercially available, and may be used to synthesize RNA probes in vitro by means of the addition of the appropriate RNA polymerases and the appropriate labeled nucleotides.

To facilitate the detection of a desired nucleic acid sequence, or variant or fragment thereof, whether for cloning purposes or for the mere detection of the presence of the sequence, the above-described probes may be labeled with a detectable group. Such a detectable group may be any material having a detectable physical or chemical property. Such materials have been well-developed in the field of nucleic acid hybridization and, in general, most any label useful in such methods can be applied to the present invention.

In one aspect, the invention proposes the use of the hybridization probes for mapping the naturally occurring genomic sequence. Fragments of the α_{1I} isoform encoding sequences (SEQ ID NOS:18 or 20) may be used to map these genes to the appropriate mouse and human chromosomes. The sequences may be mapped to a particular chromosome, to a specific region of a chromosome or to artificial chromosome constructions, such as human artificial chromosomes (HACs), yeast artificial chromosomes (YACs), bacterial artificial chromosomes (BACs), bacterial P1 constructions, or single chromosome cDNA libraries. (See, e.g., Price, C. M. (1993) Blood Rev. 7:127-134, and Trask, B. J. (1991) Trends Genet. 7:149-154.)

Briefly, the sequence(s) is specifically targeted to and can hybridize with a particular location on an individual human chromosome. The mapping of relevant sequences to chromosomes according to the present invention is an important first step in correlating those sequences with gene associated disease. Upon mapping a sequence to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. Nature, 325:783-787 (1987). The differences in the cDNA or genomic sequence between affected and unaffected individuals can also be determined. If a mutation is observed in some or all of the affected individuals but not in any normal individuals, then the mutation is likely to be the causative agent of the disease.

For example, α_{1I-1} subunit encoding genes can be mapped to chromosomes by preparing PCR primers (preferably 15-25 bp in length) from the herein disclosed α_{1I-1} nucleotide sequence. Computer analysis of the α_{1I-1} subunit sequences can be used to predict primers that do not span more than one exon in the genomic DNA, thus complicating the amplification process. Thereafter, these primers can be used for PCR screening of somatic cell hybrids containing individual human chromosomes. Only those hybrids containing the human gene corresponding to the α_{1I-1} subunit sequences will yield an amplified fragment.

Somatic cell hybrids containing only fragments of human chromosomes can be produced by using human chromosomes with translocations and deletions. PCR mapping of somatic cell hybrids is a rapid procedure for assigning a particular sequence to a particular chromosome. Three or more sequences can be assigned per day using a single thermal cycler. Using the α_{1I-1} subunit nucleotide sequences to design oligonucleotide primers, sublocalization can be achieved with panels of fragments from specific chromosomes.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel will contain either a single human chromosome or a small number of human chromosomes, and a full set of mouse chromosomes, allowing easy mapping of individual genes to specific human chromosomes. (D'Eustachio P. et al. (1983) Science 220:919-924).

Fluorescent in situ hybridization (FISH) may be correlated with other physical chromosome mapping techniques and genetic map data. (See, e.g., Verma et al. (1988) Human Chromosomes: A Manual of Basic Techniques, Pergamon Press, New York, N.Y.) Examples of genetic map data can be found in various scientific journals or at Online Mendelian Inheritance in Man (OMIM). Correlation between the location of the gene encoding α_{1I-1} on a physical chromosomal map and a specific disease, or predisposition to a specific disease, may help delimit the region of DNA associated with that genetic disease. The nucleotide sequences of the subject invention may be used to detect differences in gene sequences between normal, carrier, or affected individuals. Chromosome spreads can be made using cells whose division has been blocked in metaphase by a chemical such as colcemid that disrupts the mitotic spindle. Consequently, fluorescence in situ hybridization (FISH) of a DNA sequence to a metaphase chromosomal spread can further be used to provide a precise chromosomal location in one step.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

More, differences in the DNA sequences between individuals affected and unaffected with a disease associated with a human T-type calcium channel α_{1I} subunit or an isoform thereof such as the ones disclosed herein can also be determined. If a mutation is observed in some or all of the affected individuals but not in any unaffected individuals, then it is save to assume that the mutation is likely the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Other mapping strategies include in situ hybridization (described in Fan, Y. et al. Proc. Natl. Acad. Sci. USA, 87:6223-27 (1990)), pre-screening with labeled flow-sorted chromosomes, and pre-selection by hybridization to chromosome specific cDNA libraries. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region (see, e.g., Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

To detect nucleic acid encoding a polypeptide of the invention or one having the sequence substantially as set forth in SEQ ID NO:18, standard hybridization and/or PCR techniques may be employed using a nucleic acid probe or a PCR primer. Suitable probes and primers may be designed by those of ordinary skill in the art based on the α_{1I} isoform subunit cDNA sequences provided herein.

Consequently, determining the level of, e.g., α_{1I-1} subunit (SEQ ID NO:18) expression may be accomplished by Northern blot analysis. Polyadenylated [poly(A)+] mRNA is isolated from a test sample. The mRNA is fractionated by electrophoresis and transferred to a membrane. The membrane is probed with labeled α_{1I-1} subunit cDNA. In another embodiment, α_{1I-1} subunit expression is measured by quantitative PCR applied to expressed mRNA. In yet another embodiment, the test reagent is incubated with a cell transfected with an α_{1I-1} subunit polynucleotide expression vector, and the effect of the test reagent on α_{1I-1} subunit transcription is measured by Northern blot analysis, as described above.

In another embodiment of the invention, the polynucleotides encoding a novel isoform of the invention, or any fragment or complement thereof, may be used for therapeutic purposes. As well, complements of the human T-type calcium channel α_{1I} subunit isoform encoding nucleic acid molecules can be useful as antisense oligonucleotides, e.g., by delivering the antisense oligonucleotide to an animal to induce a human T-type calcium channel α_{1I-1} subunit “knockout” phenotype. The administration of antisense RNA probes to block gene expression is discussed in Lichtenstein, C., Nature 333:801-802 (1988).

Thus, an aspect of the invention features using the complement of the polynucleotide encoding a novel isoform of the invention in situations in which it would be desirable to block the transcription of the mRNA. In particular, cells may be transformed with sequences complementary to polynucleotides encoding a novel isoform of the invention. Thus, complementary molecules or fragments may be used to modulate α_{1I} subunit protein isoforms, e.g., α_{1I-1} of SEQ ID NO:18 activity, or to achieve regulation of gene function. Such technology is now well known in the art, and sense or antisense oligonucleotides or larger fragments can be designed from various locations along the coding or control regions of sequences encoding α_{1I} subunit isoform of the invention, e.g., α_{1I-1}.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors which will express nucleic acid sequences complementary to the polynucleotides of the gene encoding any one or both of the α_{1I} subunit isoform detailed herein. See, e.g., Sambrook, supra: and Ausubel, supra.

For example, genes encoding an α_{1I-1} subunit detailed herein can be turned off by transforming a cell or tissue with expression vectors which express high levels of a polynucleotide, or fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding α_{1I}.subunit isoform detailed herein, e.g., SEQ ID NO:18. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing Co., Mt. Kisco, N.Y., pp. 163-177.) A complementary sequence or antisense molecule may also be designed to block translation of mRNA by preventing the transcript from binding to ribosomes.

Any of the therapeutic methods described herein may be applied to any subject in need of such therapy, including, for example, mammals such as dogs, cats, cows, horses, rabbits, monkeys, and most preferably, humans.

Another aspect of the invention is directed to the use of synthetic oligonucleotides, or other antisense chemical structures designed to bind to mRNA encoding either one of the herein disclosed α_{1I} isoforms or a equivalent nucleic acid molecule having a sequence of nucleotides substantially the same as detailed herein (SEQ ID NOS:18 or 20) and inhibit translation of mRNA thereby inhibiting expression of encoded gene product. Significantly, such antisense molecules will allow the skilled artisan the means to alter levels of expression of either on of the novel isoform polypeptides or a structurally related polypeptide in a suitable host cell.

To be effective, the SAO may be designed so as to be able to pass through cell membranes in order to enter the cytoplasm of the cell. This is made possible by virtue of physical and chemical properties of the SAO which render it capable of passing through cell membranes (e.g. by designing small, hydrophobic SAO chemical structures) or by virtue of specific transport systems in the cell which recognize and transport the SAO into the cell. Likewise, the SAO can be designed so that upon administration it is directed to selected cell populations. For example, the SAO may be designed to bind to the α_{1I-1} subunit sequence which are found only in certain cell types. Alternatively, the SAO may be designed so that it only recognizes and thus selectively binds to the target mRNA sequence, comprising a nucleotide sequence corresponding to a nucleotide sequence contained, for example, in the sequence set forth in SEQ ID NO:18. This object is achieved by virtue of complementary base pairing to the mRNA. The SAO may also be designed to inactivate the target mRNA sequence by any one or more of three mechanisms—(a) binding to the target mRNA and thus inducing degradation of the mRNA by intrinsic cellular mechanisms such as RNAse H digestion; (b) inhibiting translation of the target mRNA by interfering with the binding of translation-regulating factors or of ribosomes; or (c) inclusion of other chemical structures, such as ribozyme sequences or reactive chemical groups sufficient to either degrade or chemically modify the target mRNA. Significantly, synthetic antisense oligonucleotide drugs capable of successfully achieving the above referenced objects via modulating mRNA targets are detailed in the prior art. See, e.g., Cohen (1989) Trends in Pharm. Sci. 10:435; and Weintraub (1990) Sci. Am. 262:40-46. Also see, Sarver et al. (1990) Science 247:1222, have reported the successful coupling of ribozymes to antisense oligonucleotides as a promising strategy for inactivating target mRNA.

A vector expressing the complement of the polynucleotide encoding the α_{1I-1} subunit polypeptide isoform may be administered to a subject to treat or prevent any one of the disorders described elsewhere in this application.

In accordance with the above, an aspect of the invention is drawn to a human T-type calcium channel α_{1I} isoform inhibitor, e.g., α_{1I-1} wherein the inhibitor is an antisense oligonucleotide that selectively binds to the α_{1I} isoform encoding nucleic acid molecule, e.g., the nucleic acid molecule of the invention or one having a nucleotide sequence that is substantially the same as those detailed herein, in an amount effective to reduce the expression of the encoded gene product. This is desirable in virtually any medical condition wherein a reduction of a human T-type calcium channel α_{1I} subunit activity is desirable, e.g., voltage gated calcium influx.

Alternatively, the isolation of the novel genes encoding the variant proteins of the invention may be used to detect and quantitate gene expression in biological samples e.g., biopsied tissues in which expression of an α_{1I} isoform, e.g., α_{1I-1} subunit encoding polynucleotides may be correlated with disease. Specifically, the isolation of the α_{1I} isoform(s) disclosed herein, makes it possible for the artisan to diagnose a disorder characterized by loss of expression or excessive expression of the either one of the α_{1I} isoforms disclosed herein. The polynucleotides for use in the diagnostic assays are preferably oligonucleotide sequences, complementary RNA and DNA molecules, and PNAs. The diagnostic assay may be used to distinguishing between absence, presence, and excess expression of to determine absence, presence, and excess expression of an α_{1I} isoform of the invention and to monitor regulation of the subject α_{1I} isoform levels during therapeutic intervention. In addition, the polynucleotides of the invention may also be used to determine the efficacy of a treatment protocol in a patient undergoing therapy for a one of the many human T-type calcium channel mediated pathologies that are responsive to treatment with the isoform encoding polynucleotides of the invention.

These methods involve determining expression of the nucleic acid molecule encoding said subunit polypeptide, e.g., SEQ ID NO:18, and/or the gene product thereof (SEQ ID NO:19). In the former situation, such determinations can be carried out via any standard nucleic acid determination assay, including the polymerase chain reaction, or assaying with labeled hybridization probes.

A representative diagnostic method detailing the use of the polynucleotide(s) for diagnostic purposes proposes utilizing PCR probes derived from the sequences detailed herein, for hybridization complexes as a means to identify nucleic acid sequences which encode a protein of SEQ ID NO:19 or a biologically equivalent protein. The probes are capable of detecting polynucleotide sequences having a sequence that is substantially the same as those detailed herein, including genomic sequences. Further, the probes of the invention may also be used for detecting related sequences, and should preferably contain at least 50% of the nucleotides from any of the α_{1I-1} encoding sequences. In the method, the specificity of the probe, and/or the stringency of the hybridization or amplification (maximal, high, intermediate, or low) will be determinative of whether the probe identifies only naturally occurring sequences encoding α_{1I-1} subunit polypeptide, alleles, or related sequences. The hybridization probes of the subject invention may be DNA or RNA and derived from the nucleotide sequence of SEQ ID NO:18 or from genomic sequence including promoter, enhancer elements, and introns of the naturally occurring α_{1I-1} subunit.

In order to provide a basis for the diagnosis of disease associated with aberrant expression of a human α_{1I} isoform of the invention, a “normal” or standard profile for expression is established. Normal levels may be obtained by combining body fluids or cell extracts taken from normal subjects, either animal or human, with a polynucleotide sequence of the invention or a fragment thereof, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from subjects who are symptomatic for disease. Deviation between standard and subject values is used to establish the presence of disease.

Alternatively, standard values for an α_{1I} subunit polypeptide expression, e.g., SEQ ID NO:19, can be established by, combining body fluids or cell extracts taken from normal mammalian subjects, preferably human, with an antibody to, for example, α_{1I-1} subunit of the invention, under conditions favoring the formation of complex therebetween. The amount of standard complex formation may be quantified by various methods, but preferably by photometric, means. Quantities of the α_{1I-1} subunit polypeptide expressed in control and disease samples from biopsied tissues or biological samples are compared with the standard values. Deviation between standard and subject values, in turn, will establish the parameters for diagnosing the disease.

A variety of protocols for detecting and measuring the expression of α_{1I-1} subunit polypeptide, using either polyclonal or monoclonal antibodies specific for the protein, are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on α_{1I-1} subunit polypeptide is preferred, but a competitive binding assay may be employed. These and other assays are well described in the art. (See, e.g., Hampton, R. et al. (1990) Serological Methods, a Laboratory Manual, APS Press, St Paul, Minn., Section IV; and Maddox, D. E. et al. (1983) J. Exp. Med. 158:1211-1216).

Methods for quantifying the expression of the target, e.g., polynucleotides or protein include radiolabeling or biotinylating nucleotides, co-amplification of a control nucleic acid, and standard curves onto which the experimental results are interpolated. (See, e.g., Melby, P. C. et al. (1993) J. Immunol. Methods, 159:235-244; and Duplaa, C. et al. (1993) Anal. Biochem. 212:229-236.) Formats are available that speed up the quantitation of multiple samples, e.g., running the assay in an ELISA format where the oligomer of interest is presented in various dilutions and a spectrophotometric or calorimetric response provides rapid quantitation.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

The present invention also provides various methods for determining whether a compound/agent can modulate the activity of at least one of the herein disclosed α_{1I} subunit polypeptide isoforms, e.g., α_{1I-1}. The compound can be a substantially pure compound of synthetic origin combined in an aqueous medium, or the compound can be a naturally occurring material such that the assay medium is an extract of biological origin, such as, for example, a plant, animal, or microbial cell extract. A general method proposes contacting α_{1I-1} subunit or fragments thereof, with the compound under suitable conditions and subsequently determining if the compound modulates the activity of the α_{1I-1} subunit. The compounds of interest can function as agonists or antagonists of α_{1I-1} subunit activity. α_{1I-1} subunit or fragments thereof, can be expressed on a cell or tissue, naturally or recombinantly, or immobilized by attachment to a solid substrate, e.g., nitrocellulose or nylon membrane, glass, beads, etc. Modulators of the α_{1I-1} subunit isoform of the invention may selectively inhibit or increase α_{1I-1} subunit polypeptide function based on the state of depolarization of the membrane with which the α_{1I-1} subunit is associated.

Also provided are efficient methods for identifying pharmacological agents or lead compounds for agents useful in the treatment of conditions associated with aberrant voltage gated calcium influx mediated by a human T-type calcium channel including the novel isoforms of the invention. Compounds and agents so identified are also within the scope of the invention.

A variety of assays for pharmacological agents are known, e.g., labeled in vitro protein binding assays, Ca²⁺ influx assays, etc. For example, calcium influx assays can be performed to screen and/or determine whether the α_{1I-1} subunit inhibitor has the ability to inhibit α_{1I-1} subunit activity, and whether the inhibition is selective. As an example, protein binding screens are routinely used to rapidly examine the binding of candidate pharmacological agents to a target protein, e.g., the α_{1I-1} subunit of the invention. The candidate pharmacological agents can be derived from, for example, combinatorial peptide libraries. Convenient reagents for such assays are known in the art.

The α_{1I} isoform polypeptides, for use in the methods of the invention are added to an assay mixture as an isolated polypeptide (where binding of a candidate pharmaceutical agent is to be measured) or as a cell or other membrane preparation containing a functional human T-type calcium channel subunit polypeptide. In the latter assay configuration, the cells contain the α_{1I} isoform, e.g., α_{1I-1} subunit as a preloaded polypeptide or as a nucleic acid (e.g. a cell transfected with an expression vector comprising the nucleotide sequence as shown in SEQ ID NO:18). The polypeptide can be produced recombinantly, or isolated from biological extracts, but preferably is synthesized in vitro. Polypeptides for use include the novel α_{1I} isoforms of the invention as well as chimeric proteins comprising a fusion of an α_{1I} isoform of the invention e.g., the α_{1I-1} subunit polypeptide with another polypeptide, e.g., a polypeptide capable of providing or enhancing protein-protein binding, or enhancing stability of the α_{1I-1} subunit polypeptide under assay conditions. A polypeptide fused to the α_{1I-1} subunit polypeptide or fragment thereof may also provide means of readily detecting the fusion protein, e.g., by immunological recognition or by fluorescent labeling.

The assay mixture further comprises a candidate pharmacological agent. Typically, a plurality of assay mixtures are run in parallel with different agent concentrations to obtain a different response to the various concentrations. Generally, one of these concentrations serves as a negative control, i.e., at zero concentration of agent or at a concentration of agent below the limits of assay detection. Candidate agents include numerous chemical classes, although typically they are organic compounds. Preferably, the candidate pharmacological agents are small organic compounds, i.e., those having a molecular weight of more than 50 yet less than about 2500. In instances, where the agent is a nucleic acid molecule, the agent typically is a DNA or RNA molecule, although modified nucleic acid molecules having non-natural bonds or subunits are also contemplated. Likewise, the agent may be an antibody composition.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The mixture composed of the foregoing assay materials is incubated under conditions whereby, but for the presence of the candidate pharmacological agent, the representative human T-type calcium channel α_{1I} subunit isoform of the invention transduces a control amount of voltage gated calcium influx. For determining the binding of a candidate pharmaceutical agent to the α_{1I-1} subunit, the mixture is incubated under conditions which permit binding. The order of addition of components, incubation temperature, time of incubation, and other parameters of the assay may be readily determined. Of significant import is the observation that such experimentation merely involves optimization of the assay parameters, not the fundamental composition of the assay. Following incubation, the level of voltage gated calcium influx or the level of specific binding between the α_{1I-1} subunit polypeptide and the candidate pharmaceutical agent is detected by any convenient method available to the user.

For cell free binding type assays, a separation step is often used to separate bound from unbound components. The separation step may be accomplished in a variety of ways. Although it is preferable that at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximize signal to noise ratios, primarily to minimize background binding, as well as for ease of separation and cost. Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet. See U.S. Patent Publication No. 2002/0147309 for details regarding factors that may effect separation.

Detection may be realized in any convenient way for cell-based assays such as a calcium influx assay, supra. The calcium influx resulting from voltage stimulus of the α_{1I-1} subunit polypeptide typically alters a directly or indirectly detectable product, e.g., a calcium sensitive molecule such as fura-2-AM.

For cell free binding assays, one of the components usually comprises, or is coupled to, a detectable label. A wide variety of labels can be used, such as those that provide direct detection (e.g., radioactivity, luminescence, optical or electron density, etc.) or indirect detection (e.g., epitope tag such as the FLAG epitope, enzyme tag such as horseradish peroxidase, etc.). The label may be bound to a human T-type calcium channel α_{1I-1} subunit polypeptide or the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

An exemplary cell-based assay of calcium influx involves contacting a neuronal cell expressing an α_{1I} isoform having a sequence of amino acids substantially as depicted in SEQ ID NO:19 or a biologically active fragment thereof or a structurally related protein, as determined by percent sequence identity of the primary sequence, e.g., 85% sequence identity and exhibiting a common calcium channel activity with a candidate pharmacological agent under conditions whereby the influx of calcium can be stimulated by application of a voltage to the test system, i.e., by membrane depolarization. Specific conditions are well known in the art and are described in Lin et al., Neuron 18:153-166, 1997. Examples of such methods are also described in U.S. Pat. No. 5,429,921. A reduction in the voltage gated calcium influx in the presence of the candidate pharmacological agent indicates that the candidate pharmacological agent reduces the induction of calcium influx of the all subunit in response to the voltage stimulus. An increase in the voltage gated calcium influx in the presence of the candidate pharmacological agent indicates that the candidate pharmacological agent increases the induction of calcium influx of the α_{1I} subunit in response to the voltage stimulus. Methods for determining changes in the intracellular calcium concentration are known in the art.

An amount of the α_{1I-1} subunit inhibitor which is effective to inhibit voltage gated calcium influx in the mammalian cell is an amount which is sufficient to reduce voltage gated calcium influx by at least 10%, preferably at least 20%, more preferably 30% and still more preferably 40%. An amount of a human T-type calcium channel α_{1I-1} subunit which is effective to increase voltage gated calcium influx in the mammalian cell is an amount which is sufficient to increase voltage gated calcium influx by at least 10%, preferably at least 20%, more preferably 30% and still more preferably 40%. Such alterations in voltage gated calcium influx can be measured by the assays described herein.

According to the invention α_{1I} isoform inhibitors also include “dominant negative” polypeptides derived for example from SEQ ID NOS:19 or 21. A dominant negative polypeptide refers to an inactive variant of a polypeptide, which, by interacting with the cellular machinery, displaces an active polypeptide from its interaction with the cellular machinery or competes with the active polypeptide, thereby reducing the effect of the active polypeptide. For example, a dominant negative receptor which binds a ligand but does not transmit a signal in response to binding of the ligand can reduce the biological effect of expression of the ligand. Likewise, a dominant negative human T-type calcium channel a α_{1I-1} subunit of an active complex (e.g. T-type calcium channel) can interact with the complex but prevent the activity of the complex (e.g. voltage gated calcium influx).

The end result of the expression of a dominant negative α_{1I-1} subunit polypeptide of the invention in a cell is a reduction in voltage gated calcium influx. One of ordinary skill in the art can assess the potential for a dominant negative α_{1I-1} subunit polypeptide, and using standard mutagenesis techniques to create one or more dominant negative “altered” polypeptides. For example, one of ordinary skill in the art can modify the sequence of the human T-type calcium channel α_{1I-1} subunit polypeptide (SEQ ID NO:19) by site-specific mutagenesis, scanning mutagenesis, partial gene deletion or truncation, and the like. See, Sambrook et al., Molecular Cloning: A Laboratory Manual, Second Edition, Cold Spring Harbor Laboratory Press, 1989. The skilled artisan then can test the population of mutagenized polypeptides for diminution in α_{1I-1} subunit activity (e.g., voltage gated calcium influx) and/or for retention of such an activity. Other similar methods for creating and testing dominant negative altered polypeptides of an isoform of the invention will be apparent to one of ordinary skill in the art.

Methods for reducing or inhibiting α_{1I} isoform activity, e.g. α_{1I-1} subunit activity in a cell are also encompassed by the present invention. The method proposes contacting the mammalian cell with an amount of an α_{1I-1} subunit inhibitor effective to inhibit voltage gated calcium influx in the mammalian cell. Such methods are useful in vitro for altering voltage gated calcium influx for the purpose of, for example, elucidating the mechanisms involved in stroke, pain, e.g., neuropathic pain, and traumatic brain injury and for restoring the voltage gated calcium influx in a cell having a defective α_{1I-1} subunit. In vivo, such methods are useful, for example, for reducing T-type voltage gated calcium influx, e.g., to treat stroke, pain, e.g., neuropathic pain, traumatic brain injury, or any condition in which the herein disclosed human T-type calcium channel α_{1I-1} subunit activity is elevated.

The invention also encompasses a method for increasing expression of a human T-type calcium channel α_{1I} isoform e.g., α_{1I-1} subunit in a cell or subject, especially is those subjects having a disorder characterized by a deficiency in voltage gated calcium influx. The method proposes contacting the cell with, or administering to the subject, an effective amount of α_{1I-1} subunit encoding nucleic acid or the polypeptide encoded thereby sufficient to increase voltage gated calcium influx in the cell or the subject. An increase in α_{1I-1} subunit activity, for example, can be measured by the assays described herein, e.g., assays of calcium influx. These assays are conducted according to standard procedures of testing calcium channel function (e.g. patch clamping, fluorescent Ca2+ influx assays) which require no more than routine experimentation. Using such methods, modulators of the activity of the α_{1I} isoforms of the invention effective at a particular voltages (e.g. complete membrane depolarization) can also be identified. Such compounds, in turn, will find use in selectively modulating calcium channel activity in conditions which may display voltage dependence. For example, following a stroke membranes are generally depolarized and such compounds may be active in selectively blocking calcium channel activity for treatment of stroke. Other uses will be apparent to one of ordinary skill in the art.

The invention also contemplates ex vivo gene therapy, a preferred procedure for which is detailed in U.S. Pat. No. 5,399,346 and references cited therein. A representative example of such therapy proposes introducing in vitro a functional copy of a gene, e.g., SEQ ID NOS:18 or 20, into a cell(s) of a subject in need of such therapy, e.g., subject containing a defective copy of the gene, and returning the genetically engineered cell(s) to the subject. It is noted that the functional copy of the gene is under operable control of regulatory elements that favor expression of the gene in the genetically engineered cell(s). In vivo gene therapy is also encompassed using appropriate vectors such as adenovirus, retroviruses, herpes virus, and targeted liposomes.

It is recognized that the preparations of the invention should be administered in effective amounts. An effective amount defines that amount of a pharmaceutical preparation that alone, or together with further doses, produces the desired response. In the case of treating a condition characterized by aberrant voltage gated calcium influx, the desired response is reducing or increasing calcium influx to a level which is within a normal range. Preferably, the change in calcium influx produces a detectable reduction in a physiological function related to the condition, e.g., a reduction in neurotoxicity following an epileptic attack or stroke. The responses can be monitored by routine methods. In the case of a condition where an increase in voltage gated calcium influx is desired, an effective amount defines that amount necessary to increase said influx in the target tissue. The converse is the case when a reduction in influx is desired. An increase or decrease in neurotransmitter release also could be measured to monitor the response.

The amount to be delivered will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons. In the event that a response in a subject is insufficient at the initial doses applied, higher doses (or effectively higher doses by a different, more localized delivery route) may be employed to the extent that patient tolerance permits. It is also recognized that lower doses will result from other forms of administration, such as intravenous administration. As well, multiple doses are also contemplated to achieve appropriate systemic levels of compound, although fewer doses typically will be given when compounds are prepared as slow release or sustained release medications.

When administered, the pharmaceutical preparations of the invention are preferable applied in pharmaceutically-acceptable amounts and in pharmaceutically-acceptably compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Inhibitors of the α_{1I} isoforms detailed herein, may be combined, optionally, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy. The pharmaceutical compositions may contain suitable buffering agents, including: acetic acid in a salt; citric acid in a salt; and phosphoric acid in a salt. As well, the pharmaceutical compositions may contain, suitable preservatives, such as: benzalkonium chloride; chlorobutanol; parabens and thimerosal.

The invention contemplates a variety of administration routes. The particular mode selected will depend, of course, upon the particular compound selected, the severity of the condition being treated and the dosage required for therapeutic efficacy. Suffice it to say that the methods of the invention may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intrathecal, intramuscular, or infusion. Intravenous or intramuscular routes are not particularly suitable for long-term therapy and prophylaxis. In addition, the pharmaceutical compositions of the invention may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration generally comprise a sterile aqueous preparation of one of the human T-type calcium channel α_{1I-1} subunit inhibitor or the human T-type calcium channel α_{1I-1} subunit nucleic acid molecules and polypeptides, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intrathecal, intramuscular, etc. administrations can be found in Remington's Pharmaceutical Sciences, Mack Publishing Co., Easton, Pa.

Other delivery systems can include time-release, delayed release or sustained release delivery systems such as the biological/chemical vectors is discussed above. Such systems can avoid repeated administrations of the active compound, increasing convenience to the subject and the physician. Many types of release delivery systems are available and known to those of ordinary skill in the art. Use of a long-term sustained release implant may be desirable. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

Another aspect of the invention features use of the novel polynucleotides, encoded α_{1I} proteins, encoded isoforms, inhibitors thereof including antibodies in a variety of diagnostic methods kits. Typically the kit will have a compartment containing either a defined α_{1I} subunit isoform of the invention, e.g., an α_{1I-1} polypeptide, polynucleotide, or a reagent which recognizes one or the other, e.g., antigen fragments or antibodies. Additionally the kit will include the reagents needed to carry out the assay in a separate compartment as well as instructions for use and proper disposal.

Use of an agent identified as described herein in an appropriate animal model is also within the scope of this invention. Thus, for example, an agent identified as described herein (e.g., the polynucleotides and encoded proteins of the invention, an α_{1I-1 or -2} modulating agent, an antisense α_{1I-1} or ₋₂ nucleic acid molecule, an α_{1I-1 or -2}-specific antibody, or an α_{1I-1 or -2} binding partner) can be used in an animal model to determine the efficacy, toxicity, or side effects of treatment with such an agent.

In accordance with the above, an aspect of the invention embraces using the novel polynucleotides of the invention to prepare a non-human transgenic animal. A “transgenic animal” is an animal having cells that contain DNA which has been artificially inserted into a cell, which DNA becomes part of the genome of the animal that develops from that cell. Preferred transgenic animals are primates, mice, rats, cows, pigs, horses, goats, sheep, dogs and cats. Animals suitable for transgenic experiments can be obtained from standard commercial sources such as Charles River (Wilmington, Mass.), Taconic (Germantown, N.Y.), Harlan Sprague Dawley (Indianapolis, Ind.), etc. Transgenic animals having a particular property associated with a particular disease can be used to study the affects of a variety of drugs and treatment methods on the disease, and thus serve as effective genetic models for the study of a number of human diseases. The α_{1I-1} subunit knockout and transgenic animals, for example, may be used as models for the study of disorders involving voltage gated calcium influx or dysfunctional calcium channels. A variety of methods are available for the production of transgenic animals associated with this invention. See e.g., Brinster et al., Proc. Nat. Acad. Sci. USA, 82: 4438 (1985); Brinster et al., Cell 27: 223 (1981); Costantini et al., Nature 294: 982 (1981); Harpers et al., Nature 293: 540 (1981); Wagner et al., Proc. Nat. Acad. Sci. USA 78:5016 (1981); Gordon et al., Proc. Nat. Acad. Sci. USA 73: 1260 (1976).

All patents, patent applications, and publications mentioned herein, whether supra or infra, are each incorporated by reference in its entirety. The broad scope of this invention is best understood with reference to the following examples, which are not intended to limit the invention to the specific embodiments described below.

### EXAMPLES

Some of the standard methods are described or referenced, e.g., in Maniatis et al. (1982) Molecular Cloning, A Laboratory Manual, Cold Spring Harbor Laboratory, Cold Spring Harbor Press; Sambrook et al. (1989) Molecular Cloning: A Laboratory Manual, (2d ed.), vols. 1-3, CSH Press, N.Y.; or Ausubel et al. (1987 and Supplements) Current Protocols in Molecular Biology, Greene/Wiley, New York; Innis et al. (eds.) (1990) PCR Protocols: A Guide to Methods and Applications Academic Press, N.Y. Methods for protein purification include such methods as ammonium sulfate precipitation, column chromatography, electrophoresis, centrifugation, crystallization, and others. See, e.g., Ausubel et al. (1987 and periodic supplements); Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering Principle and Methods 12:87-98, Plenum Press, N.Y.; and Crowe et al. (1992) OIAexpress: The High Level Expression & Protein Purification System QUIAGEN, Inc., Chatsworth, Calif.

### Example 1

### Isolation of DNA Encoding the Human Calcium Channel α_{1I} Subunit

### A. RNA Isolation

Human medullary thyroid carcinoma cells (TT cells; ATCC Accession No. CRL1803) were grown in DMEM medium supplemented with 10% fetal calf serum at 37° C. in 5% CO 2 atmosphere and total cytoplasmic RNA was isolated from forty 10 cm plates using a “midi-prep” RNA isolation kit (Qiagen) as per the manufacturer's instructions. The protocol entails the use of the detergent NP40 which lyses the cell membrane under mild conditions such that the nuclear membrane remains intact thereby eliminating incompletely spliced RNA transcripts from the preparation.

PolyA+ RNA was isolated from total cytoplasmic RNA using two passes over an oligo(dT)-cellulose column. Briefly, 2-3 mg of total cytoplasmic RNA was resuspended in NETS buffer (500 mM NaCl 10 mM EDTA, 10 mM Tris, pH 7.4, 0.2% SDS) and passed slowly over a column containing 0.5 g of oligo(dT)-cellulose (Collaborative Research) equilibrated in NETS buffer. The column was washed with 30 mils of NETS buffer and polyA+ RNA was eluted using 3 mls of ETS buffer (10 mM EDTA, 10 mM Tris, pH 7.4, 0.2% SDS). The ionic strength of the polyA+ RNA-containing buffer was adjusted to 500 mM NaCl and passed over a second oligo(dT)-cellulose column essentially as described above. Following elution from the second column, the polyA+ RNA was precipitated twice in ethanol and resuspended in H₂O.

### B. Library Construction

Double stranded cDNA (dscDNA) was synthesized according to standard methods [Gubler et al. (1985) Gene 25:263-269; Lapeyre et al. (1985) Gene 37:215-220]. Briefly, first strand cDNA synthesis was initiated using TT cell polyA+ RNA as a template and using random primers and Moloney Murine Leukemia Virus reverse transcriptase (MMLV-RT). The second strand was synthesized using a combination of E. coli DNA polymerase, E. coli DNA ligase and RNase H. Regions of single stranded DNA were converted to double-stranded DNA using T4 DNA polymerase generating blunt-ended double stranded fragments. EcoRI restriction endonuclease site adapters:

| | |
|---|---|
| 5′ CGTGCACGTCACGCTAG 3′ | (SEQ ID NO:1) |
| 3′ GCACGTGCAGTGCGATCTTAA 5′ | (SEQ ID NO.2) |

were ligated to the double-stranded cDNA using a standard protocol [see, e.g., Sambrook et al. (1989) IN: Molecular Cloning, A Laboratory Manual, Cold Spring Harbor Laboratory Press, Chapter 8]. The double-stranded cDNA with the EcoRI adapters ligated was purified away from the free or unligated adapters by column chromatography using Sepharose CL-4B resin followed by size selection of the cDNA on a 1.2% agarose gel. After visualizing the resolved DNA using ethidium bromide, two fractions of cDNA, >3.5 kb and 1.0-3.5 kb, were isolated from the gel and inserted into the vector λgt10.

The ligated λgt10 containing the cDNA insert was packaged into λ phage virions in vitro using the Gigapack II Gold packaging (Stratagene, La Jolla, Calif.) kit. Using this method, phage libraries of ˜1.5×10⁶ recombinants for the cDNA >3.5 kb fraction and ˜10×10⁶ recombinants for the DNA fraction between 1.0 and 3.5 kB were obtained.

C. Isolation of DNA Encoding a Human α_{1I} Calcium Channel Subunit and Construction of DNA Encoding a Full-Length α_{1I} Subunit

1. Reference list of Partial Human α_{1I} DNAs

The full-length α_{1I} cDNA sequence is set forth in SEQ ID NO:18. A list of partial cDNA clones and PCR fragments used to characterize the α_{1I} sequence and the nucleotide position of each DNA relative to the full-length α_{1I} cDNA sequence is shown below. The isolation and characterization of these clones are described below.

PCR1009/1010 nt 715 to 940 of SEQ ID NO:18

clone 1.401 nt 1 to 3799 of SEQ ID NO:18

clone 1.404 nt 160 to 3910 of SEQ ID NO:18

PCR136/140 nt 3021 to 4225 of SEQ ID NO:18

PCR141/142 nt 3953 to 5337 of SEQ ID NO:18

PCR143/151 nt 4952 to 6745 of SEQ ID NO:18

2. Isolation and Characterization of Individual Clones

DNA encoding a small region of the human α_{1I} subunit encoded in TT cells was isolated by PCR. Two oligonucleotides were synthesized based on expressed sequence tags (ESTs) in the Genbank database with homology to the human α_{1H} subunit. A 5′ primer (SEQ ID NO:3) based on EST H55223 and a 3′ primer (SEQ ID NO:4) based on EST H55617 were used in amplification reactions with TT cDNA and PfuTurbo DNA polymerase (Stratagene). Reactions were performed at 95° C. for 5 min, followed by 30 cycles of 20 sec@95° C., 20 sec@60° C. and 3 min@75° C. Amplified DNA products were subjected to electrophoresis on an agarose gel and gel purified using standard methods. The sequence of the 226 bp DNA fragment, designated PCR1009/1010 (SEQ ID NO:12), was determined and found to share 76.5% sequence identity to the human α_{1H} subunit (Genbank Accession #AF073931). PCR1009/1010 was used as a DNA probe to isolate DNA encoding a human α_{1I} calcium channel subunit.

Approximately 1.5×10⁶ recombinants of the TT cell phage library containing inserts >3.5 kb were plated and lifts prepared from each plate. The lifts were probed with radiolabelled PCR1009/1010 using standard hybridization conditions, the filters were washed under high stringency and 4 positive plaques were identified. Three positives, 1.401, 1.402 and 1.404, were chosen for plaque purification and additional characterization. Restriction endonuclease digestion of purified DNA isolated from clones 1.401, 1.402 and 1.404 with EcoRI indicated that these clones contain inserts of approximately 3700 nt, 2500 nt and 3700 nt, respectively. DNA sequencing of the inserts from clones 1.401 (SEQ ID NO:13) and 1.404 (SEQ ID NO:14) indicated that the clones contain overlapping sequences that encode an open reading frame of approximately 3.5 kb that encodes approximately 60% of the α_{1I} subunit.

DNA encoding the 3′-end of the human α_{1I} calcium channel subunit was isolated by PCR.

Briefly, the 3′ end of the α_{1I} sequence was divided into 3 sections and each section amplified separately.

An ˜1.2 kb DNA fragment, designated PCR136/140 (SEQ ID NO:15) was amplified from TT cell cDNA with a 5′ primer (SEQ ID NO:5) based on sequence near the 3′ end of clone 1.404 and a 3′ primer (SEQ ID NO:6) based on Genbank entry HS172B20. Amplification reactions were set as follows: 1 cycle, 1 min@96° C.; 10 cycles, 30 sec@96° C./45 sec@65° C. (−0.5° C./cycle)/2 min@74° C.; 20 cycles, 30 sec@96° C./45 sec@60° C./2 min@74° C.; and 1 cycle, 7 min@74° C.

An ˜1.4 kb DNA fragment, designated PCR141/142 (SEQ ID NO:16), was amplified from TT cell cDNA with a 5′ and 3′ primers (SEQ ID NO:7 and SEQ ID NO:8, respectively) based on Genbank entry HS172B20. Amplification reactions were set as follows: 1 cycle, 1 min@96° C.; 30 cycles, 30 sec@96° C./45 sec@ 59° C./2 min@74° C.; and 1 cycle, 7 min@74° C.

An ˜1.8 kb DNA fragment, designated PCR143/151 (SEQ ID NO:17) was amplified from TT cell cDNA using a nested PCR strategy. Initially, 5′ and 3′ primers (SEQ ID NO:9 and SEQ ID NO:10, respectively) based on Genbank entry HS172B20 were used in an amplification reaction under the following conditions: 1 cycle, 1 min@96° C.; 30 cycles, 30 sec@96° C./3 min@68° C.; and 1 cycle, 7 min@74° C. Ten percent of the amplification reaction was used as the template in a second reaction with the same 5′ primer (SEQ ID NO:9) and a new 3′ primer (SEQ ID NO:11). The reactions were set as: 1 cycle, 1 min@96° C.; 30 cycles, 30 sec@96° C./45 sec@62° C./3 min@74° C.; and 1 cycle, 7 min@74° C.

Amplified DNA products were subjected to electrophoresis on agarose gels and gel purified using standard methods. The sequence of the PCR 136/140, PCR141/142 and PCR143/151 fragments was determined and found to share 59.4%, 81.9% and 56.5% sequence identity, respectively, to the human α_{1H} subunit.

3. Construction of a Full-Length α_{1I}-Encoding DNA Clone

Portions of these partial cDNA clones can be ligated to generate a full-length α_{1I} cDNA using common restriction endonuclease sites shared amongst the α_{1I}-encoding fragments. For example, the construction of a full-length α_{1I} cDNA can be prepared in two steps as described in detail below: 1) DNA encoding the 5′-end of α_{1I} present in clone 1.404 can be combined with PCR136/140 and PCR141/142 using a common AvrII restriction endonuclease site (nt 3076 to 3081 of SEQ ID NO:18) and a common BglII site (nt 4104 to 4109 of SEQ ID NO:18); and 2) the DNA of step 1) encoding the amino terminus of α_{1I} can be combined with the carboxyl terminal sequences of α_{1I} encoded in PCR143/151 using the common HindIII site (nt 5148 to 5153 of SEQ ID NO:18) shared between clone PCR141/142 and PCR143/151. The resulting full-length human α_{1I} calcium channel cDNA (SEQ ID NO:18) contains a coding sequence of 5943 nts and encodes an α_{1I} protein that is 1981 amino acid residues in length (SEQ ID NO:19).

4. Alternative Splicing

Clone 1.401 contains a 54 nt insert relative to clone 1.404 and is contained within SEQ ID NO:20. The insertion results in an α_{1I} subunit containing an additional 18 amino acids (SEQ ID NO:21). The location of the insert is depicted in the alignments shown above.

### Example 2

### Generation of a Stable Cell Line for the Human Calcium Channel α_{1I} Subunit

### A. Construction of Expression Vector for α_{1I} Subunit

In order to increase the expression level of α_{1I} calcium channel the Kozak sequence was inserted before translation start codon. The primers for PCR reaction were 5′-CTT AAG CC ACC ATG GCT GAG AGC GCC TCC CCT CCT CA (SEQ ID NO: 22) and 3′-TAG AGC ACT GGT CTG TGG GCA AGG CGG CCG C (SEQ ID NO:23). The PCR reactions were set as: 1 cycle, 2 min@95° C., 30 cycle, 30 sec@95° C./30 sec@68° C./6 min@72° C./; and 1 cycle 10 min@72° C. Amplified PCR product was subjected to electrophoresis on 1% agarose gels and gel purified using standard methods. Then the purified PCR product was cloned into Zero Blunt TOPO vector by using Zero Blunt TOPO PCR cloning kit (Invitrogen, CA). The expression vector (pcDNA4/TO, Invitrogen, CA) used in this study contained the tetracycline-inducible CMV promoter (PCMV) and was constructed by ligating the 6-kilobase Afl II/Not I fragment containing the α_{1I} cDNA into the Afl II/Not I-digested pcDNA4/TO vector.

### B. Transfection of α_{1I} Subunit in a Mammalian Cell Line

The Tetracycline-Regulated Expression cell lines (T-REx™-293) stably expressed the tetracycline repressor were purchased from Invitrogen and maintained in DMEM medium with 10% fetal bovine serum, 2 mM L-glutamine, 50 U/ml penicillin and 50 μg/ml streptomycin, and 5 μg/ml of blasticidin. To establish stably transfected cell lines, 2.5 μg of α_{1I}-pcDNA4/TO DNA was incubated with FuGENE 6 transfection reagent (15 μl in 400 μl medium, Roche, Indianapolis, Ind.) for 30 min, and then the complex mixture from above preparation was added into T-Rex™-293 cells. Two days after transfection, cells were selected in presence of 200 μg/ml of zeocin under limiting dilution. Individual colonies were isolated using sterile cloning cylinders and expanded, and cloned cells were screened for α_{1I} expression initially by calcium influx assay and later by electrophysiological recordings after the addition of 0.1 μM tetracycline to the growth medium. An α_{1I} cell line (B21) was selected from a range of stable transfects.

### Example 3

### Production of Antisera and Immunoblot Analyses

Rabbit polyclonal antibody was generated against peptides corresponding to specific sequence of human α_{1I} subunit in New Zealand white rabbits. The peptide was commercially synthesized and purified (Cambridge research biochemicals, UK). Antigenic epitopes comprised the amino acid sequence 1067-1088 (KDVFTKMGDRGDRGEDEEEID). The sequence has 90% homology to the rat sequence (Genbank Accession #AF086827). A rabbit was injected with purified KLH conjugated-peptide in complete Freund's adjuvant, and received five subsequent booster of the same antigen in incomplete Freund's adjuvant. The obtained antiserum #732 was titrated with an enzyme-linked immunosorbent assay (ELISA) against the purified peptide and was shown to specifically recognize human alpha 11 at a dilution of 1:2000. For Western blot analysis, the wild type T-Rex, non-induced B21 and induced B21 cells were washed once with PBS and homogenized using a homogenizer in 20 mM Tris buffer. The homogenates were centrifuged at 1000 RPM for 10 min at 4° C. The supernatant was collected and then centrifuged at 18,000 RPM for 45 min at 4° C. The pellet was resuspended as 1-2 ug/ul concentration of membrane protein. The membrane proteins (20 ug/lane) from three different cells were electrophoresed in 1-mm-thick NuPAGE-3-8% Tris-Acetate gel (Invitrogen, CA) at 150 V for 1.5 hr at room temperature. Proteins resolved were transferred by electroblotting to PVDF membranes (Invitrogen, CA) at 30 V for 1.5 hr. The membranes were blocked for 1 h in 5% milk in Tris-buffered saline containing 0.1% Tween (TBST). The membranes were incubated with rabbit anti-α1 antisera #732 at dilution of 1:500 in 5% milk/TBST for 60 min, washed three times in TBST, and incubated with a horseradish peroxidase-conjugated donkey anti-rabbit IgG at a 1:5000 in TBST for 60 min. The membrane were washed three times in TBST and subjected to enhanced chemiluminescence (ECL-plus) detection according to the manufacturer's instructions (Amersham, N.J.).

### Example 4

### Functional Expression of the Human Calcium Channel α_{1I} Subunit

### A. Calcium Influx Measured by FLIPR

Intracellular calcium concentration was monitored by the FLIPR™ technique (Molecular Devices, Sunnyvale, Calif.). Briefly, the cells were seeded into black 96 well plates with clear bottoms at a density of 35,000 cells/well and cultured for 5 hr. After 0-2 μM tetracycline was added into the 96-well plate for 18 hr, the cells were incubated with Fluo-3AM (final concentration 4 μM, Molecular probe) in calcium free Hank's buffer supplement with 0.1% BSA and 20 mM HEPES for 1 hr at 37° C., 5% CO₂ and 95% O₂. The cells were washed four times with assay buffer and the cell plate was placed into the FLIPR™ to monitor cell fluorescence (λ_{EX}=488 nm, λ_{EM}=540 nm) before and after the addition of antagonists and agonists.

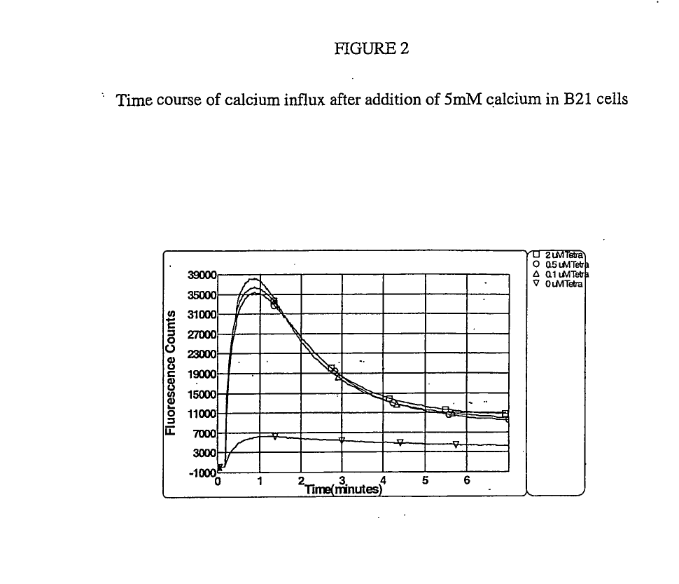

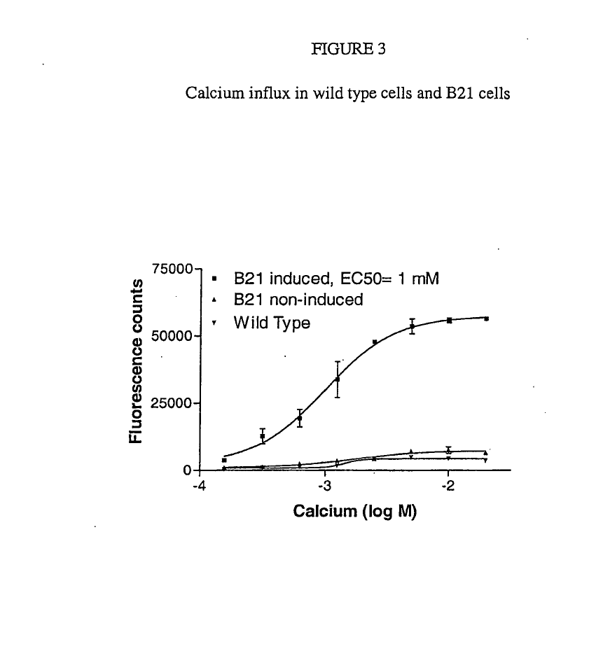

Results: FIG. 2 shows the time course of calcium influx through the α_{1I} channel in B21 cells induced by 0, 0.1, 0.5 and 2 μM tetracycline. This Ca²⁺ influx after Ca²⁺ addition was concentration-dependent in B21 cells induced by 0.1 μM tetracycline (FIG. 3). The EC50 was 1 mM in B21 cells. No or minimal increases in cytosolic Ca²⁺ were detected in the cells in the absent of tetracycline or in wild type cells.

To confirm this increase in Ca²⁺ influx through the T-type Ca²⁺ channel, several Ca²⁺ channel antagonists including mibefradil were used in this invention. Mibefradil and pimozide, selective for T-type calcium channel, completely inhibited Ca²⁺ influx, but not L-type calcium channel antagonists, nimodipine and verapamil. The potency of the inhibitory effect of mibefradil and other calcium channel antagonists on calcium influx through the α_{1I} channel was shown in table 1.

TABLE 1
IC₅₀ values of calcium channel antagonists for inhibition of calcium
influx in B21 cell induced by 0.1 uM tetracycline

| Antagonists | IC₅₀ (nM) | n |
|---|---|---|
| Mibefradil | 166 ± 51 | 3 |
| Pimozide | 1004 ± 109 | 3 |
| Nimodipine | 9511 ± 788 | 3 |
| Verapamil | 10632 ± 1974 | 3 |

Values are the mean ± S.E.

### B. Electrophysiological Characterization of B21 Cell Line

Whole-cell patch clamp recordings (V_{holding}=−100 mV) were performed on the B21 cell line using standard voltage clamp recording techniques. Briefly, recording from HEK293 cells expressing α_{1I} T-type channel was done with an Axon Instruments model 200B Integrating Patch Clamp amplifier or with an Axon Instruments 700A multi-clamp amplifier and data were digitized at 10 kHz by a Digidata 1320A analog-to-digital converter. Data were analyzed with Axon Instruments software, Clampex version 8.2, or later. For whole-cell recording, patch pipettes (2-5 MΩ) contained (in mM): 125 CsMeSO3, 10 HEPES, 8 NaCl, 1 CaCl2, 10 EGTA, 2 Mg-ATP, 0.3 GTP, or 135 CsCl, 10 HEPES, 5 EGTA, 3 Mg-ATP, 2 Na-ATP, 1 Na₂ GTP, 2 MgCl₂, finally, pH was adjusted to 7.2 with CsOH. Extracellular solution was based on Tyrode solution with minor alterations (in mM): 130 NaCl, 4 KCl, 30 glucose, 25 HEPES, 1 MgCl₂, 2 CaCl₂, pH adjusted to 7.4 with NaOH. Access resistance was monitored online and was typically <12 MΩ. Cells with excessive leak current were excluded from analysis.

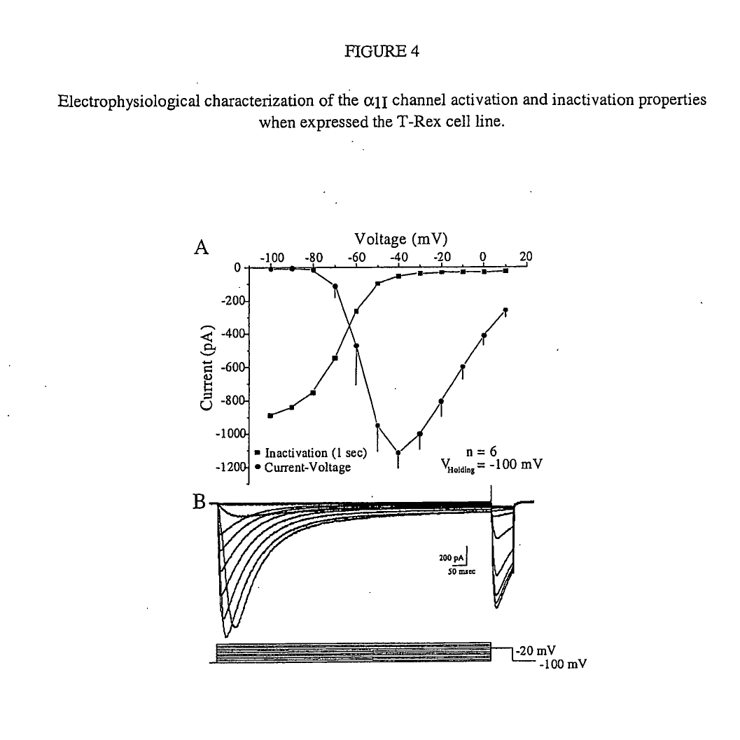

For establishing an α_{1I}-type calcium channel current-voltage relationship, the protocol shown in FIG. 4B was used. From a holding potential of −100 mV, depolarizing steps to various potentials were applied for 1 second, followed by a brief 40-msec step pulse to −20 mV. This protocol was used to demonstrate both the voltage dependence of activation and I-V relationship of the channel, as well as the extent of channel inactivation in 1 second at different membrane potentials. The mean peak current amplitudes from these experiments are displayed in FIG. 4. Plotting the peak current from the successive 1-second step-pulses results in an I-V relationship typical of low voltage activated T-type channels (FIG. 4A, filled circles). The channel activates at more negative potentials than other types of calcium channels. In FIG. 4 the activation threshold voltage was −70 mV, which is significantly more negative than in the high-voltage activated L- and N-type calcium channels. Notably, the maximum peak current occurred at −40 mV and was in excess of 1 nA, indicating significant expression of functional channels.

The α_{1I} T-type calcium channel current expressed in the B21 line also showed a voltage-dependent inactivation (FIG. 4A, filled squares). When the I-V plot and the inactivation plots are compared, as in FIG. 4 A, a small “window” current is seen that peaks at approximately −63 mV. This “window” current is a hallmark for many other T-type calcium channels. Therefore, all data indicate that the B21 cell line expresses a voltage dependent T-type calcium current.

To further characterize the T-type calcium channel current in the B21 cell line was a T-type calcium channel current, selective T-type calcium channel inhibitors were used to test whether the B21 cell line expressed channels that were sensitive to these T-type channel-selective inhibitors. For establishing an α_{1I} T-type calcium channel antagonist IC₅₀, a control baseline T-type calcium current was elicited by depolarizing the cell with a standard square pulse from holding potential of 100 mV, to a depolarized potential of −20 mV for 80 milliseconds, repeating the cycle every 10 seconds. Once stable current amplitudes were observed, an extracellular solution containing a known concentration of antagonist, either mibefradil or pimozide, was flowed into the bath surrounding the cell. Percent inhibition of the peak control calcium current was determined from the reduction in the peak of the evoked calcium current in the presence of the compound, and then compared to baseline peak current. The percent inhibition in the peak current was recorded as a function of antagonist concentration.

An estimated IC₅₀ of the compound is reported from the simple equation:

Estimated IC₅₀=([Compound concentration (M)×(100/percent inhibition)]−[compound concentration (M)])×1,000,000,000

For example: Compound A has 20 percent inhibition of peak current at 10 mM.
Therefore, using the above equation:

(((0.00000001*(100/20))−0.00000001)*1000000000)=40 nM

Thus, compound A would have an estimated IC₅₀ of 40 nM.

This equation is applied only when the percent inhibition fell between 20% and 80% inhibition (linear range). The estimated IC₅₀ derived from the concentration of compound giving a percent inhibition closest to 50% is reported when multiple values are measured at differing concentrations.

Based on the Electrophysiological Characterization of the B21 cell line, it was found that mibefradil had an estimated IC₅₀ of 427 nM and Pimozide had an estimated IC₅₀ of 216 nM (Table 2). These results, in addition to studies on the current-voltage relationship and the voltage-dependent inactivation of the current, confirm that the B21 cell line expresses a channel whose current posses similar kinetics, activation profile, inactivation profile, and antagonist sensitivity as other published reports of T-type calcium channels. However, the B21 line expresses a maximum current magnitude (>1 nA) that significantly exceeds any other reported cell type or cell line expressing the α_{1I} T-type calcium channel. Therefore, because this cell line expresses the unique α_{1I} T-type calcium channel current, but has a much larger and more easily measurable current, it is thus believed to be a far superior tool with which enable one skilled in the art to screen and test T-type channel modulators.

TABLE 2
Estimated IC₅₀ values for published T-type calcium channel antagonists in
voltage-clamp experiments at −100 mV holding potential

| Antagonists (conc.) | Est IC₅₀ (nM) | n | % Inhibition (Ave ± SEM) |
|---|---|---|---|
| Mibefradil (500 nM) | 427 | 3 | 54 ± 4 |
| Pimozide (100 nM) | 216 | 4 | 32 ± 2 |

Summary of sequences depicting full length cDNA and protein sequences corresponding to each of the novel α_{1I} isoforms of the invention disclosed herein.

SEQ ID NO:18 depicts the nucleic acid sequence of a variant of the invention (Variant #1 e.g., α_{1I-1} subunit).

SEQ ID NO:19 depicts the deduced amino acid sequence of α_{1I-1} subunit.

SEQ ID NO:20 depicts the nucleic acid sequence of a variant of the invention e.g., α_{1I-2} subunit.

SEQ ID NO:21 depicts the deduced amino acid sequence encoded by SEQ ID NO:20.

1. An isolated nucleic acid molecule of an alternative splicing variant of a human T-type calcium channel subunit, comprising a sequence of nucleotides encoding a T-type calcium channel α_{1I-1} subunit selected from the group consisting of:
(a) a sequence of nucleotides that encodes a human T-type calcium channel α_{1I-1} subunit and comprises the sequence of nucleotides set forth in one of SEQ ID NO.: 18;
(b) a sequence of nucleotides having at least 95% sequence identity or is exactly complementary to the nucleotide sequence set forth in SEQ ID NO.: 18,
(c) a nucleotide sequence varying from the nucleotide sequence specified in (a) or (b) as a result of degeneracy of the genetic code;
(d) biologically active fragments of (a), (b), or (c), that encodes polypeptide capable of forming a functional T-type calcium channel.

2. (canceled)

3. A substantially pure polypeptide comprising an amino acid sequence encoded by the nucleotide sequence as set forth in one of SEQ ID NOS.:18 or 20.

4. A substantially pure polypeptide comprising an amino acid sequence as set forth in one of SEQ ID NOS.:19 or 21.

5. A substantially pure polypeptide which has at least 80% identity to the amino acid sequence of SEQ ID NO.: 19, which may include up to Na amino acid alterations over the entire length of SEQ ID NO.: 19, wherein Na is the maximum number of amino acid alterations, and is calculated by the formula
Nₐ=Xₐ−(Xₐ Y),
in which Xa is the total number of amino acids in SEQ ID NO.: 19, and Y has a value of 0.80, wherein any non-integer product of Xₐ and Y is rounded down to the nearest integer prior to subtracting such product from Xₐ.

6. (canceled)

7. An expression vector comprising the nucleic acid molecule of claim 1 operably linked to a regulatory nucleotide sequence that controls expression of the nucleic acid molecule in a suitable host cell.

8. A recombinant host cell transfected by the expression vector of claim 7.

9. The cell of claim 8 which is also transformed with DNA expression vectors encoding additional calcium channel subunits necessary and sufficient for assembly of a functional voltage-gated calcium channel.

10. (canceled)

11. (canceled)

12. (canceled)

13. (canceled)

14. (canceled)

15. An isolated nucleic acid molecule of an alternative splicing variant of a human T-type calcium channel subunit, comprising a sequence of nucleotides encoding a T-type calcium channel α_{1I-2} subunit selected from the group consisting of:
(a) a sequence of nucleotides that encodes a human T-type calcium channel α_{1I-1} subunit and comprises the sequence of nucleotides set forth in one of SEQ ID NO.:20;
(b) a sequence of nucleotides having at least 95% sequence identity or is exactly complementary to the nucleotide sequence set forth in SEQ ID NO.:20,
(c) a nucleotide sequence varying from the nucleotide sequence specified in (a) or (b) as a result of degeneracy of the genetic code;
(d) biologically active fragments of (a), (b), or (c), that encodes polypeptide capable of forming a functional T-type calcium channel.

16. A method for identifying candidate compounds capable of binding to the polypeptide of claim 3 and modulating its activity the method comprising: (i) contacting a candidate compound with the substantially pure polypeptide of claim 3; and (ii) determining the effect of said candidate compound on the biological activity of said protein or polypeptide and selecting those compounds which show a significant effect on said biological activity.

17. A method according to claim 16, wherein the compound is an agonist and the measured effect is increase in the biological activity.

18. A method according to claim 17, wherein the compound is an antagonist and the effect is decrease in the biological activity.

19. A method for detecting an α_{1I} isoform in a first biological sample, comprising the steps of: (a) contacting a detectable probe with said biological sample suspected of containing said variant under conditions favoring the formation of a complex between said probe and any said variant; and (b) detecting said complex wherein the presence of said complex correlates with the presence of the desired amino acid in said biological sample.

20. The method according to claim 19, wherein said probe is an antibody.

21. The method according to claim 19, wherein said probe is an immunologically active polypeptide specific for said isoform.

22. (canceled)

23. (canceled)

24. (canceled)

25. (canceled)

26. (canceled)

27. (canceled)

28. (canceled)

29. (canceled)

30. (canceled)

31. A method for treating a subject having a stroke, epileptic seizure, or traumatic brain injury comprising administering to a subject in need of such treatment an inhibitor of the human T-type calcium channel α_{1I-1} subunit polypeptide in an amount effective to inhibit voltage regulated calcium influx.

32. The method of claim 31, wherein the inhibitor is selected from the group consisting of an antibody which selectively binds the human T-type calcium channel α_{1I-1} subunit polypeptide, an antisense nucleic acid which binds a nucleic acid encoding human T-type calcium channel α_{1I-1} or an α_{1I-2} subunit polypeptide and a dominant negative human T-type calcium channel α_{1I-1} or an α_{1I-2} subunit polypeptide.

33. (canceled)

34. (canceled)

35. (canceled)

36. A method for identifying lead compounds for a pharmacological agent useful in the treatment of disease associated with increased or decreased voltage regulated calcium influx mediated by a human T-type calcium channel comprising:
(i) providing a cell expressing a human T-type calcium channel isoform subunit polypeptide designated herein as α_{1I-1} or α_{1I-2};
(ii) contacting the cell with a candidate pharmacological agent under conditions which, in the absence of the candidate pharmacological agent, to thereby cause a first amount of voltage regulated calcium influx into the cell; and
(iii) determining a test amount of voltage regulated calcium influx as a measure of the effect of the lead compounds for a pharmacological agent on the voltage regulated calcium influx mediated by a human T-type calcium channel, wherein (a) the test amount of voltage regulated calcium influx which is less than the first amount indicates that the candidate pharmacological agent is a lead compound for a pharmacological agent which reduces voltage regulated calcium influx and (b) wherein a test amount of voltage regulated calcium influx which is greater than the first amount indicates that the candidate pharmacological agent is a lead compound for a pharmacological agent which increases voltage regulated calcium influx.

37. The method of claim 36, further comprising loading said cell with a calcium-sensitive dye which is detectable in the presence of calcium, wherein the calcium-sensitive dye is detected as a measure of the voltage regulated calcium influx.

38. A method for identifying compounds which selectively bind a human T-type calcium channel α_{1I-1} subunit isoform comprising, (i) providing a test cell preparation, wherein said cell expresses a human T-type calcium channel α_{1I-1} subunit isoform, (ii) providing a control cell preparation, wherein said cell expresses a human T-type calcium channel non-α_{1I-1} subunit isoform, with the proviso that the cell in the cell preparation is identical to the test cell except for the expression of a non-α_{1I-1} isoform being expressed, (iii) contacting the test cell preparation and the control cell preparation with a compound, and (iv) determining the binding of the compound to the test cell preparation and the control cell preparation, wherein a compound which binds the test cell preparation but does not bind the control cell preparation is a compound which selectively binds the human T-type calcium channel α_{1I-1} subunit isoform.

39. (canceled)

40. (canceled)

41. (canceled)

42. (canceled)

43. An isolated antibody which specifically binds to a polypeptide comprising an amino acid sequence selected from the group consisting of:
(a) an amino acid sequence of SEQ ID NOS.: 19 or 21,
(b) a naturally-occurring amino acid sequence having at least 95% sequence identity to the sequence of SEQ ID NOS.: 18 or 20, wherein said naturally-occurring amino acid sequence has the ability to regulate voltage gated calcium influx under physiological conditions and
(c) an immunogenic fragment derived from one of SEQ ID NO.:19 or 21.

44. (canceled)

45. A recombinant human cell line which has been engineered to express a heterologous protein, the cell line comprising at least one host cell transformed or transfected with a heterologous nucleic acid molecule of claim 1 that expressed an α_{1I} isoform polypeptide.

46. (canceled)

47. A method of producing the recombinant protein according to claim 3, comprising:
(a) inserting the nucleic acid sequence as set forth in one of SEQ ID NO.:19 or 21 or a fragment or variant thereof into an expression vector;
(b) transferring the expression vector into a host cell; or transfecting or transforming a host cell with the expression vector of step (a) above;
(c) culturing the host organism under conditions appropriate for amplification of the vector and expression of the protein; and
(d) harvesting the recombinant protein from the culture.

48. A method for identifying compounds that modulate the activity of T-type calcium channel α_{1I-1} subunit, the method comprising:
comparing the difference in the amount of transcription of a reporter gene in a cell in the presence of the compound with the amount of transcription in the absence of the compound, or with the amount of transcription in the absence of a heterologous T-type calcium channel α_{1I-1} subunit, whereby compounds that modulate the activity of the heterologous calcium channel subunit in the cell are identified, wherein the cell comprises a nucleic acid molecule that encodes a reporter gene construct containing a reporter gene in operative linkage with one or more transcription control elements that is regulated by a calcium channel and furthermore the cell is a eukaryotic cell transfected with a nucleic acid molecule comprising the coding portion of the sequence of nucleotides set forth in one of SEQ ID NO.: 18 or 20.

49. A method for identifying a test compound capable of modulating the activity of T-type calcium channel α_{1I-1} subunit, the method comprising:
(i) suspending a eukaryotic cell in a solution containing the compound and a calcium channel selective ion;
(ii) depolarizing the cell membrane of the cell, and
(iii) detecting the current or ions flowing into the cell,
wherein the eukaryotic cell comprises a functional calcium channel that contains at least one subunit encoded by a heterologous nucleic acid comprising the coding portion of the sequence of nucleotides set forth in SEQ ID NOS.: 18 or 20, and
wherein the current that is detected is different from that produced by depolarizing the same or a substantially identical cell in the presence of the same calcium channel selective ion but in the absence of the test compound.

50. The method of claim 49, wherein prior to the depolarization step the cell is maintained at a holding potential which substantially inactivates calcium channels that are endogenous to the cell.
